(19) United States
(12) Patent Application Publication
(10) Pub. No.: US 2022/0257581 A1
(43) Pub. Date: Aug. 18, 2022

(54) Treatment of Cancer

(71) Applicant: Sumitomo Dainippon Pharma Oncology, Inc., Cambridge, MA (US)

(72) Inventors: David J. Bearss, Alpine, UT (US); Stephen Patrick Anthony, Herber City, UT (US); Michael Vincent McCullar, Walnut Creek, CA (US)

(21) Appl. No.: 17/596,807

(22) PCT Filed: Jul. 8, 2020
(86) PCT No.: PCT/US2020/041187

§ 371 Date: Dec. 17, 2021

Related U.S. Application Data
(60) Provisional application No. 63/037,561, filed on Jun. 10, 2020.
(60) Provisional application No. 63/029,938, filed on May 26, 2020.
(60) Provisional application No. 62/871,540, filed on Jul. 8, 2019.

(51) Int. Cl. A61K 31/453; A61K 31/454; A61K 31/573; A61K 31/7068; A61K 31/704; A61K 31/136; A61P 35/02; A61P 35/00; A61K 31/706; A61K 31/675

(52) U.S. Cl. CPC A61K 31/453; A61K 31/454; A61K 31/573; A61K 31/7068; A61K 31/675; A61K 31/136; A61P 35/02; A61P 35/00; A61K 31/706; A61K 31/704

(57) ABSTRACT

Provided herein are methods of treating a hematologic cancer in a subject in need thereof, comprising administering to the subject an effective amount of alvocidib, or prodrug thereof, or a pharmaceutically acceptable salt of the foregoing. The subject is in complete remission from the hematologic cancer and measurable residual disease (MRD)-positive following administration of a prior therapy that does not include alvocidib, or a prodrug thereof, or a pharmaceutically acceptable salt of the foregoing, and is MRD-negative following administration of alvocidib, or a prodrug thereof, or a pharmaceutically acceptable salt of the foregoing. Other methods are also provided in accordance with other aspects of the invention.

### RELATED APPLICATIONS

This application claims the benefit of U.S. Provisional Application No. 63/037,561, filed on Jun. 10, 2020, U.S. Provisional Application No. 63/029,938, filed on May 26, 2020, and U.S. Provisional Application No. 62/871,540, filed on Jul. 8, 2019. The entire teachings of these applications are incorporated herein by reference.

### SUMMARY

Provided herein is a method of treating a hematologic cancer in a subject in need thereof, comprising administering to the subject an effective amount of alvocidib, or a prodrug thereof, or a pharmaceutically acceptable salt of the foregoing, wherein the subject is in complete remission from the hematologic cancer and measurable residual disease (MRD)-positive following administration of a prior therapy that does not include alvocidib, or a prodrug thereof, or a pharmaceutically acceptable salt of the foregoing, and is MRD-negative following administration of alvocidib, or a prodrug thereof, or a pharmaceutically acceptable salt of the foregoing.

Also provided herein is a method of treating a hematologic cancer in a subject in need thereof, comprising administering to the subject a maintenance therapy comprising an effective amount of alvocidib, or a prodrug thereof, or a pharmaceutically acceptable salt of the foregoing, wherein the subject is in complete remission from the hematologic cancer following administration of an induction therapy for the hematologic cancer that does not include alvocidib, or a prodrug thereof, or a pharmaceutically acceptable salt of the foregoing; and is MRD-negative following administration of alvocidib, or a prodrug thereof, or a pharmaceutically acceptable salt of the foregoing.

Also provided herein is a method of treating a hematologic cancer in a subject in need thereof, comprising administering to the subject a maintenance therapy comprising an effective amount of alvocidib, or a prodrug thereof, or a pharmaceutically acceptable salt of the foregoing, and one or more additional chemotherapeutic agents, wherein the subject is in complete remission from the hematologic cancer following administration of an induction therapy for the hematologic cancer that does not include alvocidib, or a prodrug thereof, or a pharmaceutically acceptable salt of the foregoing.

Also provided herein are corresponding methods of inhibiting relapse of a hematologic cancer in a subject in need thereof, converting a subject that has a hematologic cancer and is measurable residual disease (MRD)-positive for the hematologic cancer into a subject that is MRD-negative for the hematologic cancer, extending relapse-free survival of a subject having a hematologic cancer, maintaining a subject in need thereof in complete remission from a hematologic cancer, and extending progression-free survival of a subject having a hematologic cancer.

Also provided herein is a method of treating multiple myeloma in a subject in need thereof, comprising administering to the subject an effective amount of alvocidib, or a prodrug thereof, or a pharmaceutically acceptable salt of the foregoing (e.g., a compound of structural formula Ia:

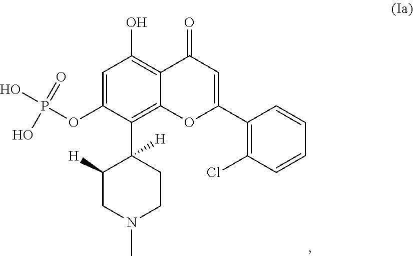

or a pharmaceutically acceptable salt thereof), wherein the subject is in complete remission from the multiple myeloma and MRD-positive following a prior maintenance therapy that includes lenalidomide, or a pharmaceutically acceptable salt thereof, and does not include alvocidib, or a prodrug thereof, or a pharmaceutically acceptable salt of the foregoing.

Also provided herein is a method of treating acute myeloid leukemia (AML) in a subject in need thereof, comprising administering to the subject an effective amount of alvocidib, or a prodrug thereof, or a pharmaceutically acceptable salt of the foregoing (e.g., a compound of structural formula Ia, or a pharmaceutically acceptable salt thereof), wherein the subject is in complete remission from the AML and MRD-positive following a prior induction therapy that includes cytarabine, or a pharmaceutically acceptable salt thereof, and daunorubicin, or a pharmaceutically acceptable salt thereof, and does not include alvocidib, or a prodrug thereof, or a pharmaceutically acceptable salt of the foregoing.

Also provided herein is a method of treating a cancer in a subject in need thereof, comprising administering to the subject a therapeutically effective amount of a therapy comprising alvocidib, or a prodrug thereof, or a pharmaceutically acceptable salt of the foregoing, wherein the subject has one or more mutations in one or more of RUNX1, SRSF2, SF3B1, U2AF1, ZRSR2, ASXL1, EZH2, BCOR and STAG2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments.

FIG. 1 is a consort diagram, and shows the design of the Phase 1 clinical trial described in Example 3.

FIG. 2 is a Swimmer's plot of best treatment response and survival for all 32 patients in the Phase 1 clinical trial described in Example 3. The swim lanes represent subjects in the study and indicate their progression and survival in the trial along with response to therapy. The horizontal axis depicts survival in months since the first dose of alvocidib. Colors of the swim lanes depict best response to treatment (orange—CR (MRD negative), blue—CR/CRi (MRD positive), green—partial remission, grey—no response). Solid black circles (●) on the swim lanes represent earliest CR/CRi or PR, black triangles (♦) represent allogenic stem cell transplantation, black circles (∘) represent relapse/progression and red stars represent death. Follow-up data (up to a protocol-specified maximum of 2 years) is still being accrued.

FIG. 3A shows the overall survival curve for all patients in the Phase 1 clinical trial described in Example 3 using Kaplan-Meier estimate (median follow-up: 9.2 months).

FIG. 3B shows the event-free survival curve from Day 1 of treatment up to death, relapse or no response to treatment for all patients in the Phase 1 clinical trial described in Example 3 using Kaplan-Meier estimate (median follow-up: 7.8 months).

FIG. 3C shows the relapse-free survival curve for all patients in the Phase 1 clinical trial described in Example 3 using Kaplan-Meier estimate, where relapse-free survival is defined as time from CR/CRi up to disease relapse or death (median follow-up: 8.7 months).

FIG. 4A is a heatmap of all patients with baseline AML mutations, and shows the frequency of baseline AML mutations determined for all responders (CR) and non-responders (No CR, includes partial responders). Colors in the legend to the right represent the percentage of patients with a mutation achieving CR or No CR. One patient who achieved a CR without detectable MRD was not assessed for the full panel of 15 genes, and was excluded from this analysis.

FIG. 4B is a heatmap of overall response versus mutation(s) for each patient in the trial of Example 3.

FIG. 5 is a box-and-whisker plot, and shows the MCL-1 scores from patients with CR (n=18) and without CR (n=9). MCL-1 scores for 4 patients with CR were not available, and are excluded. Only the response evaluable population (n=31) is included in the analysis, as one patient died prior to response evaluation.

FIG. 6 is a bar graph, and shows the MCL-1 dependency status of MDS blasts before and after treatment with azacitidine.

### DETAILED DESCRIPTION

A description of example embodiments follows.

### Methods

Provided herein are methods of treating a cancer (e.g., a hematologic cancer) in a subject in need thereof, inhibiting relapse of a cancer (e.g., a hematologic cancer) in a subject in need thereof, converting a subject that has a cancer (e.g., a hematologic cancer) and is measurable residual disease (MRD)-positive for the cancer into a subject that is MRD-negative for the cancer, extending relapse-free survival of a subject having a cancer (e.g., a hematologic cancer), maintaining a subject in need thereof in complete remission from a cancer (e.g., a hematologic cancer), and extending progression-free survival of a subject having a cancer (e.g., a hematologic cancer).

The terms “a,” “an,” “the” and similar terms used in the context of the present disclosure (especially in the context of the claims) are to be construed to cover both the singular and plural unless otherwise indicated herein or clearly contradicted by the context.

“Treating” or “treatment,” as used herein, refers to the administration of a medication or medical care to a subject, such as a human, having a disease or condition of interest, e.g., a cancer, and includes: (i) inhibiting the disease or condition, e.g., arresting its development; (ii) relieving the disease or condition, e.g., causing regression of the disease or condition; (iii) relieving the symptoms resulting from the disease or condition (e.g., pain, weight loss, cough, fatigue, weakness, etc.), and/or (iv) inhibiting relapse of the disease or condition, e.g., as that term is described herein.

“Cancer” refers to an uncontrolled growth of cells and/or abnormal increased cell survival and/or inhibition of apoptosis which interferes with the normal functioning of the bodily organs and systems. “Cancer” includes solid and non-solid cancers. A subject that has a cancer has an objectively measurable number of cancer cells present in the subject's body. “Cancers” include benign and malignant cancers (e.g., benign and malignant tumors, respectively), as well as dormant cancers and micrometastases. Carcinoma is a malignancy that begins in the skin or in tissues that line or cover internal organs. Sarcoma is a malignancy that begins in bone, cartilage, fat, muscle, blood vessels, or other connective or supportive tissue. Leukemia is a malignancy that starts in blood-forming tissue, such as the bone marrow, and causes large numbers of abnormal blood cells to be produced and enter the blood. Lymphoma and multiple myeloma are malignancies that begin in the cells of the immune system. Central nervous system cancers are malignancies that begin in the tissues of the brain and spinal cord.

The term “solid tumor,” as used herein, refers to malignancies/cancers formed of abnormal masses of tissue that usually do not contain cysts or liquid areas. Solid tumors are named/classified according to the tissue/cells of origin. Examples include, but are not limited to, sarcomas and carcinomas.

The term “leukemia,” as used herein, refers to hematologic or blood cell malignancies/cancers that begin in blood-forming tissue, such as the bone marrow. Examples include, but are not limited to, chronic leukemia, acute leukemia, acute myeloid leukemia (AML), chronic myeloid leukemia (CML), acute lymphocytic leukemia (ALL), acute lymphoblastic leukemia (e.g., B-cell, T-cell) and chronic lymphocytic leukemia (CLL).

The term “lymphoma,” as used herein, refers to lymphatic cell malignancies/cancers that begin in the cells of the immune system. Examples include, but are not limited to, non-Hodgkin's lymphoma and multiple myeloma.

“Metastasis” refers to the spread of cancer from its primary site to other places in the body. “Metastases” are cancers which migrate from their original location and seed vital organs, which can eventually lead to the death of the subject through the functional deterioration of the affected organs. Metastasis is a sequential process, where cancer cells can break away from a primary tumor, penetrate into lymphatic and blood vessels, circulate through the bloodstream, and grow in a distant focus (metastasize) in normal tissues elsewhere in the body. At the new site, the cells establish a blood supply and can grow to form a life-threatening mass. Metastasis can be local or distant. Both stimulatory and inhibitory molecular pathways within the tumor cell regulate this behavior, and interactions between the tumor cell and host cells in the new site are also significant.

A wide variety of cancers, including solid tumors, leukemias and lymphomas are amenable to the methods disclosed herein. In some embodiments, the cancer is a solid cancer. In some embodiments, the cancer comprises a solid tumor (e.g., a colorectal, breast, prostate, lung, pancreatic, renal or ovarian tumor). Accordingly, in some embodiments, the cancer is a solid tumor cancer. In various embodiments, the solid tumor cancer is breast cancer, bladder cancer, liver cancer, pancreatic cancer, lung cancer, colorectal cancer, ovarian cancer, prostate cancer, or melanoma. In some embodiments, the cancer is bladder cancer. In some embodiments, the cancer is lung cancer (e.g., non-small cell lung cancer). In other embodiments, the cancer is liver cancer. In some embodiments, the cancer is a sarcoma, bladder cancer or renal cancer. In some embodiments, the cancer is prostate cancer (e.g., castration-resistant prostate cancer, castration-sensitive prostate cancer). In other embodiments, the cancer is bladder cancer, pancreatic cancer, colorectal cancer, glioblastoma, kidney cancer, non-small cell lung carcinoma, prostate cancer, sarcoma, skin cancer, thyroid cancer, testicular cancer or vulvar cancer. In some embodiments, the cancer is endometrial cancer, pancreatic cancer, testicular cancer, renal cancer, melanoma, colorectal cancer, thyroid cancer, bladder cancer, pancreatic cancer, vulvar cancer, sarcoma, prostate cancer, lung cancer or anal cancer. In some embodiments, the cancer is a sarcoma. In some embodiments, the cancer is a renal cell carcinoma.

In some embodiments, the cancer is a hematologic cancer. Hematologic cancers that can be treated according to the methods described herein include leukemias (e.g., acute leukemias, chronic leukemias), lymphomas (e.g., B-cell lymphoma, T-cell lymphoma) and multiple myeloma. In some embodiments, the hematologic cancer is selected from multiple myeloma, myelodysplastic syndrome (MDS), acute myeloid leukemia (AML), acute lymphoblastic leukemia (ALL), acute lymphocytic leukemia, lymphocytic lymphoma, mycosis fungoides, chronic lymphogenous leukemia, chronic lymphocytic leukemia (CLL), mantle cell lymphoma, diffuse large B-cell lymphoma, follicular lymphoma, or non-Hodgkin's lymphoma.

In some embodiments, the cancer is AML (e.g., secondary AML, therapy-related AML, AML associated with MDS-related changes, AML associated with the genomic/genetic signature described in Lindsley et al. or a genomic/genetic signature described herein, newly diagnosed AML, particularly newly-diagnosed AML in elderly patients). Lindsley, R. C., et al., Blood 26 Feb. 2015, Volume 125, No. 9, 1367-76 identified a genomic/genetic signature specific for secondary AML. Lindsley et al. showed that the presence of a mutation in any one or more of SRSF2, SF3B1, U2AF1, ZRSR2, ASXL1, EZH2, BCOR and STAG2 was >95% specific for secondary AML. This so-called genetic signature of secondary AML is shared by therapy-related AML and elderly de novo AML populations, and is associated with a subset of AML patients with worse clinical outcomes, including a lower CR rate, more frequent re-induction and decreased, event-free survival. Lindsley et al. Mutations in RUNX1 and/or ASXL1, particularly when unaccompanied by favorable-risk genetics (e.g., t(8;21)(q22;q22.1); RUNX1-RUNX1T1 inv(16)(p13.1q22) or t(16;16)(p13.1;q22); CBFB-MYH11; mutated NPM1 without FLT3-ITD or with FLT3-ITD^{low}; biallelic mutated CEBPA), are associated with adverse-risk AML according to ELN risk stratification guidelines in recognition of their independent association with adverse risk. RUNX1 mutations, for example, are associated with poor prognosis, and ASXL1 mutations with inferior survival. Dohner, H., et al., Blood 26 Jan. 2017; Vol. 129; No. 4; 424-447.

In some embodiments, a subject (e.g., a subject having an AML described herein) has a mutation (e.g., one or more mutations) in one or more (e.g., one, at least two, two, at least three, three, at least four, four, at least five, five, at least six, six, at least seven, seven, at least eight, eight, nine) of RUNX1, SRSF2, SF3B1, U2AF1, ZRSR2, ASXL1, EZH2, BCOR and STAG2. In some embodiments, a subject has a mutation (e.g., one or more mutations) in NPM1. In some embodiments, a subject has a mutation (e.g., one or more mutations) in one or more (e.g., one, at least two, two, at least three, three, at least four, four, at least five, five, at least six, six, at least seven, seven, at least eight, eight, at least nine, nine, ten) of RUNX1, SRSF2, SF3B1, U2AF1, ZRSR2, ASXL1, EZH2, BCOR, STAG2 and NPM1. Non-limiting examples of mutation patterns include mutation(s) in RUNX1; SRSF2; SF3B1; U2AF1; ZRSR2; ASXL1; EZH2; BCOR; STAG2; NPM1; SRSF2 and BCOR; IDH2, SRSF2 and BCOR; NPM1; NPM1, IDH1 and NRAS; FLT3; CEBPA; ASXL1 and TET2; RUNX1, IDH1, SRSF2 and BCOR; RUNX1, SRSF2 and BCOR; RUNX1, IDH2 and SRSF2; RUNX1 and SRSF2; TP53; U2AF1 and BCOR; DNMT3A, IDH1 and NPM1; NPM1 and DNMT3A; NPM1 and TET2; NPM1, DNMT3A and NRAS; NPM1, FLT3, CEBPA, DNMT3A; ASXL1, RUNX1, EZH2, IDH2 and NRAS; ASXL1, RUNX1 and EZH2; FLT3, ASXL1, RUNX1 and BCOR; and ASXL1, RUNX1 and BCOR.

In some embodiments, the cancer is a pre-metastatic cancer. In some embodiments, the cancer is a metastatic cancer.

“Frontline,” as used herein, refers to a cancer that has not previously been treated with a traditional therapy, such as radiation, surgery or chemotherapy. Frontline cancers are also sometimes referred to as newly-diagnosed. In some embodiments of the methods described herein, the cancer is frontline or previously untreated (e.g., prior to a prior therapy or induction therapy).

In some embodiments, the cancer is relapsed and/or refractory.

As used herein, the terms “relapse” and “relapsed” have their ordinary meaning in the art, and may refer to the return of a cancer or the signs and symptoms of the cancer after a period of complete remission (e.g., initial complete remission) due to treatment. In some embodiments of the methods described herein, the cancer is relapsed. In some embodiments, relapse may refer to the recurrence of cancer after complete remission meeting one or more of the following criteria (i) ≥5% blasts in the marrow or peripheral blood, (ii) extramedullary disease, and/or cancer presence determined by a physician upon clinical assessment.

As used herein, the term “refractory” has its ordinary meaning in the art, and may refer to a cancer that does not respond to treatment. It includes cancers that are refractory at the beginning of treatment and cancers that become refractory during treatment (as by the emergence of resistance, for example). In some embodiments of the methods described herein, the cancer is refractory.

Examples of cancer treatable according to the methods described herein include, but are not limited to, adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional examples of cancer treatable according to the methods described herein include, but are not limited to, histiocytic disorders; leukemia; histiocytosis malignant; Hodgkin's disease; hypereosinophilia, immunoproliferative small; non-Hodgkin's lymphoma; plasmacytoma; reticuloendotheliosis; melanoma; chondroblastoma; chondroma; chondrosarcoma; dermatofibrosarcoma protuberans, fibrotic cancer; fibroma; fibrosarcoma; giant cell tumors; histiocytoma; lipoma; liposarcoma; mesothelioma; myxoma; myxosarcoma; osteoma; osteosarcoma; pediatric malignancy, chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatocellular cancer, hepatoma; hidradenoma; islet cell tumor; Leydig cell tumor; papilloma; sertoli cell tumor; theca cell tumor; leiomyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. Yet more examples of cancer treatable according to the methods described herein include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Further examples of cancers treatable according to the methods described herein include, but are not limited to, Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Cancer of the anal region; Anal Cancer; Appendix Cancer; Astrocytomas, Childhood; Atypical Teratoid/Rhabdoid Tumor, Childhood, Central Nervous System (CNS); Neoplasms of the CNS (e.g., primary CNS lymphoma, spinal axis tumors, medulloblastoma, brain stem gliomas or pituitary adenomas), Barrett's esophagus (e.g., pre-malignant syndrome), and mycoses fungoides, Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sézary Syndrome); Ductal Carcinoma In Situ (DCIS); Embryonal Tumors, Central Nervous System, Childhood; Cancer of the Endocrine system (e.g., cancer of the thyroid, pancreas, parathyroid or adrenal glands), Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Gynecologic Tumors ((e.g., uterine sarcomas, carcinoma of the fallopian tubes, carcinoma of the endometrium, carcinoma of the cervix, carcinoma of the vagina or carcinoma of the vulva), Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Cutaneous or Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CIVIL); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sézary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis (e.g., renal cell carcinoma, carcinoma of the renal pelvis), benign prostatic hypertrophy, parathyroid cancer, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein.

“Subject” includes humans, domestic animals, such as laboratory animals (e.g., monkeys, rats, mice, etc.), household pets (e.g., cats, dogs, rabbits, etc.), and livestock (e.g., pigs, cattle, sheep, goats, horses, etc.), and non-domestic animals (e.g., bears, elephants, porcupines, etc.). In embodiments, a subject is a mammal. In embodiments, a subject is a human.

There are many factors that can affect treatment outcomes in subjects being treated for cancer. For example, age, fitness for chemotherapy, transplant eligibility and MCL-1 dependence have all been linked to treatment outcomes in subjects being treated for cancer. Accordingly, in some embodiments of the methods disclosed herein, the subject is young (i.e., aged less than 60 years). In some embodiments, the subject is elderly (i.e., aged 60 years or more).

In some embodiments, the subject is fit. In some embodiments, the subject is unfit. As used herein, “unfit” refers to having one or more physiological impairments that render a subject ineligible for a certain treatment (e.g., standard-of-care chemotherapy, intensive chemotherapy, non-intensive chemotherapy). Some have taken a consensus-based approach to determining fitness of a subject. See, for example, Ferrara, F., et al., Leukemia (2013) 27, 997-999, the relevant teachings of which are incorporated herein by reference in their entireties. In some embodiments, fitness may be determined by the consensus approach put forth in Ferrara, F., et al. In such embodiments, unfitness to intensive chemotherapy means fulfillment of at least one of nine criteria identified in Ferrara, F., et al., and unfitness to non-intensive chemotherapy means fulfillment of at least one of six criteria identified in Ferrara, F., et al. The Eastern Cooperative Oncology Group (ECOG) has put forth the ECOG Performance Status, which is a tool used to quantify the functional status of cancer patients on a scale of 0-5. In some embodiments, fitness may be determined by the ECOG Performance Status. In some embodiments, an ECOG score of greater than or equal to 2 (e.g., greater than 2) renders a subject unfit.

A “fit” subject is a subject that is not unfit.

In some embodiments, the subject is young and/or fit. In some embodiments, the subject is elderly and/or unfit. In some embodiments, the subject is young and/or unfit. In some embodiments, the subject is elderly and/or fit.

In some embodiments, the subject is transplant ineligible (e.g., according to one or more established guidelines). In some embodiments, the subject is transplant eligible (e.g., according to one or more established guidelines).

Various organizations have issued guidelines on assigning patients as transplant ineligible and/or transplant eligible. For example, National Comprehensive Cancer Network (NCCN), European LeukemiaNet (ELN) and European Society of Medical Oncology (ESMO) have issued guidelines for transplant eligibility in AML. NCCN's guidelines state that transplant should be considered only if a patient has entered remission or in the context of clinical trial. Transplant-based options (either matched sibling or alternative donor allogeneic hematopoietic cell transplantation (HCT)) afforded a lower risk of relapse and somewhat higher disease-free survival (DFS) when given as consolidation for patients with intermediate-risk cytogenetics. For favourable-risk patients, if MRD is positive, or they have an identified sibling or alternative donor, transplant can be considered. Allogenic transplant should be reserved for patients who have persistent disease despite therapy for relapsed disease. See https://www.nccn.org/professionals/physician_gls/pdf/aml.pdf. ELN's guidelines state that comorbidity predicts post transplantation outcome. The HCT-comorbidity index (CI) was developed as an instrument that captures pre-transplantation comorbidities and can be used in predicting post transplantation outcomes and stratifying patients with AML. Disease risk scored according to the International Prognostic Scoring System (IPSS), age, and presence of comorbidity graded according to the HCT-CI were recognized as the most relevant clinical variables to be considered in order to judge a patient eligible for allogeneic stem cell transplantation (SCT). Fit patients up to age 65 to 70 years with IPSS intermediate-2 or high risk and those with IPSS intermediate-1 risk with excess blasts or poor-risk cytogenetics are candidates for allogeneic SCT. See https://ashpublications.org/blood/article/129/4/424/36196/Diagnosis-and-management-of-AML-in-adults-2017-ELN. ESMO's guidelines state that the HCT-CI score predicts treatment-related mortality in patients treated with induction chemotherapy, as well as transplant outcome. In CR1, alloHCT is indicated in patients with intermediate or adverse risk AML and age ≤75. BCR-ABL-positive patients should undergo alloHCT as soon as they achieve remission. In older patients, comorbidities must be carefully evaluated. In this regard, the HCT-CI predicts nonrelapse mortality and OS and helps in the selection of patients. Likewise, the combination of HCT-CI with the European Society for Blood and Marrow Transplantation score translates into nonrelapse mortality prediction for patients undergoing reduced-intensity conditioning alloHCT in CR1. Risk factors for adverse long-term effects after alloHCT include pre-existing comorbidities, pretransplant therapy, type of transplant conditioning regimen, stem cell source, donor characteristics, GvHD occurrence and treatment. See https://www.esmo.org/guidelines/haematological-malignancies/acute-myeloid-leukaemia.

In some embodiments, a subject having AML is transplant ineligible or transplant eligible according to NCCN guidelines. In some embodiments, a subject having AML is transplant ineligible or transplant eligible according to ELN guidelines. In some embodiments, a subject having AML is transplant ineligible or transplant eligible according to ESMO guidelines.

For example, NCCN and ESMO have issued guidelines for transplant eligibility in ALL. NCCN's guidelines recommend bone marrow transplant (HCT) over chemotherapy alone for relapsed/refractory ALL patients, but many patients are not eligible based on age or progression of the disease. As a post-remission consolidation therapy, the decision to proceed with allogeneic/autologous HCT, or prolonged maintenance are mutually exclusive approaches in ALL therapy. Pre-treatment with CAR-T cells act as a bridge for transplant, and patients who were formerly ineligible for transplant due to poor remission status have a CR and can be transplanted. See https://www.nccn.org/professionals/physician_gls/pdf/all.pdf. ESMO guidelines reflect the general agreement that SCT is clearly the best therapeutic option for patients in second or later CR. Following are the recommendations for SCT in adult ALL:
1. On Complete remission 1: AutoSCT versus nontransplantation
a) AutoSCT not recommended outside a clinical trial
b) Maintenance therapy, biological therapy, or TKIs may improve outcomes in selected patients
2. On Complete remission 1: AlloSCT versus nontransplantation
a) AlloSCT recommended in all patients with poor early MRD response
b) AlloSCT not recommended in SR patients with sustained molecular response
c) Indication unclear in HR patients with sustained molecular response
3. Complete remission ≥2: AlloSCT superior to non-transplantation
4. AlloSCT versus autoSCT
a) Advantage for alloSCT
b) Insufficient data in patients with negative MRD levels, including Ph+ ALL
5. Sibling donor versus matched unrelated donor (MUD): Similar, and possibly equivalent survival outcomes
6. Unrelated donor (UD) cord blood transplantation (CBT) versus UD bone marrow transplantation (BMT):
a) Consider CBT if no HLA-well-matched donor or need for urgent SCT
b) Haploidentical SCT should also be considered in this setting
7. Conditioning regimens:
a) Benefit of total body irradiation (TBI) regimens for myeloablative SCT
b) Reduced intensity conditioning (MC) regimens appropriate only for adults in remission unfit for myeloablative conditioning and elderly fit patients. See https://www.esmo.org/guidelines/haematological-malignancies/acute-lymphoblastic-leukaemia.

In some embodiments, a subject having ALL is transplant ineligible or transplant eligible according to NCCN guidelines. In some embodiments, a subject having ALL is transplant ineligible or transplant eligible according to ESMO guidelines.

For example, NCCN and ESMO have issued guidelines for transplant eligibility in CLL. NCCN's guidelines state that allogeneic HCT can be considered for CLL refractory to small molecule inhibitor therapy in patients without significant comorbidities. HCT-CI could be used for the assessment of comorbidities prior to HCT and to predict the risk of non-relapse mortality and the probabilities of survival after HCT. See https://www.nccn.org/professionals/physician_gls/pdf/cll.pdf. ESMO guidelines state that autologous stem-cell transplantation is not useful in CLL. An alloSCT should be considered in patients achieving remission with kinase inhibitors or BCL2 antagonists after early relapse from chemoimmunotherapy and/or with del(17p) or TP53 mutation. In this situation, long-term treatment with inhibitors is an alternative option. The decision should be based on transplant- and disease-risk (e.g., availability of matched donor, patient age and comorbidities and response to treatment) and the patient's preferences, following a careful discussion of the risks and benefits of an allogeneic transplant. In patients failing several lines of therapy, allogeneic bone marrow transplantation should be considered. See https://www.esmo.org/guidelines/haematological-malignancies/chronic-lymphocytic-leukaemia.

In some embodiments, a subject having CLL is transplant ineligible or transplant eligible according to NCCN guidelines. In some embodiments, a subject having CLL is transplant ineligible or transplant eligible according to ESMO guidelines.

For example, NCCN, American Society of Clinical Oncology (ASCO), National Institute for Health and Care Excellence (NICE) and ESMO have issued guidelines for transplant eligibility in MM. NCCN's guidelines state that age and comorbidities should be considered when evaluating patients for stem cell transplant. However, advanced age and renal dysfunction are not absolute contradiction to transplant. See https://www.nccn.org/professionals/physician_gls/pdf/myeloma.pdf. ASCO's guidelines state that chronologic age and renal function should not be the sole criteria used to determine eligibility for SCT. Multiple factors should be considered: disease-specific factors such as stage and cytogenetic abnormalities, and patient-specific factors including age, comorbidities, functional status, frailty status, and patient preferences. See https://ascopubs.org/doi/pdf/10.1200/JCO.18.02096. NICE's guidelines suggest using frailty and performance status measures that include comorbidities to assess the suitability of people with myeloma for first autologous stem cell transplant, and not using age or the level of renal impairment alone to assess the suitability of people with myeloma for first autologous stem cell transplant. Non-limiting examples of factors for allogeneic transplantation include:
1. Whether the person has chemosensitive disease
2. How many previous lines of treatment a person has had
3. Whether a fully human leukocyte antigen (HLA) matched donor is available
4. How graft versus host disease (GvHD) and other complications may get worse with age
5. The risk of higher transplant-related mortality and morbidity, versus the potential for long-term disease-free survival
6. Improving outcomes with other newer treatments
7. The person's understanding of the procedure and its risks and benefits.

See https://www.nice.org.uk/guidance/ng35/chapter/Recommendations. ESMO's guidelines state that for patients <70 years in good clinical condition, induction followed by high-dose therapy with ASCT is the standard treatment. See https://www.esmo.org/guidelines/haematological-malignancies/multiple-myeloma.

In some embodiments, a subject having MM is transplant ineligible or transplant eligible according to NCCN guidelines. In some embodiments, a subject is transplant eligible or transplant ineligible according to ASCO guidelines. In some embodiments, a subject having MM is transplant eligible or transplant ineligible according to NICE guidelines. In some embodiments, a subject having MM is transplant ineligible or transplant eligible according to ESMO guidelines.

For example, NCCN, ELN and ESMO have issued guidelines for transplant eligibility in MDS. NCCN's guidelines state that transplant eligibility principles include patients having fit performance status, age, and having a donor. The HCT-CI can be used to evaluate the significance of comorbidities on survival outcomes of patients. See https://www.nccn.org/professionals/physician_gls/pdf/mds.pdf. ELN's guidelines state that the assessment of individual risk enables the identification of fit patients with a poor prognosis who are candidates for up-front intensive treatments, primarily allogeneic stem cell transplantation. Comorbidity predicts posttransplantation outcome. HCT-CI is an instrument that captures pretransplantation comorbidities and can be used in predicting posttransplantation outcomes and stratifying patients with MDS. See https://ashpublications.org/blood/article-lookup/doi/10.1182/blood-2013-03-492884. ESMO's guidelines state that the major obstacle to alloSCT is the fact that most MDS patients are above the age of 70 years. Co-morbidity, age, IPSS and IPSS-R score, cytogenetics, conditioning regimen and donor selection are predictors of post-transplant outcome and should be taken into account carefully during the decision process. All patients diagnosed with higher-risk MDS aged <65-70 years (although particularly ‘fit’ patients aged >70 years may sometimes be considered) should be evaluated for alloSCT eligibility. HLA-identical (or single antigen mismatched) siblings or matched unrelated individuals should be considered as suitable donors. See https://www.annalsofoncology.org/article/S0923-7534(19)34080-3/pdf.

In some embodiments, a subject having MDS is transplant ineligible or transplant eligible according to NCCN guidelines. In some embodiments, a subject having MDS is transplant eligible or transplant ineligible according to ELN guidelines. In some embodiments, a subject having MDS is transplant ineligible or transplant eligible according to ESMO guidelines.

In some embodiments, the cancer (e.g., hematologic cancer) is MCL-1 dependent. As used herein, “MCL-1-dependent” refers to the subset of cancers wherein myeloid cell leukemia 1 (MCL-1) is the primary driver of suppressing apoptosis. Typically, MCL-1 dependency promotes AML blast survival, and is associated with treatment resistance and relapse. MCL-1 dependence can be assessed, for example, by contacting a subject's cancer cell with a profiling peptide, as described in International Publication Nos. WO 2016/172214 and WO 2018/119000, the relevant contents of which are incorporated herein by reference in their entireties. Examples 4-6 herein describe the assessment of MCL-1 dependence in various populations of hematologic cancer cells, including blasts, from MM, MDS, AML and ALL patient samples.

Although not wishing to be bound by any particular theory, it is thought that MCL-1 dependence is found in both MRD cells and leukemia stem cells (LSCs), such as ALL and AML stem cells. In addition, MRD cells and LSCs are thought to be responsible for relapse in subjects and to play a role in refractory disease. Research shows that knockout of MCL-1 in mice results in loss of early bone marrow progenitor cell populations, suggesting that MCL-1 is the primary survival signal in hematopoietic stem cells. Opferman, J. T., et al., “Obligate Role of Anti-Apoptotic MCL-1 in the Survival of Hematopoietic Stem Cells,” Science, vol. 307, 18 Feb. 2005, the relevant contents of which are incorporated herein in their entireties. MCL-1 has also been identified as the main survival mechanism in LSCs from FLT3 positive AML. Yoshimoto, G., et al., “FLT3-ITD up-regulates MCL-1 to promote survival of stem cells in acute myeloid leukemia via FLT3-ITD-specific STATS activation,” Blood, vol. 114, no. 24, 3 Dec. 2009, the relevant contents of which are incorporated herein in their entireties. It is likely that all LSCs, including non-FLT3-positive LSCs, use a similar MCL-1-dependent survival mechanism as that observed in both hematopoietic stem cells generally and FLT3-positive LSCs.

Leukemia stem cells and MRD cells are not completely synonymous with one another. However, the MRD cells that ultimately lead to relapse are leukemia stem cells. See Al-Malawi, A., “Leukemic Stem Cells Shows the Way for Novel Target of Acute Myeloid Leukemia Therapy,” J. Stem Cell Res. Ther., vol. 3, issue 4; Yanagisawa, B., et al., “Translating leukemia stem cells into the clinical setting: Harmonizing the heterogeneity,” Experimental Hematology 2016; 44: 1130-1137; and Gerber, J. M., et al., “A clinically relevant population of leukemic CD34⁺CD38⁻ cells in acute myeloid leukemia,” Blood, 12 Apr. 2012, vol. 119, no. 15, the relevant contents of which are incorporated herein in their entireties. Without wishing to be bound by any particular theory, it is thus thought that MCL-1 regulation may be a rational therapeutic strategy for cancer (e.g., a hematologic cancer).

Cyclin-dependent kinases, or CDKs, are a family of proteins that form complexes involved in either cell cycle progression or transcription regulation. CDK9 is a transcription-regulating CDK that promotes the expression of MCL-1 by phosphorylating the carboxyl-terminal domain of the largest subunit of RNA polymerase II, allowing transcriptional elongation of MCL-1 mRNA. Inhibition of CDK9, as by a CDK9 inhibitor such as alvocidib, or a prodrug thereof, or a pharmaceutically acceptable salt of the foregoing, is thus thought to provide MCL-1 regulation that could be used to eliminate or substantially eliminate MCL-1-dependent cells, such as MRD cells and LSCs, thereby treating a cancer (e.g., a hematologic cancer) in a subject in need thereof, inhibiting relapse of a cancer (e.g., a hematologic cancer) in a subject in need thereof, converting a subject that has a cancer (e.g., a hematologic cancer) and is MRD-positive for the cancer into a subject that is MRD-negative for the cancer, extending relapse-free survival of a subject having a cancer (e.g., a hematologic cancer), maintaining a subject in need thereof in complete remission from a cancer (e.g., a hematologic cancer), and/or extending progression-free survival of a subject having a cancer (e.g., a hematologic cancer).

As used herein, “inhibiting relapse” refers to the administration of a medication or medical care to a subject, such as a human, in complete remission from a cancer, and includes: (i) extending relapse-free survival from a cancer (e.g., beyond the mean and/or median time of relapse-free survival associated with a particular cancer and therapeutic regimen), (ii) converting a subject that is MRD-positive for a cancer into a subject that is MRD-negative for the cancer, (iii) maintaining a subject in complete remission from a cancer for a particular period of time (e.g., for at least 3 months, at least 6 months, at least 9 months, at least 12 months, etc. from when the subject is determined to be in complete remission), and (iv) extending progression-free survival from a cancer (e.g., beyond the mean and/or median time of progression-free survival associated with a particular cancer and therapeutic regimen).

As used herein, “relapse-free survival” refers to the length of time after treatment for a disease, such as cancer, ends that a subject survives without clinically relevant signs or symptoms of that disease. In clinical trials, relapse-free survival, or RFS, can be reported for a cohort or population of patients, typically, as a median for the cohort or population.

As used herein, “progression-free survival” refers to the length of time during and after the treatment of a disease, such as cancer, that a subject lives with the disease, and it does not get worse. In clinical trials, progression-free survival, or PFS, can be reported for a cohort or population of patients, typically, as a median for the cohort or population.

In some embodiments, the method comprises administering to the subject an effective amount of alvocidib, or a prodrug thereof, or a pharmaceutically acceptable salt of the foregoing, and the subject is in complete remission from the cancer (e.g., hematologic cancer) and MRD-positive following administration of a prior therapy (e.g., a prior therapy that does not include alvocidib, or a prodrug thereof, or a pharmaceutically acceptable salt of the foregoing). In some embodiments, the method comprises administering to the subject an effective amount of alvocidib, or a prodrug thereof, or a pharmaceutically acceptable salt of the foregoing, and the subject is in complete remission from the cancer (e.g., hematologic cancer) and MRD-positive following administration of a prior therapy (e.g., a prior therapy that does not include alvocidib, or a prodrug thereof, or a pharmaceutically acceptable salt of the foregoing); and is MRD-negative following administration of alvocidib, or a prodrug thereof, or a pharmaceutically acceptable salt of the foregoing.

In some embodiments, the method comprises administering to the subject a maintenance therapy comprising an effective amount of alvocidib, or a prodrug thereof, or a pharmaceutically acceptable salt of the foregoing, and the subject is in complete remission from the cancer (e.g., hematologic cancer) following administration of an induction therapy (e.g., an induction therapy for the cancer that does not include alvocidib, or a prodrug thereof, or a pharmaceutically acceptable salt of the foregoing). In some embodiments, the subject is MRD-positive following the induction therapy. In some embodiments, the subject is MRD-negative following administration of alvocidib, or a prodrug thereof, or a pharmaceutically acceptable salt of the foregoing.

In some embodiments, the method comprises treating a cancer (e.g., hematologic cancer) in a subject in need thereof, comprising administering to the subject a maintenance therapy comprising an effective amount of alvocidib, or a prodrug thereof, or a pharmaceutically acceptable salt of the foregoing, and one or more additional chemotherapeutic agents, wherein the subject is in complete remission from the hematologic cancer following administration of an induction therapy for the hematologic cancer (e.g., an induction therapy that does not include alvocidib, or a prodrug thereof, or a pharmaceutically acceptable salt of the foregoing).

Alvocidib, or a prodrug thereof, or a pharmaceutically acceptable salt of the foregoing, used in the maintenance therapy contexts described herein (e.g., as a maintenance therapy, following administration of a prior therapy, such as a prior therapy that does not include alvocidib, or a prodrug thereof, or a pharmaceutically acceptable salt of the foregoing), can be employed in at least three ways: (1) as an addition to a maintenance therapy (e.g., a therapy approved by FDA as a maintenance therapy, such as lenalidomide for MM); (2) as an addition to a continuous therapy intended to bring about complete remission (e.g., a continuous therapy not approved by FDA as a maintenance therapy as such), upon achievement of complete remission in the subject to maintain the complete remission; and (3) as an independent therapy (e.g., monotherapy, combination therapy) following completion of a prior and/or induction therapy (e.g., a non-continuous prior and/or induction therapy, such as VYXEOS®), upon achievement of complete remission in the subject to maintain the complete remission.

In each of the contexts described above, administration of alvocidib, or a prodrug thereof, or a pharmaceutically acceptable salt of the foregoing, follows a prior therapy or induction therapy. “Follows,” in these contexts, means that the subject has received at least one dose (e.g., has completed one treatment regimen, one cycle) of the prior therapy or induction therapy prior to being administered alvocidib, or a prodrug thereof, or a pharmaceutically acceptable salt of the foregoing. “Follows,” particularly in the contexts wherein alvocidib, or a prodrug thereof, or a pharmaceutically acceptable salt of the foregoing, is added to a maintenance therapy or continuous therapy intended to bring about complete remission, does not necessarily (and, typically, does not) imply that the prior therapy has been terminated prior to administration of alvocidib, or a prodrug thereof, or a pharmaceutically acceptable salt of the foregoing. Often, alvocidib, or a prodrug thereof, or a pharmaceutically acceptable salt of the foregoing, will be added to the prior therapy or induction therapy without interrupting the continuity of the prior therapy.

When alvocidib, or a prodrug thereof, or a pharmaceutically acceptable salt of the foregoing, is used as an independent therapy following completion of a prior therapy or induction therapy, however, administration of alvocidib, or a prodrug thereof, or a pharmaceutically acceptable salt of the foregoing, will begin after the prior therapy or induction therapy has been discontinued. In this context, administration of alvocidib, or a prodrug thereof, or a pharmaceutically acceptable salt of the foregoing, can begin immediately after the prior therapy or induction therapy has been discontinued, or can be separated from the completion of the prior therapy or induction therapy in time and/or by an intervening therapy.

When a subject is described, for example, as being in complete remission or MRD-positive, following administration of a prior therapy or induction therapy, “following” is intended to encompass all of the contexts described above, such that at the time the subject's status is being assessed, the subject may have received one dose (e.g., completed one treatment regimen, one cycle) of the prior therapy or induction therapy (e.g., as when alvocidib, or a prodrug thereof, or a pharmaceutically acceptable salt of the foregoing, is being used as an addition to a maintenance therapy or continuous therapy intended to bring about complete remission), or may have had the prior therapy or induction therapy discontinued (e.g., as when alvocidib, or a prodrug thereof, or a pharmaceutically acceptable salt of the foregoing, is being used as an independent therapy following completion of a prior therapy or induction therapy).

Also provided herein are methods comprising administering to the subject a therapeutically effective amount of alvocidib, or a prodrug thereof, or a pharmaceutically acceptable salt of the foregoing (e.g., as a first-line and/or induction therapy), wherein the subject has one or more mutations in one or more of RUNX1, SRSF2, SF3B1, U2AF1, ZRSR2, ASXL1, EZH2, BCOR and STAG2. In some embodiments, the method comprises administering to the subject a therapeutically effective amount of a therapy comprising alvocidib, or a prodrug thereof, or a pharmaceutically acceptable salt of the foregoing (e.g., as a first-line and/or induction therapy), wherein the subject has one or more mutations in one or more of RUNX1, SRSF2, SF3B1, U2AF1, ZRSR2, ASXL1, EZH2, BCOR and STAG2.

In some embodiments, the method comprises determining whether a subject has one or more mutations in one or more of RUNX1, SRSF2, SF3B1, U2AF1, ZRSR2, ASXL1, EZH2, BCOR and STAG2; and administering a therapeutically effective amount of alvocidib, or a prodrug thereof, or a pharmaceutically acceptable salt of the foregoing (e.g., as a first-line and/or induction therapy) to the subject (e.g., if the subject is determined to have one or more mutations in one or more of RUNX1, SRSF2, SF3B1, U2AF1, ZRSR2, ASXL1, EZH2, BCOR and STAG2).

Methods of conducting mutation analyses, e.g., to determine whether a subject has one or more mutations, are known in the art, and include next generation sequencing. For example, institutions use commercially available products and reagents to establish their own molecular pathology analytic processes. The combined cancer panel from one institution, for example, targets exonic and intronic sequences obtained from DNA purified from tumor (with or without normal DNA) using Custom Agilent SureSelect capture and Illumina HiSeq2500 sequencing. Samples have an average coverage of at least 500-fold, and at least 30-fold coverage of greater than 98% of coding sequences in the region of interest. These sequences are evaluated for single nucleotide variants, and small insertions and deletions. Actionable mutations are confirmed by an orthologous method. In addition, several companies, including Hematologics, Inc. and Foundation Medicine, provide commercial mutation analysis services. There are also many commercial products, including FoundationOne® Heme (available from Foundation Medicine, Cambridge, Mass.), for performing comprehensive genomic profiling.

In some embodiments, the method comprises providing a subject having one or more mutations in one or more of RUNX1, SRSF2, SF3B1, U2AF1, ZRSR2, ASXL1, EZH2, BCOR and STAG2; and administering to the subject a therapeutically effective amount of alvocidib, or a prodrug thereof, or a pharmaceutically acceptable salt of the foregoing (e.g., as a first-line and/or induction therapy).

As used herein, “therapy” refers to any drug-based cancer treatment (e.g., chemotherapy, immunotherapy, targeted therapy, hormone therapy), except a drug-based cancer treatment comprising venetoclax, or a pharmaceutically acceptable salt thereof. In some embodiments, a therapy is a chemotherapy.

“Maintenance therapy,” as used herein, means a therapy, as that term is used herein, designed/intended to maintain a subject in complete remission following an induction therapy. In some embodiments, a maintenance therapy includes at least one (e.g., one, two, three or all) therapeutic agent from the induction therapy. Non-limiting examples of maintenance therapies, e.g., for AML, include alvocidib, or a prodrug thereof, or a pharmaceutically acceptable salt of the foregoing, in the absence of an additional chemotherapeutic agent or in combination with a HMA (e.g., an oral HMA). Non-limiting examples of maintenance therapies, e.g., for MM, include alvocidib, or a prodrug thereof, or a pharmaceutically acceptable salt of the foregoing, in the absence of an additional chemotherapeutic agent or in combination with one or more of lenalidomide, bortezomib, thalidomide and carfilzomib.

A maintenance therapy can follow an induction therapy directly, or can be separated from an induction therapy in time and/or by an intervening therapy. For example, when alvocidib, or a prodrug thereof, or a pharmaceutically acceptable salt of the foregoing, is administered to a subject (e.g., a transplant-ineligible MM patient) in combination with lenalidomide maintenance therapy, the alvocidib, or a prodrug thereof, or a pharmaceutically acceptable salt of the foregoing, can be administered beginning about nine months after the lenalidomide maintenance therapy commenced, e.g., once it is clear that the subject is resistant to the lenalidomide maintenance therapy, as when the lenalidomide maintenance therapy fails to convert the subject to MRD-negative status. When alvocidib, or a prodrug thereof, or a pharmaceutically acceptable salt of the foregoing, is administered to a subject (e.g., an AML patient), e.g., as the first maintenance therapy following an induction therapy, such as induction therapy with VYXEOS®, the alvocidib, or a prodrug thereof, or a pharmaceutically acceptable salt of the foregoing, can be administered either directly following the induction therapy with VYXEOS® (e.g., as in the case of unfit and/or elderly AML patients) or following a transplant that followed the induction therapy with VYXEOS® (e.g., as in the case of fit and/or young AML patients).

“Induction therapy,” as used herein, means a therapy, as that term is used herein, but not necessarily the first therapy given for a disease, designed/intended to bring about remission of the disease. Non-limiting examples of induction therapies, e.g., for AML, include daunorubicin-cytarabine (e.g., VYXEOS®); alvocidib-cytarabine-mitoxantrone (e.g., a combination therapy described herein comprising alvocidib, or a pharmaceutically acceptable salt thereof, cytarabine, or a pharmaceutically acceptable salt thereof, and mitoxantrone, or a pharmaceutically acceptable salt thereof); alvocidib-cytarabine-daunorubicin (e.g., a combination therapy described herein comprising alvocidib, or a pharmaceutically acceptable salt thereof, cytarabine, or a pharmaceutically acceptable salt thereof, and daunorubicin, or a pharmaceutically acceptable salt thereof); and alvocidib-cytarabine-idarubicin (e.g., a combination therapy described herein comprising alvocidib, or a pharmaceutically acceptable salt thereof, cytarabine, or a pharmaceutically acceptable salt thereof, and idarubicin, or a pharmaceutically acceptable salt thereof). Non-limiting examples of induction therapies, e.g., for MM, include bortezomib-lenalidomide-dexamethasone; lenalidomide and low-dose dexamethasone; bortezomib-cyclophosphamide-dexamethasone; daratumumab-bortezomib-melphalan-prednisone; carfilzomib-lenalidomide-dexamethasone; carfilzomib-cyclophosphamide-dexamethasone; ixazomib-lenalidomide-dexamethasone; bortezomib-dexamethasone; and cyclophosphamide-lenalidomide-dexamethasone.

In some embodiments, a prior therapy and/or induction therapy does not include alvocidib, or a prodrug thereof, or a pharmaceutically acceptable salt of the foregoing.

In some embodiments, a prior therapy and/or induction therapy (e.g., induction therapy) includes alvocidib, or a prodrug thereof, or a pharmaceutically acceptable salt of the foregoing. When a prior therapy and/or induction therapy includes alvocidib, or a prodrug thereof, or a pharmaceutically acceptable salt of the foregoing, and alvocidib, or a prodrug thereof, or a pharmaceutically acceptable salt of the foregoing, is administered in accordance with a maintenance therapy context described herein, the prior therapy and/or induction therapy will typically include alvocidib, or a pharmaceutically acceptable salt thereof, as, for example, when the prior therapy and/or induction therapy involves alvocidib-cytarabine-mitoxantrone (e.g., a combination therapy described herein comprising alvocidib, or a pharmaceutically acceptable salt thereof, cytarabine, or a pharmaceutically acceptable salt thereof, and mitoxantrone, or a pharmaceutically acceptable salt thereof), alvocidib-cytarabine-daunorubicin (e.g., a combination therapy described herein comprising alvocidib, or a pharmaceutically acceptable salt thereof, cytarabine, or a pharmaceutically acceptable salt thereof, and daunorubicin, or a pharmaceutically acceptable salt thereof) or alvocidib-cytarabine-idarubicin (e.g., a combination therapy described herein comprising alvocidib, or a pharmaceutically acceptable salt thereof, cytarabine, or a pharmaceutically acceptable salt thereof, and idarubicin, or a pharmaceutically acceptable salt thereof). In these embodiments, a prodrug of alvocidib, or a pharmaceutically acceptable salt thereof, will typically be administered in accordance with the described maintenance therapy context, typically, as an independent therapy (e.g., monotherapy, combination therapy) following completion of a prior and/or induction therapy, upon achievement of complete remission in the subject to maintain the complete remission.

Thus, some embodiments comprise administering to the subject an effective amount of a prodrug of alvocidib, or a pharmaceutically acceptable salt thereof, wherein the subject is in complete remission from the cancer (e.g., hematologic cancer) and MRD-positive following administration of a prior therapy that includes alvocidib, or a pharmaceutically acceptable salt thereof. Some embodiments comprise administering to the subject an effective amount of a prodrug of alvocidib, or a pharmaceutically acceptable salt thereof, wherein the subject is in complete remission from the cancer (e.g., hematologic cancer) and MRD-positive following administration of a prior therapy that includes alvocidib, or a pharmaceutically acceptable salt thereof; and is MRD-negative following administration of the prodrug of alvocidib, or a pharmaceutically acceptable salt thereof. Some embodiments comprise administering to the subject a maintenance therapy comprising an effective amount of a prodrug of alvocidib, or a pharmaceutically acceptable salt thereof, wherein the subject is in complete remission from the cancer (e.g., hematologic cancer) following administration of an induction therapy that includes alvocidib, or a pharmaceutically acceptable salt thereof. In some embodiments, the subject is MRD-positive following the induction therapy. In some embodiments, the subject is MRD-negative following administration of the prodrug of alvocidib, or a pharmaceutically acceptable salt thereof.

In some embodiments, a therapy (e.g., prior therapy, induction therapy) is a first-line therapy. As used herein, “first-line therapy” refers to the first therapy given for a disease or condition, wherein therapy is as used herein.

In some embodiments, a therapy (e.g., prior therapy, induction therapy) is a subsequent therapy. As used herein, “subsequent therapy” refers to any therapy given after a first-line therapy for a disease or condition, wherein therapy is as used herein. A subsequent therapy comprises one or more drugs that are different from the drug(s) of a first-line therapy. In some embodiments, the subsequent therapy is a second-line therapy (i.e., the second therapy given for a disease or condition). In some embodiments, the subsequent therapy is a third-line therapy (i.e., the third therapy given for a disease or condition).

Examples of therapeutic agents useful in therapies (e.g., prior therapies, induction therapies) described herein include, but are not limited to, bortezomib, lenalidomide, dexamethasone, cyclophosphamide, daratumumbab, melphalan, predisone, carfilzomib, ixazomib, cytarabine, daunorubicin, decitabine, azacytidine, hypomethylating agents (HMA), carmustine, doxorubicin, elotuzumab, panobinoastat, plerixafor, pomoalidone, thalidomide, zoledronic acid ibrutinib, duvelisib, idelalisib, obinutuzuma, and rituximab, or a pharmaceutically acceptable salt of any of the foregoing, or a combination of two or more of the foregoing.

Bortezomib, lenalidomide, dexamethasone, cyclophosphamide, daratumumbab, melphalan, predisone, carfilzomib, ixazomib and thalidomide, or a pharmaceutically acceptable salt of the foregoing, or a combination of two or more of the foregoing, are thought to be especially useful in methods wherein the hematologic cancer is multiple myeloma. For example, lenalidomide in combination with dexamethasone is indicated for the treatment of patients with MM who have received at least one prior therapy. The recommended starting dose of lenalidomide is 25 mg once daily, orally, on days 1-21 of repeated 28-day cycles. The recommended dose of dexamethasone is 40 mg once daily, orally, on days 1-4, 9-12 and 17-20 of each 28-day cycle for the first four cycles of therapy, and then 40 mg once daily, orally, on days 1˜4 every 28 days. Other combination therapies for MM include bortezomib, lenalidomide and dexamethasone; bortezomib, cyclophosphamide and dexamethasone; daratumumab, bortezomib, melphalan and prednisone; carfilzomib, lenalidomide and dexamethasone; carfilzomib, cyclophosphamide and dexamethasone; ixazomib, lenalidomide and dexamethasone; bortezomib and dexamethasone; and cyclophosphamide, lenalidomide and dexamethasone. Monotherapies for MM include lenalidomide and bortezomib.

In some embodiments, alvocidib, or a prodrug thereof, or a pharmaceutically acceptable salt of the foregoing, is administered in the absence of an additional chemotherapeutic agent, as a monotherapy.

“In the absence of an additional chemotherapeutic agent,” as used herein, means that the therapy does not involve combination therapy of alvocidib and an additional chemotherapeutic agent (e.g., azacitidine, decitabine). A subject (e.g., AML patient) of the therapies disclosed herein may be free of treatment with an additional chemotherapeutic agent after prior therapy(ies) involving the additional chemotherapeutic agent (either taken alone or in combination with other chemotherapies) terminates before commencement of the therapy excluding additional chemotherapeutic agents. A subject (e.g., AML patient) of the therapies disclosed herein may also or alternatively be free of treatment with an additional chemotherapeutic agent after prior therapy(ies) involving the additional chemotherapeutic agent (either taken alone or in combination with other chemotherapies) after a suitable washout period for the additional chemotherapeutic agent before commencement of the therapies disclosed herein. It will be understood that a subject (e.g., AML patient) of a monotherapy disclosed herein may be free of treatment with an additional chemotherapeutic agent but still be receiving non-chemotherapeutic agents, e.g., receiving supportive care.

In some embodiments, alvocidib, or a prodrug thereof, or a pharmaceutically acceptable salt of the foregoing, is administered as part of a combination therapy. In some embodiments, alvocidib, or a pharmaceutically acceptable salt of the foregoing, is administered with one or more additional chemotherapeutic agents.

The term “combination therapy” refers to a therapy, as that term is used herein, comprising administration of two or more therapeutic agents to treat a disease, disorder or condition described herein. Such administration encompasses co-administration of the therapeutic agents in a substantially simultaneous manner, such as in a pharmaceutical combination. Alternatively, such administration encompasses co-administration in multiple containers, or separate containers (e.g., capsules, powders, and liquids) for each active ingredient, such as in a kit. Such administration also encompasses use of each type of therapeutic agent in a sequential manner, either at approximately the same time or at different times. A therapeutic agent and an additional therapeutic agent(s) can be administered via the same administration route or via different administration routes.

In combination therapies, the therapeutic agents may be manufactured and/or formulated by the same or different manufacturers. Moreover, the therapeutic agents may be brought together into a combination therapy, e.g.: (i) prior to release of the combination product to physicians (e.g., in the case of a kit or pharmaceutical combination comprising the therapeutic agents); (ii) by the physician (or under the guidance of a physician) shortly before administration; (iii) in the subjects, e.g., during sequential administration of the therapeutic agents.

Thus, in some embodiments, alvocidib, or a prodrug thereof, or a pharmaceutically acceptable salt of the foregoing is administered in combination with a hypomethylating agent (e.g., a therapeutically effective amount of a hypomethylating agent). Hypomethylating agents inhibit DNA methylation (e.g., by inhibiting the activity of a DNA methyltransferase), and can induce re-expression of the NOXA gene, which is a natural inhibitor of MCL-1. In keeping with this mechanism of action, the HMA azacitidine has been shown to reduce MCL-1 protein levels in blasts. See Konopleva and Letai, Blood 132(10):1007-1012. Non-limiting examples of hypomethylating agents include azacitidine (e.g., CC-486), or a prodrug thereof (such as a phosphate prodrug or 2′,3′,5′-triacetyl-5-azacitidine), and decitabine (e.g., ASTX727), or a prodrug thereof. Phosphate prodrugs of azacitidine suitable for use in the present methods are disclosed in International Publication No. WO 2011/153374, which is hereby incorporated by reference in its entirety. In some embodiments, the phosphate prodrug of azacitidine has the formula:

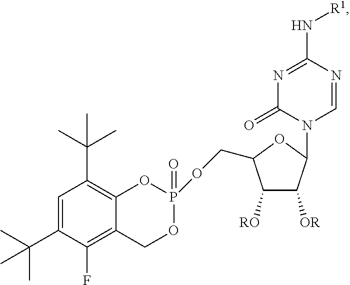

or a pharmaceutically acceptable salt or solvate thereof, wherein R and R¹ are independently H or CO₂(C₁-C₆ alkyl) (e.g., each R is H and R¹ is CO₂(C₅-alkyl)). Prodrugs of azacitidine, including phosphate prodrugs and 2′,3′,5′-triacetyl-5-azacitidine, can be administered orally. Decitabine, for example, is indicated for MDS, including previously treated and untreated, de novo and secondary MDS of all French-American-British subtypes (refractory anemia, refractory anemia with ringed sideroblasts, refractory anemia with excess blasts, refractory anemia with excess blasts in transformation, and chronic myelomonocytic leukemia) and intermediate-1, intermediate-2, and high-risk International Prognostic Scoring System. The recommended dose of decitabine is 15 mg/m² by continuous intravenous infusion over three hours, repeated every eight hours for three days, on a six-week cycle. Decitabine can also be administered at a dose of 20 mg/m² by continuous intravenous infusion over one hour, repeated daily for five days, on a four-week cycle.

ASTX727 is in Phase 3 clinical trials for subjects with MDS, chronic myelomonocytic leukemia and AML, and is a tablet for oral administration containing a fixed-dose combination of 100 mg cedazuridine (a cytidine deaminase inhibitor) and 35 mg decitabine, given by mouth daily for five consecutive days (e.g., days 1-5) in 28-day cycles (e.g., in Cycle 1 or Cycle 2, then in Cycle 3 and beyond). Subjects receiving ASTX727 are required to fast from food for 4 hours on days when receiving ASTX727: at least 2 hours before and 2 hours after dosing.

In combination therapies involving alvocidib, or a prodrug thereof, or a pharmaceutically acceptable salt thereof (e.g., alvocidib, or a pharmaceutically acceptable salt thereof), from about 15 mg/m² to about 50 mg/m², preferably, about 20 mg/m², decitabine can be administered to a subject once per day by intravenous infusion of about 1 hour in duration for from three to ten (e.g., consecutive) days. In some embodiments, from about 15 mg/m² to about 50 mg/m², preferably, about 35 mg/m², decitabine can be administered to a subject once per day orally for from three to ten (e.g., consecutive) days (e.g., 5 days, such as 5 consecutive days). In some embodiments, about 35 mg/m² decitabine and about 100 mg/m² cedazuridine (e.g., ASTX727) can be administered to a subject once per day orally for from three to ten (e.g., consecutive) days (e.g., 5 days, such as 5 consecutive days). In some embodiments, an effective amount or therapeutically effective amount of ASTX727 can be administered to a subject, e.g., once per day orally for from three to ten (e.g., consecutive) days (e.g., 5 days, such as 5 consecutive days). In some embodiments, from about 15 mg/m² to about 50 mg/m² decitabine can be administered to a subject once per day for from three to ten (e.g., consecutive) days (e.g., 5 days, such as 5 consecutive days). In some embodiments, decitabine is administered daily for 3 (e.g., consecutive) days. In some embodiments, decitabine is administered daily for 5 days (e.g., on days 1-5 of a treatment schedule or cycle). In some embodiments, decitabine is administered daily for 10 (e.g., consecutive) days. When alvocidib, or a pharmaceutically acceptable salt thereof, is used in combination with a decitabine, the alvocidib, or a pharmaceutically acceptable salt thereof, can be administered once on day 8 of the treatment schedule using any of the dosages and dosing schedules for alvocidib, or a pharmaceutically acceptable salt described herein (e.g., by intravenous bolus of about 30 minutes in duration in a dose of about 30 mg/m² alvocidib, or a pharmaceutically acceptable salt thereof, followed by an intravenous infusion of about 4 hours in duration in a dose of about 60 mg/m² alvocidib, or a pharmaceutically acceptable salt thereof; by intravenous infusion of about one hour in duration in a dose of about 90 mg/m² alvocidib, or a pharmaceutically acceptable salt thereof). When this treatment schedule is employed, days 6 and 7 are typically drug holidays.

Azacitidine is indicated for patients with the following FAB myelodysplastic syndrome (MDS) subtypes: Refractory anemia (RA) or refractory anemia with ringed sideroblasts (RARS) (if accompanied by neutropenia or thrombocytopenia or requiring transfusions), refractory anemia with excess blasts (RAEB), refractory anemia with excess blasts in transformation (RAEB-T), and chronic myelomonocytic leukemia (CMMoL). The recommended starting dose of azacitidine for the first treatment cycle, for all patients regardless of baseline hematology values, is 75 mg/m² daily for 7 days, to be administered by subcutaneous injection or intravenous infusion, on a four-week cycle for a minimum of 4 to 6 cycles. After 2 cycles, the dose of azacitidine may be increased to 100 mg/m².

CC-486 is in clinical trials for subject with AML and MDS, and is an oral formulation of azacitidine.

In combination therapies involving alvocidib, or a prodrug thereof, or a pharmaceutically acceptable salt thereof (e.g., alvocidib, or a pharmaceutically acceptable salt thereof), from about 50 mg/m² to about 125 mg/m², preferably, about 75 mg/m², azacitidine, or a prodrug thereof, or a pharmaceutically acceptable salt of the foregoing (e.g., azacitidine, or a pharmaceutically acceptable salt thereof), can be administered to a subject once per day by intravenous infusion of about 10 minutes to about 40 minutes in duration or by subcutaneous injection for from five to ten days, preferably for 5 days or 7 days. In some embodiments, azacitidine, or a prodrug thereof, or a pharmaceutically acceptable salt of the foregoing (e.g., azacitidine, or a pharmaceutically acceptable salt thereof), is administered daily for 7 consecutive days (e.g., on days 1-7 of a treatment schedule or cycle).

In combination therapies involving alvocidib, or a prodrug thereof, or a pharmaceutically acceptable salt thereof (e.g., a prodrug of alvocidib, or a pharmaceutically acceptable salt thereof), azacitidine, or a prodrug thereof, or a pharmaceutically acceptable salt of the foregoing (e.g., azacitidine, or a pharmaceutically acceptable salt thereof), from about 150 mg/m² to about 300 mg/m², preferably, about 200 mg/m², azacitidine, or a pharmaceutically acceptable salt thereof (e.g., CC-486), is administered to a subject once per day orally, e.g., for 7, 14 or 21 days (e.g., on days 1-7, 1-14 or 1-21, respectively, of a 21-day or 28-day cycle). In some embodiments, an effective amount or a therapeutically effective amount of CC-486 is administered to a subject once per day orally, e.g., for 7, 14 or 21 days (e.g., on days 1-7, 1-14 or 1-21, respectively, of a 21-day or 28-day cycle).

When alvocidib, or a pharmaceutically acceptable salt thereof, is used in combination with azacitidine, the alvocidib, or a pharmaceutically acceptable salt thereof, can be administered once on day 10 of the treatment schedule or cycle using any of the dosages and dosing schedules for alvocidib, or a pharmaceutically acceptable salt thereof, described herein (e.g., by intravenous infusion of about one hour in duration in a dose of about 90 mg/m² alvocidib, or a pharmaceutically acceptable salt thereof). When this treatment schedule or cycle is employed, days 8 and 9 are typically drug holidays.

Alternatively, azacitidine, or a pharmaceutically acceptable salt thereof, can be administered according to a 5-2-2 regimen, in which azacitidine, or a pharmaceutically acceptable salt thereof, is administered once daily for 5 days (e.g., on days 1-5 of a treatment schedule or cycle) and once daily for 2 days (e.g., on days 8 and 9 of the treatment schedule or cycle). When alvocidib, or a pharmaceutically acceptable salt thereof, is used in combination with azacitidine, or a pharmaceutically acceptable salt thereof, according to a 5-2-2 regimen, the alvocidib, or a pharmaceutically acceptable salt thereof, can be administered once on day 10 of the treatment schedule using any of the dosages and dosing schedules for alvocidib, or a pharmaceutically acceptable salt thereof, described herein (e.g., by intravenous infusion of about one hour in duration in a dose of about 90 mg/m² alvocidib, or a pharmaceutically acceptable salt thereof). When this treatment schedule or cycle is employed, days 6 and 7 are typically drug holidays.

Combination therapy comprising alvocidib, or a prodrug thereof, or a pharmaceutically acceptable salt of the foregoing, and a hypomethylating agent (e.g., azacitidine, or a prodrug thereof, or decitabine, or a prodrug thereof) are thought to be particularly useful in methods wherein the cancer is MDS or AML.

In some embodiments, alvocidib, or a prodrug thereof, or a pharmaceutically acceptable salt of the foregoing (e.g., alvocidib, or a pharmaceutically acceptable salt thereof), is administered in combination with cytarabine (e.g., a therapeutically effective amount of cytarabine), or a pharmaceutically acceptable salt thereof, e.g., in the absence of an additional chemotherapeutic agent. Combination therapies involving alvocidib, or a prodrug thereof, or a pharmaceutically acceptable salt of the foregoing, and cytarabine, or a pharmaceutically acceptable salt thereof, are thought to be particularly useful for the treatment of AML, such as relapsed or refractory AML or AML resistant to venetoclax or venetoclax in combination with a hypomethylating agent.

In some embodiments, a subject may be resistant to venetoclax or venetoclax and a HMA, or be refractory and/or have relapsed after one or more treatments including venetoclax or venetoclax and a HMA.

As used herein, the term “resistant” has its ordinary meaning in the art, and may refer to a cancer, such as AML, that does not respond to treatment. A cancer may be resistant at the beginning of treatment or it may become resistant during treatment. For instance, a cancer, such as AML, may become resistant after one or more treatments (e.g., including up to two treatment cycles comprising a BCL-2 inhibitor) or after one or more treatment cycles.

As used herein, the term “refractory” has its ordinary meaning in the art, and may refer to a subject that has residual leukemic cells in its (e.g., his/her) marrow after treatment. In some embodiments, “refractory” means a subject failed to achieve CR (e.g., CR wherein less than 5% of the cells in the bone marrow are blasts, and there is an absence of blasts with Auer rods in the bone marrow, an absence of extramedullary disease, and full hematologic recovery (e.g., absolute neutrophil count (ANC) ≥1,000/μL and platelet count ≥100,000/μL), and/or CRᵢ) following treatment for a disease, or achieved a CR (e.g., CR wherein less than 5% of the cells in the bone marrow are blasts, and there is an absence of blasts with Auer rods in the bone marrow, an absence of extramedullary disease, and full hematologic recovery (e.g., absolute neutrophil count (ANC) ≥1,000/μL and platelet count ≥100,000/μL), and/or CRᵢ) lasting less than 90 days following treatment for the disease.

As used herein, the term “relapse” or “relapsed” has its ordinary meaning in the art, and may refer to the return of a cancer, such as AML, or the signs and symptoms of a cancer, such as AML, after a period of complete remission (e.g., initial complete remission) due to treatment. In some embodiments, relapse may refer to the recurrence of disease after complete remission meeting one or more of the following criteria (i) >5% blasts in the marrow or peripheral blood, and/or (ii) extramedullary disease, and/or disease presence determined by a physician upon clinical assessment. In some embodiments, “relapse” refers to reoccurrence of a disease following a CR (e.g., CR wherein less than 5% of the cells in the bone marrow are blasts, and there is an absence of blasts with Auer rods in the bone marrow, an absence of extramedullary disease, and full hematologic recovery (e.g., absolute neutrophil count (ANC) ≥1,000/μL and platelet count ≥100,000 μL), and/or CRᵢ) lasting 90 days or longer.

In some embodiments involving combination therapy comprising alvocidib, or a prodrug thereof, or a pharmaceutically acceptable salt of the foregoing, and cytarabine, or a pharmaceutically acceptable salt thereof, alvocidib, or a prodrug thereof, or a pharmaceutically acceptable salt of the foregoing, is administered on days 1 and 15 of a 28-day treatment cycle, and cytarabine, or a pharmaceutically acceptable salt thereof, is administered for ten consecutive days (e.g., on days 3-12) of the 28-day treatment cycle. For example, from about 15 mg/m² to about 40 mg/m² (e.g., about 25 mg/m²) alvocidib, or a pharmaceutically acceptable salt thereof, can be administered by intravenous bolus on day 1 of a 28-day treatment cycle; from about 40 mg/m² to about 80 mg/m² (e.g., about 50 mg/m²) alvocidib, or a pharmaceutically acceptable salt thereof, can be administered by intravenous bolus on day 15 of the 28-day treatment cycle; and from about 10 mg/m² to about 100 mg/m² (e.g., about 20 mg/m²) cytarabine, or a pharmaceutically acceptable salt thereof, can be administered per day by injection on days 3, 4, 5, 6, 7, 8, 9, 10, 11 and 12 of the 28-day treatment cycle.

In some embodiments, alvocidib, or a prodrug thereof, or a pharmaceutically acceptable salt of the foregoing, is administered in combination with cytarabine (e.g., a therapeutically effective amount of cytarabine), or a pharmaceutically acceptable salt thereof, and daunorubicin or idarubicin (e.g., a therapeutically effective amount of daunorubicin or idarubicin), or a pharmaceutically acceptable salt of the foregoing, e.g., for the treatment of AML. In some embodiments, alvocidib, or a prodrug thereof, or a pharmaceutically acceptable salt of the foregoing, is administered in combination with cytarabine (e.g., a therapeutically effective amount of cytarabine), or a pharmaceutically acceptable salt thereof, and daunorubicin (e.g., a therapeutically effective amount of daunorubicin), or a pharmaceutically acceptable salt thereof, e.g., for the treatment of AML.

Alvocidib, or a prodrug thereof, or a pharmaceutically acceptable salt of the foregoing (e.g., alvocidib, or a pharmaceutically acceptable salt of the foregoing) can be added to cytarabine and daunorubicin or idarubicin, administered according to a so-called 7+3 regimen. In a 7+3 regimen, and daunorubicin or idarubicin, or a pharmaceutically acceptable salt of the foregoing, can be administered daily for three days of a treatment schedule (e.g., days 5-7). In a 7+3 regimen, cytarabine, or a pharmaceutically acceptable salt thereof, can be administered daily for seven days of a treatment schedule (e.g., days 5-11) by intravenous infusion of from about 20 hours to about 28 hours (e.g., about 24 hours) in duration in a dose of from about 90 mg/m² to about 110 mg/m² (e.g., about 100 mg/m²). Daunorubicin or idarubicin (e.g., daunorubicin), or a pharmaceutically acceptable salt of the foregoing, can be administered daily for three days of the treatment schedule (e.g., days 5-7) by intravenous bolus of from about 5 minutes to about 30 minutes (e.g., about 15 minutes) in duration in a dose of from about 45 mg/m² to about 110 mg/m² (e.g., about 60 mg/m²). When alvocidib, or a pharmaceutically acceptable salt thereof, is used in combination with a 7+3 regimen, the alvocidib, or a pharmaceutically acceptable salt thereof, can be administered daily on days 1-3 of the treatment schedule, e.g., using a hybrid dose of alvocidib, or a pharmaceutically acceptable salt thereof (e.g., by intravenous bolus of about 30 minutes in duration in a dose of about 30 mg/m² alvocidib, or a pharmaceutically acceptable salt thereof, followed by an intravenous infusion of about 4 hours in duration in a dose of about 60 mg/m² alvocidib, or a pharmaceutically acceptable salt thereof).

Accordingly, in some embodiments involving combination therapies comprising alvocidib, or a prodrug thereof, or a pharmaceutically acceptable salt of the foregoing, cytarabine, or a pharmaceutically acceptable salt thereof, and daunorubicin or idarubicin, or a pharmaceutically acceptable salt of the foregoing, alvocidib, or a prodrug thereof, or a pharmaceutically acceptable salt of the foregoing, is administered to the subject on the first, second and third days of a treatment; daunorubicin or idarubicin (e.g., daunorubicin), or a pharmaceutically acceptable salt of the foregoing, is administered to the subject on the fifth, sixth and seventh days of the treatment; and cytarabine, or a pharmaceutically acceptable salt thereof, is administered to the subject on the fifth, sixth, seventh, eighth, ninth, tenth, and eleventh days of the treatment. In some embodiments, the method comprises administering:

from about 5 mg/m² to about 50 mg/m² (e.g., about 30 mg/m²) alvocidib, or a pharmaceutically acceptable salt thereof, per day, administered by an intravenous bolus of from about 15 minutes to about 45 minutes (e.g., about 30 minutes) in duration, and from about 10 mg/m² to about 65 mg/m² (e.g., about 60 mg/m²) alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;

from about 45 mg/m² to about 110 mg/m² (e.g., about 60 mg/m²) daunorubicin, or a pharmaceutically acceptable salt thereof, per day, administered by intravenous bolus of from about 5 minutes to about 30 minutes in duration on the fifth, sixth and seventh days of the treatment; and

from about 90 mg/m² to about 110 mg/m² (e.g., about 100 mg/m²) cytarabine, or a pharmaceutically acceptable salt thereof, per day, administered by intravenous infusion of from about 20 hours to about 28 hours (e.g., about 24 hours) in duration on the fifth, sixth, seventh, eighth, ninth, tenth, and eleventh days of the treatment.

If such treatment does not result in complete remission, the 7+3 regimen can be followed, e.g., beginning on day 15 (such that day 1 of the 5+2 regimen corresponds to day 15 of the 7+3 regimen), by a 5+2 regimen, administered in combination with alvocidib, or a prodrug thereof, or a pharmaceutically acceptable salt of the foregoing (e.g., alvocidib, or a pharmaceutically acceptable salt thereof). A “5+2 regimen” is a regimen in which cytarabine, or a pharmaceutically acceptable salt thereof, is administered daily for five days (e.g., days 5-9) and daunorubicin or idarubicin, or a pharmaceutically acceptable salt of the foregoing, is administered daily for two days (e.g., days 5-6). In a 5+2 regimen, cytarabine, or a pharmaceutically acceptable salt thereof, can be administered daily for five days of a treatment schedule (e.g., days 5-9) by intravenous infusion of from about 20 hours to about 28 hours (e.g., about 24 hours) in duration in a dose of from about 90 mg/m² to about 110 mg/m² (e.g., about 100 mg/m²). Daunorubicin or idarubicin (e.g., daunorubicin), or a pharmaceutically acceptable salt of the foregoing, can be administered daily for two days of the treatment schedule (e.g., days 5-6) by intravenous bolus of from about 5 minutes to about 30 minutes (e.g., about 15 minutes) in duration in a dose of from about 30 mg/m² to about 60 mg/m² (e.g., about 45 mg/m²). When alvocidib, or a pharmaceutically acceptable salt thereof, is used in combination with a 5+2 regimen, the alvocidib, or a pharmaceutically acceptable salt thereof, can be administered daily on days 1-3 of the treatment schedule, e.g., using a hybrid dose of alvocidib, or a pharmaceutically acceptable salt thereof (e.g., by intravenous bolus of about 30 minutes in duration in a dose of about 30 mg/m² alvocidib, or a pharmaceutically acceptable salt thereof, followed by an intravenous infusion of about 4 hours in duration in a dose of about 60 mg/m² alvocidib, or a pharmaceutically acceptable salt thereof).

Thus, in some embodiments, the method comprises administering to the subject a first treatment comprising:
from about 5 mg/m² to about 50 mg/m² (e.g., about 30 mg/m²) alvocidib, or a pharmaceutically acceptable salt thereof, per day, administered by an intravenous bolus of from about 15 minutes to about 45 (e.g., about 30) minutes in duration, and from about 10 mg/m² to about 65 mg/m² (e.g., about 60 mg/m²) alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the first treatment, and the intravenous infusion is initiated about 30 minutes after completion of the intravenous bolus;
from about 45 mg/m² to about 110 mg/m² (e.g., about 60 mg/m²) daunorubicin, or a pharmaceutically acceptable salt thereof, per day, administered by intravenous bolus of from about 5 minutes to about 30 minutes in duration on the fifth, sixth and seventh days of the first treatment; and
from about 90 mg/m² to about 110 mg/m² (e.g., about 100 mg/m²) cytarabine, or a pharmaceutically acceptable salt thereof, per day, administered by intravenous infusion of from about 20 hours to about 28 hours in duration on the fifth, sixth, seventh, eighth, ninth, tenth, and eleventh days of the first treatment; and

a second treatment comprising:
from about 5 mg/m² to about 50 mg/m² (e.g., about 30 mg/m²) alvocidib, or a pharmaceutically acceptable salt thereof, per day, administered by an intravenous bolus of from about 15 minutes to about 45 (e.g., about 30) minutes in duration, and from about 10 mg/m² to about 65 mg/m² (e.g., about 60 mg/m²) alvocidib, or a pharmaceutically acceptable salt thereof, per day, administered by intravenous infusion of about 4 hours in duration, wherein the intravenous bolus and the intravenous infusion of alvocidib, or a pharmaceutically acceptable salt thereof, are administered to the subject on the first, second and third days of the second treatment, and the intravenous infusion is initiated about 30 minutes after completion of the intravenous bolus;
from about 30 mg/m² to about 60 mg/m² (e.g., about 45 mg/m²) daunorubicin, or a pharmaceutically acceptable salt thereof, per day, administered by intravenous bolus of from about 5 minutes to about 30 minutes in duration on the fifth and sixth days of the second treatment; and
from about 90 mg/m² to about 110 mg/m² (e.g., about 100 mg/m²) cytarabine, or a pharmaceutically acceptable salt thereof, per day, administered by intravenous infusion of from about 20 hours to about 28 hours in duration on the fifth, sixth, seventh, eighth and ninth days of the second treatment,
wherein the first day of the second treatment corresponds to the fifteenth day of the first treatment.

In some embodiments, alvocidib, or a prodrug thereof, or a pharmaceutically acceptable salt of the foregoing, is administered in combination with cytarabine (e.g., a therapeutically effective amount of cytarabine), or a pharmaceutically acceptable salt thereof, and mitoxantrone (e.g., a therapeutically effective amount of mitoxantrone), or a pharmaceutically acceptable salt thereof (e.g., mitoxantrone hydrochloride), e.g., for the treatment of AML.

In combination therapies involving alvocidib, or a prodrug thereof, or a pharmaceutically acceptable salt of the foregoing, cytarabine, or a pharmaceutically acceptable salt thereof, and mitoxantrone, or a pharmaceutically acceptable salt thereof, alvocidib can be administered once daily for three consecutive days (e.g., on days 1-3 of a treatment schedule or cycle), cytarabine, or a pharmaceutically acceptable salt thereof, can be administered for three consecutive days (e.g., on days 6-8 of the treatment schedule or cycle, such as by continuous IV infusion of a duration of about 72 hours) and mitoxantrone, or a pharmaceutically acceptable salt thereof, can be administered once (e.g., on day 9 of the treatment schedule or cycle, such as by intravenous infusion of from about 1 to about 2 hours in duration, beginning about twelve hours after completion of the cytarabine treatment). For example, about 667 mg/m² cytarabine, or a pharmaceutically acceptable salt thereof, can be administered per day, such that about 2 gm/m² cytarabine is administered over the course of an infusion having a duration of about 72 hours. About 40 mg/m² mitoxantrone, or a pharmaceutically acceptable salt thereof, can be administered per day. The alvocidib, or a pharmaceutically acceptable salt thereof, can be administered, e.g., using a hybrid dose of alvocidib, or a pharmaceutically acceptable salt thereof (e.g., by intravenous bolus of about 30 minutes in duration in a dose of about 30 mg/m² alvocidib, or a pharmaceutically acceptable salt thereof, followed by an intravenous infusion of about 4 hours in duration in a dose of about 60 mg/m² alvocidib, or a pharmaceutically acceptable salt thereof). In the nine-day regimen described above, days 4 and 5 are typically drug holidays.

In a specific embodiment, a method of treating a hematologic cancer (e.g., MM, such as relapsed and/or refractory multiple myeloma) in a subject in need thereof (e.g., a transplant-ineligible subject having MM) is provided. The method comprises administering to the subject an effective amount of alvocidib, or a prodrug thereof, or a pharmaceutically acceptable salt of the foregoing (e.g., a compound of Structural Formula Ia, or a pharmaceutically acceptable salt thereof), wherein the subject is in complete remission from the MM and MRD-positive following a prior maintenance therapy that includes lenalidomide, or a pharmaceutically acceptable salt thereof, and does not include alvocidib, or a prodrug thereof, or a pharmaceutically acceptable salt of the foregoing.

Lenalidomide, or a pharmaceutically acceptable salt thereof, is thought to be useful in methods wherein the hematologic cancer is MDS. For example, lenalidomide monotherapy is indicated for the treatment of patients with transfusion-dependent anemia due to low- or intermediate-1-risk myelodysplastic syndromes (MDS) associated with a deletion 5q cytogenetic abnormality with or without additional cytogenetic abnormalities. The recommended starting dose of lenalidomide is 25 mg once daily, orally, on days 1-21 of repeated 28-day cycles.

Cytarabine and daunorubicin, or a pharmaceutically acceptable salt of the foregoing, or a combination of two or more of the foregoing, are thought to be especially useful in methods wherein the hematologic cancer is AML. For example, VYXEOS®, a liposomal combination of daunorubicin and cytarabine, is indicated for the treatment of adults with newly-diagnosed therapy-related AML, or AML with myelodysplasia-related changes. The recommended induction dose of VYXEOS® contains 44 mg/m² daunorubicin and 100 mg/m² cytarabine, and is administered via intravenous infusion over 90 minutes on days 1, 3 and 5 of a 2- to 5-week cycle, and days 1 and 3 for a subsequent cycle of induction, if needed. The recommended consolidation dose of VYXEOS® is 29 mg/m² daunorubicin and 65 mg/m² cytarabine, administered 5 to 8 weeks after the start of the last induction cycle via intravenous infusion over 90 minutes on days 1 and 3 of a 5- to 8-week cycle.

In a specific embodiment, a method of treating a hematologic cancer (e.g., AML, such as AML with myelodysplastic features) in a subject in need thereof is provided. The method comprises administering to the subject an effective amount of alvocidib, or a prodrug thereof, or a pharmaceutically acceptable salt of the foregoing (e.g., alvocidib, or a pharmaceutically acceptable salt thereof; a compound of Structural Formula Ia, or a pharmaceutically acceptable salt thereof), wherein the subject is in complete remission from the AML and MRD-positive following a prior induction therapy that includes daunorubicin, or a pharmaceutically acceptable salt thereof, and/or cytarabine, or a pharmaceutically acceptable salt thereof, and does not include alvocidib, or a prodrug thereof, or a pharmaceutically acceptable salt of the foregoing. In some embodiments, the prior therapy is a frontline therapy. In some embodiments, the subject is unfit. In some embodiments, the daunorubicin, or a pharmaceutically acceptable salt thereof, and/or cytarabine, or a pharmaceutically acceptable salt thereof, are/is provided in a liposomal composition (e.g., liposomal combination). In some embodiments, the prior induction therapy includes about 44 mg/m² daunorubicin and about 100 mg/m² cytarabine, administered via intravenous infusion over about 90 minutes on days 1, 3 and 5 of a cycle, or on days 1 and 3 of a cycle.

Many therapies are administered on a treatment cycle, or cycle. As used herein, “treatment cycle” and “cycle” are used interchangeably to refer to a therapy (e.g., schedule or course of therapy comprising periods of treatment and periods of no treatment) that is repeated on a regular or substantially regular schedule. The length of a treatment cycle is determined by the treatment being administered, but can be 1, 2, 3, 4, 5, 6, 7, 8, 9, 10, 11, 12, 13, 14, 15, 16, 17, 18, 19, 20, 21, 22, 23, 24, 25, 26, 27 or 28 days, or 4, 5, 6, 7, 8, 9, 10, 11 or 12 weeks.

In some embodiments, alvocidib, or a prodrug thereof, or a pharmaceutically acceptable salt of the foregoing, and/or a therapy (e.g., induction therapy, maintenance therapy, prior therapy) is administered on a cycle, for example, a 10-day, 11-day, 12-day, 13-day, 14-day, 15-day, 20-day, 21-day, 28-day or 42-day cycle (e.g., a 28-day cycle). For example, one or more cycles (e.g., 1, 2, 3, 4, 5, 6, 7, 8, 9, 10, 11, 12, 15, 18, 21, 24 etc. cycles) of alvocidib, or a prodrug thereof, or a pharmaceutically acceptable salt of the foregoing, and/or a therapy (e.g., induction therapy, maintenance therapy, prior therapy) can be administered to a subject in accordance with the methods disclosed herein. Typically, when a therapy (e.g., prior therapy, induction therapy) is administered on a cycle, day 1 of the therapy cycle corresponds to day 1 of the cycle (e.g., 28-day cycle) of alvocidib, or a prodrug thereof, or a pharmaceutically acceptable salt of the foregoing. In some embodiments, a treatment cycle is 21 days in duration. In some embodiments, a treatment cycle is 28 days in duration.

In some embodiments, alvocidib, or a prodrug thereof, or a pharmaceutically acceptable salt of the foregoing (e.g., a compound of structural formula Ia), is administered continuously. Alternatively, administration of alvocidib, or a prodrug thereof, or a pharmaceutically acceptable salt of the foregoing (e.g., a compound of structural formula Ia), may include treatment interruptions. For a dosing schedule including treatment interruptions, alvocidib, or a prodrug thereof, or a pharmaceutically acceptable salt of the foregoing (e.g., a compound of structural formula Ia), may be administered on a treatment cycle including a time period of continuous dosing, followed by a treatment interruption wherein the alvocidib, or a prodrug thereof, or a pharmaceutically acceptable salt of the foregoing, is not administered. The treatment interruption may be, for example, 1, 2, 3, 4, 5, 6, 7, 14, or 28 days. In certain particular embodiments, the dosing schedule is a 21-day treatment cycle including 14 days of dosing, followed by a treatment interruption of 7 days. In other particular embodiments, the dosing schedule is a 28-day treatment cycle including 21 days of dosing (e.g., BID dosing, QD dosing), followed by a treatment interruption of 7 days. Stated otherwise, in some embodiments, alvocidib, or a prodrug thereof, or a pharmaceutically acceptable salt of the foregoing (e.g., a compound of structural formula Ia), is administered on the first 21 days of a 28-day treatment cycle, and is not administered on days 22 to 28 of the 28-day treatment cycle.

In yet other particular embodiments, a treatment cycle and/or dosing schedule includes one or more (e.g., one, two, three) days on which alvocidib, or a prodrug thereof, or a pharmaceutically acceptable salt of the foregoing (e.g., alvocidib, or a pharmaceutically acceptable salt thereof), is administered, e.g., on days 1-3; days 1, 8 and 15; day 8; day 10; days 1 and 15.

In some embodiments, a subject is administered at least one cycle (e.g., one, two, three, four, five, six, seven, eight, nine, 10, 11, 12, 15, 18, 21, 24 cycles) of a therapy (e.g., prior therapy, induction therapy) prior to being administered alvocidib, or a prodrug thereof, or a pharmaceutically acceptable salt of the foregoing.

In some embodiments, a subject is administered a therapy (e.g., prior therapy, induction therapy) for at least or about one month, at least or about two months, at least or about three months, at least or about six months, at least or about nine months or at least or about one year prior to being administered alvocidib, or a prodrug thereof, or a pharmaceutically acceptable salt of the foregoing.

In some embodiments, a therapy (e.g., prior therapy, induction therapy) includes lenalidomide, or a pharmaceutically acceptable salt thereof. In some embodiments, a therapy (e.g., prior therapy, induction therapy) includes lenalidomide, or a pharmaceutically acceptable salt thereof, and dexamethasone, or a pharmaceutically acceptable salt thereof. When a therapy includes lenalidomide, or a pharmaceutically acceptable salt thereof, the therapy typically includes from about 2.5 mg to about 25 mg (e.g., about 25 mg) lenalidomide, or a pharmaceutically acceptable salt thereof, administered to a subject orally once daily on days 1-21 of a 28-day treatment cycle. When a therapy includes lenalidomide, or a pharmaceutically acceptable salt thereof, and dexamethasone, or a pharmaceutically acceptable salt thereof, the therapy can include from about 2.5 mg to about 25 mg (e.g., about 25 mg) lenalidomide, or a pharmaceutically acceptable salt thereof, administered to a subject orally once daily on days 1-21 of a 28-day treatment cycle; and about 40 mg of dexamethasone, or a pharmaceutically acceptable salt thereof, administered to a subject orally once daily on days 1-4, 9-12 and 17-20 of the 28-day treatment cycle, or on days 1˜4 of the 28-day treatment cycle. In some embodiments, the subject continues to receive lenalidomide, or a pharmaceutically acceptable salt thereof, for at least a portion of the time (e.g., the entire time) the subject is being administered alvocidib, or a prodrug thereof, or a pharmaceutically acceptable salt of the foregoing.

“Effective amount” refers to an amount of a therapeutic agent which, when administered to a subject, such as a human, is at least sufficient to maintain the subject in complete remission from a cancer (e.g., hematologic cancer, such as MM) from which the subject suffers(ed). The amount of a therapeutic agent that constitutes an “effective amount” will vary, for example, depending on the therapeutic agent or combination of therapeutic agents, the condition being treated and its severity, the manner of administration, the duration of treatment, and/or the subject to be treated (e.g., the age, weight, fitness, etc. of the subject to be treated), but can be determined routinely by one of ordinary skill in the art based on his own knowledge and this disclosure. In embodiments, an “effective amount” is measured by a statistically significant and beneficial change in one or more indications, symptoms, signs, diagnostic tests, vital signs, and the like. In other embodiments, an “effective amount” manages or prevents a condition, as measured by a lack of a statistically significant change in one or more indications, symptoms, signs, diagnostic tests, vital signs, and the like.

“Therapeutically effective amount” refers to an amount of a therapeutic agent that, when administered to a subject, such as a human, is sufficient to effect treatment. The amount of a therapeutic agent that constitutes a “therapeutically effective amount” will vary, for example, depending on the therapeutic agent or combination of therapeutic agents, the condition being treated and its severity, the manner of administration, the duration of treatment, and/or the subject to be treated (e.g., the age, weight, fitness, etc. of the subject to be treated), but can be determined routinely by one of ordinary skill in the art based on his own knowledge and this disclosure. In embodiments, a “therapeutically effective amount” is measured by a statistically significant change in one or more indications, symptoms, signs, diagnostic tests, vital signs, and the like (e.g., by a determination of partial or complete remission). In other embodiments, a “therapeutically effective amount” manages or prevents a condition as measured by a lack of a statistically significant change in one or more indications, symptoms, signs, diagnostic tests, vital signs, and the like. In some embodiments, a therapeutically effective amount refers to an amount of a therapeutic agent which, when administered to a subject, such as a human, is at least sufficient to bring about complete remission in a subject suffering from a cancer (e.g., hematologic cancer, such as AML).

As used herein, “statistically significant” refers to a p value of 0.050 or less when calculated using the Students t-test, and indicates that it is unlikely that a particular event or result being measured has arisen by chance.

In some embodiments, an effective and/or therapeutically effective amount of alvocidib, or a pharmaceutically acceptable salt thereof, is administered to a subject, for example, once weekly for three or four (e.g., three) consecutive weeks, for example, on a 28-day or 42-day (e.g., 28-day) cycle. In some embodiments, an effective and/or therapeutically effective amount of alvocidib, or a pharmaceutically acceptable salt thereof, is administered to a subject daily for three consecutive days, for example, on a 28-day cycle.

Alvocidib (also known as flavopiridol) is 2-(2-chlorophenyl)-5,7-dihydroxy-8-[(3S,4R)-3-hydroxy-1-methylpiperidin-4-yl]chromen-4-one. In some embodiments, from about 10 mg/m² to about 100 mg/m², from about 25 mg/m² to about 60 mg/m², from about 75 mg/m² to about 100 mg/m², about 50 mg/m² or about 90 mg/m² alvocidib, or a pharmaceutically acceptable salt thereof, is administered to the subject per day.

Typically, alvocidib, or a pharmaceutically acceptable salt is administered intravenously. In some embodiments, alvocidib, or a pharmaceutically acceptable salt thereof, is administered by intravenous bolus of from about 10 minutes to about 60 minutes, from about 15 minutes to about 45 minutes or about 30 minutes in duration. When alvocidib, or a pharmaceutically acceptable salt thereof, is administered by intravenous bolus, typically from about 5 mg/m² to about 50 mg/m², from about 20 mg/m² to about 30 mg/m², from about 25 mg/m² to about 35 mg/m² or from about 25 mg/m² to about 60 mg/m² (e.g., about 25 mg/m², about 30 mg/m², about 50 mg/m²) is administered in the bolus. In some embodiments, about 30 mg/m² alvocidib, or a pharmaceutically acceptable salt thereof, is administered by intravenous bolus, for example, once daily for three consecutive days, for example, on a 28-day cycle. In some embodiments, from about 20 mg/m² to about 30 mg/m² (e.g., about 20 mg/m², about 30 mg/m²) alvocidib, or a pharmaceutically acceptable salt thereof, is administered by intravenous bolus, for example, once per treatment cycle, for example, on day 8 or day 10 of the treatment cycle. In some embodiments, about 50 mg/m² alvocidib, or a pharmaceutically acceptable salt thereof, is administered by intravenous bolus, for example, once weekly (e.g., once weekly for three consecutive weeks, for example, on a 28-day cycle). In some embodiments, about 25 mg/m² alvocidib, or a pharmaceutically acceptable salt thereof, is administered once by intravenous bolus, for example, on day 1 of a 28-day treatment cycle, and 50 mg/m² alvocidib, or a pharmaceutically acceptable salt thereof, is administered once by intravenous bolus, for example, on day 15 of the 28-day treatment cycle. In some embodiments (e.g., wherein alvocidib, or a pharmaceutically acceptable salt thereof, is administered as a monotherapy), about 25 mg/m² alvocidib, or a pharmaceutically acceptable salt thereof, is administered once by intravenous bolus, for example, on day 1 of a 28-day treatment cycle, and 50 mg/m² alvocidib, or a pharmaceutically acceptable salt thereof, is administered twice by intravenous bolus, for example, on days 8 and 15 of the 28-day treatment cycle.

In some embodiments, alvocidib, or a pharmaceutically acceptable salt thereof, is administered by intravenous infusion of from about 3 hours to about 5 hours, from about 3.5 hours to about 4.5 hours or about 4 hours (e.g., ±30 minutes) in duration. In some embodiments, alvocidib, or a pharmaceutically acceptable salt thereof, is administered by infusion of from about 30 minutes to about one hour in duration. In some embodiments, alvocidib, or a pharmaceutically acceptable salt thereof, is administered by infusion of about one hour in duration (e.g., one hour ±15 minutes). When alvocidib, or a pharmaceutically acceptable salt thereof, is administered by intravenous infusion, typically from about 10 mg/m² to about 100 mg/m², from about 25 mg/m² to about 90 mg/m², from about 10 mg/m² to about 65 mg/m², from about 30 mg/m² to about 60 mg/m², from about 80 mg/m² to about 100 mg/m² (e.g., about 90 mg/m²) or from about 50 mg/m² to about 75 mg/m² (e.g., about 25 mg/m², about 30 mg/m², about 40 mg/m², about 50 mg/m², about 60 mg/m²) is administered in the infusion. In some embodiments, about 60 mg/m² alvocidib, or a pharmaceutically acceptable salt thereof, is administered by intravenous infusion, for example, once daily for three consecutive days, for example, on a 28-day cycle. In some embodiments, from about 30 mg/m² to about 60 mg/m² (e.g., about 30 mg/m², about 40 mg/m², about 50 mg/m², about 60 mg/m²) alvocidib, or a pharmaceutically acceptable salt thereof, is administered by intravenous infusion, for example, once per treatment cycle. In some embodiments, from about 80 mg/m² to about 100 mg/m² (e.g., about 90 mg/m²) alvocidib, or a pharmaceutically acceptable salt thereof, is administered by intravenous infusion, for example, once per treatment cycle. In some embodiments, from about 25 mg/m² to about 90 mg/m² alvocidib, or a pharmaceutically acceptable salt thereof, is administered by intravenous infusion, for example, weekly or on day 8 when administration of alvocidib, or a pharmaceutically acceptable salt thereof, follows administration of a hypomethylating agent, such as azacitidine or decitabine, or a pharmaceutically acceptable salt of the foregoing.

In some embodiments, alvocidib, or a pharmaceutically acceptable salt thereof, is administered by intravenous bolus, for example, as described herein, and intravenous infusion, for example, as described herein. When alvocidib, or a pharmaceutically acceptable salt thereof, is administered by intravenous bolus and intravenous infusion, the bolus typically precedes the intravenous infusion. In some embodiments, an intravenous infusion of alvocidib, or a pharmaceutically acceptable salt thereof, is initiated within about one hour (e.g., within about 45 minutes, within about 30 minutes) of completion of the bolus of alvocidib, or a pharmaceutically acceptable salt thereof. In some embodiments, an intravenous infusion of alvocidib, or a pharmaceutically acceptable salt thereof, is initiated about 30 minutes after completion of a bolus of alvocidib, or a pharmaceutically acceptable salt thereof. In some embodiments, about 30 mg/m² alvocidib, or a pharmaceutically acceptable salt thereof, is administered by intravenous bolus, and then about 60 mg/m² alvocidib, or a pharmaceutically acceptable salt thereof, is administered by intravenous infusion. Administration of a so-called hybrid dose of alvocidib, or a pharmaceutically acceptable salt thereof (a dose administered by intravenous bolus and intravenous infusion), can occur according to any one of the treatment cycles and/or dosing schedules described herein.

In some embodiments, an effective and/or therapeutically effective amount of a prodrug of alvocidib, or a pharmaceutically acceptable salt thereof, is administered to a subject.

“Prodrug” is meant to indicate a compound that may be converted under physiological conditions or by solvolysis to a biologically active compound. Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammal (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein for their teachings regarding the same. The term “prodrug” is also meant to include any covalently bonded carriers, which release the active compound in vivo when such prodrug is administered to a subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound, and the like.

Examples of prodrugs of alvocidib are described in International Publication Nos. WO 2016/187316 and WO 2018/094275, which are incorporated herein by reference in their entireties for their teachings regarding the same. In some embodiments, the prodrug of alvocidib is a phosphate prodrug of alvocidib. In some instances, the prodrug of alvocidib can be a compound of structural formula I:

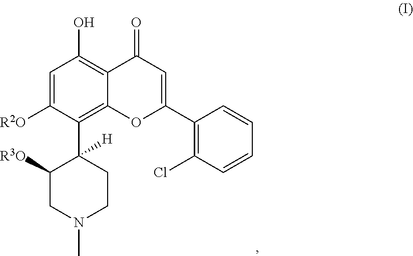

or a pharmaceutically acceptable salt thereof, wherein one of R1, R2 and R3 is —P(═O)(OH)2, and the other two of R1, R2 and R3 are each —H. In some instances, the prodrug of alvocidib can be the compound of structural formula Ia:

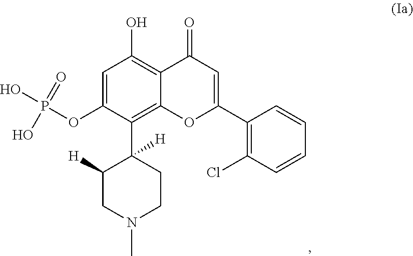

or a pharmaceutically acceptable salt thereof. The compounds of Structural Formulas I and Ia are orally bioavailable. Thus, the compounds of Structural Formulas I and Ia, or a pharmaceutically acceptable salt of the foregoing, can be administered orally, and compositions comprising a compound of Structural Formula I or Ia, or a pharmaceutically acceptable salt thereof, can be formulated for oral administration.

It will be appreciated that a prodrug of alvocidib, such as the compound of structural formula Ia, can exist in zwitterionic form, such as the zwitterionic form represented by structural formula Ib:

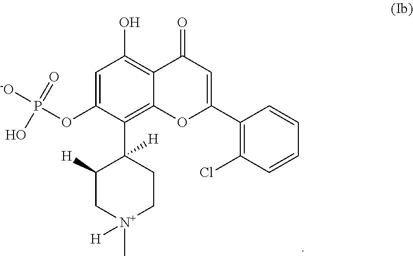

In any of the embodiments of a prodrug herein, the prodrug (e.g., compound of structural formula Ia) can be present in its free form or zwitterionic form, or a pharmaceutically acceptable salt form. Thus, in some embodiments, the prodrug is a compound of structural formula Ia, or a zwitterionic form or pharmaceutically acceptable salt thereof, e.g., a compound of structural formula Ib.

A prodrug of alvocidib (e.g., a compound of Structural Formula I or Ia), or a pharmaceutically acceptable salt thereof is effective over a wide dosage range. For example, in the treatment of adult humans, dosages from about 0.01 mg to about 1000 mg, from about 0.5 mg to about 100 mg, from about 0.5 mg to about 100 mg, from about 1 mg to about 50 mg per day, and from about 5 mg to about 40 mg per day are examples of dosages that are used in some embodiments. In particular embodiments, the dosage ranges from about 1 mg to about 60 mg (e.g., from about 5 mg to about 60 mg, from about 10 mg to about 60 mg, from about 5 mg to about 50 mg, from about 10 mg to about 30 mg, from about 10 mg to about 50 mg, from about 20 to about 50 mg, from about 25 mg to about 45 mg) per day. In other embodiments, the dosage is from about 1 mg to about 30 mg per day, e.g., about 1 mg, about 2 mg, about 4 mg, about 8 mg, about 12 mg, about 16 mg, about 20 mg, about 22 mg, about 24 mg, about 26 mg, about 28 mg, about 30 mg or about 32 mg per day (e.g., administered QD, administered BID). In other embodiments, the dosage is from about 1 mg to about 30 mg, e.g., about 1 mg, about 2 mg, about 4 mg, about 6 mg, about 8 mg, about 11 mg, about 12 mg, about 16 mg, about 20 mg, about 22 mg, about 24 mg, about 26 mg, about 28 mg or about 30 mg, administered BID. The exact dosage will depend, for example, upon the route of administration, the form in which the prodrug is administered, the subject to be treated, the body weight of the subject to be treated, and the preference and experience of the attending physician.

In some embodiments, the prodrug of alvocidib (e.g., a compound of Structural Formula I or Ia), or a pharmaceutically acceptable salt thereof, is administered to the subject orally, for example, in an amount of from about 0.5 mg to about 5 mg per day. In some embodiments, about 1 mg or about 2 mg of a prodrug of alvocidib (e.g., a compound of Structural Formula I or Ia), or a pharmaceutically acceptable salt thereof, is administered to a subject twice a day, or about 1 mg or about 2 mg of a prodrug of alvocidib (e.g., a compound of Structural Formula I or Ia), or a pharmaceutically acceptable salt thereof, is administered to a subject once a day.

As used herein, “pharmaceutically acceptable salts” refers to salts derived from suitable inorganic and organic acids and bases that are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable acid addition salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salts.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, or copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Examples of organic amines include, but are not limited to, isopropylamine, benzathine, cholinate, diethanolamine, diethylamine, lysine, meglumine, piperazine and tromethamine.

Pharmaceutically acceptable salts can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

A subject is considered to be in “complete remission” or “CR” if less than 5% leukemic blasts are present in the subject's bone marrow. Typically, a subject in CR has an absence of blasts and blasts with Auer rods, an absence of extramedullary disease, an absolute neutrophil count ≥1.0×10⁹/L (1,000/μL) and a platelet count of >100×10⁹/L (100,000/μL). As used herein, complete remission includes complete remission with partial hematological recovery (CRᵢ), meaning the subject meets the CR requirement for blast count, but exhibits residual neutropenia [<1.0×10⁹/L (1,000/μL)] or thrombocytopenia [<100×10⁹/L (100,000/μL)].

In some embodiments, the subject is MRD-negative following administration of alvocidib, or a prodrug thereof, or a pharmaceutically acceptable salt of the foregoing.

Measurable residual disease, minimal residual disease and MRD refer to the post-therapy persistence of leukemic cells at levels below morphologic detection. Although not wishing to be bound by any particular theory, MRD is thought to be a strong prognostic indicator of increased risk of relapse or shorter survival in patients with hematologic cancers, such as AML. MRD testing is typically conducted using one of three techniques: immunophenotypic detection by multiparameter flow cytometry (MFC), real-time quantitative PCR (RT-qPCR) and next-generation sequencing technology. MFC uses panels of fluorochrome-labeled monoclonal antibodies to identify aberrantly expressed antigens of leukemic cells. RT-qPCR can be used to amplify leukemia-associated genetic abnormalities. Next-generation sequencing technology can be used to evaluate a few genes or an entire genome. Together, RT-qPCR and next-generation sequencing technology represent molecular approaches to MRD testing. Representative methods of detecting MRD status in an AML subject are described in Ravandi, F., et al., Blood Advances 12 Jun. 2018, vol. 2, no. 11, and Schuurhuis, G. J., et al., Blood 2018 Mar. 22, 131(12): 1275-1291, the relevant contents of which are incorporated herein by reference in their entireties. Representative methods of detecting MRD status in a MM subject are described in Gambella, M. et al., Cancer 1 Mar. 2019, 750-760; Perrot, A., Blood 6 Dec. 2018, 132(23): 2456-2464; Landgren, O., Seminars in Hematology, 55(2018): 1-3; Landgren, O., et al., Seminars in Hematology 55(2018):44-50; Thoren, K. L., Seminars in Hematology 55(2018):41-43; Pugh, T. J., Seminars in Hematology 55(2018):38-40; Waldschmidt, J. M., Seminars in Hematology 55(2018):33-37; Pandit-Taskar, N., Seminars in Hematology 55(2018):22-32; Hillengass, J., Seminars in Hematology 55(2018):19-21; and Roshal, M., Seminars in Hematology 55(2018):4-12, the relevant contents of which are incorporated herein by reference in their entireties. The U.S. Department of Health and Human Services, Food and Drug Administration, Oncology Center of Excellence (OCE), Center for Drug Evaluation and Research (CDER) and Center for Biologics Evaluation and Research (CBER) have also published guidance for sponsors planning to use MRD as a biomarker in clinical trials conducted under an investigational new drug application (IND) or to support marketing approval of drugs and biological products for treating specific hematologic malignancies in “Hematologic Malignancies: Regulatory Considerations for Use of Minimal Residual Disease in Development of Drug and Biological Products for Treatment Guidance For Industry,” January 2020, available from https://www.fda.gov/media/134605/download, the relevant contents of which are incorporated herein by reference in their entireties.

To guide the development of a standardized approach to MRD testing, the European LeukemiaNet (ELN) has issued consensus recommendations for the measurement of MRD in AML. According to the ELN, a percentage of AML cells to leukocytes of 0.1% or greater in a subject's bone marrow, measured by MFC according to the ELN's recommendations for MRD testing by MFC, indicates the subject is MRD positive (MRD+) by MFC according to the ELN's recommendations for MRD testing by MFC. A percentage of AML cells to leukocytes of less than 0.1% in a subject's bone marrow, measured by MFC according to the ELN's recommendations for MRD testing by MFC, indicates the subject is MRD negative (MRD−) by MFC according to the ELN's recommendations for MRD testing by MFC.

The ELN has also issued guidelines for molecular MRD testing in AML. The ELN defines complete molecular remission as complete morphologic remission plus two successive negative MRD samples obtained within an interval of ≥4 weeks at a sensitivity level of at least 1 in 1,000 (e.g., 1 in 1,000; 1 in 10,000), wherein the samples are collected and measured according to the ELN guidelines for molecular MRD testing. The ELN defines molecular persistence at low copy numbers, which is associated with a low risk of relapse, as MRD with low copy numbers (<100-200 copies/10⁴ ABL copies corresponding to <1-2% of target to reference gene or allele burden) in patients with morphologic CR, and a copy number or relative increase <1 log between any two positive samples collected at the end of treatment, wherein the samples are collected and measured according to the ELN guidelines for molecular MRD testing. The ELN defines molecular progression in patients with molecular persistence as an increase of MRD copy numbers >1 log 10 between any two positive samples collected and measured according to the ELN guidelines for molecular MRD testing. The ELN defines molecular relapse as an increase of the MRD level of >1 log 10 between two positive samples in a patient who previously tested negative, wherein the samples are collected and measured according to the ELN guidelines for molecular MRD testing. Both molecular persistence and molecular relapse are indicators of an MRD-positive subject by RT-qPCR conducted according to the ELN guidelines for MRD testing by RT-qPCR. Thus, patients in complete molecular remission and patients labelled as having molecular persistence at low copy numbers are MRD-negative by RT-qPCR conducted according to the ELN guidelines for MRD testing by RT-qPCR. RT-qPCR is the recommended molecular approach to MRD testing, as discussed in Ravandi, F., et al. and Schuurhuis, G. J., et al. Specific recommendations for collecting and measuring samples (e.g., bone marrow samples) for MRD testing are described in Ravandi, F., et al., Blood Advances 12 Jun. 2018, vol. 2, no. 11 and Schuurhuis, G. J., et al., Blood 2018 Mar. 22, 131(12): 1275-1291, the relevant contents of which are incorporated herein by reference in their entireties.

When a subject having AML is described herein as being “measurable residual disease negative,” “minimal residual disease negative,” “MRD-negative” or “MRD⁻” without a further modifier, such as by MFC or by RT-qPCR, the subject is MRD negative according to at least one of the ELN's criteria described herein (e.g., MFC, molecular biology). In some embodiments, the subject is MRD-negative by MFC conducted according to ELN guidelines for MRD testing. In some embodiments, the subject is MRD-negative by RT-qPCR conducted according to ELN guidelines for MRD testing. In some embodiments, the subject is MRD-negative by both MFC and RT-qPCR conducted according to ELN guidelines for MRD testing. In some embodiments, the subject is MRD-negative by MFC conducted according to ELN guidelines for MRD testing, and is MRD-positive by RT-qPCR conducted according to ELN guidelines for MRD testing. In some embodiments, the subject is MRD-positive by MFC conducted according to ELN guidelines for MRD testing, and is MRD-negative by RT-qPCR conducted according to ELN guidelines for MRD testing. When a subject is MRD-negative according to one of the ELN's criterion described herein (e.g., the criterion for MFC), but MRD-positive according to another of the ELN's criterion described herein (e.g., the criterion for RT-qPCR), that subject can still be described as MRD-negative according to the use of that term herein because the subject is MRD negative according to at least one of the ELN's criteria described herein.

When a subject having AML, is described herein as being “measurable residual disease positive,” “minimal residual disease positive,” “MRD-positive” or “MRD+,” the subject is MRD positive by the ELN's criteria for MFC and RT-qPCR described herein. For example, a subject that is MRD positive for AML can be MRD-positive by MFC conducted according to ELN guidelines for MRD testing in AML, and MRD-positive by RT-qPCR conducted according to ELN guidelines for MRD testing in AML.

Other organizations have also put forth guidelines for classifying patients as MRD positive and/or MRD negative. For example, NCCN, ASCO and ESMO have put forth guidelines for classifying patients having AML as MRD positive or MRD negative. NCCN's guidelines state that MRD in AML refers to the presence of leukemic cells below the threshold of detection by conventional morphologic methods. MRD is a component of patient evaluation over the course of sequential therapy. Patients who achieved a CR by morphologic assessment alone can still harbour a large number of leukemic cells in the bone marrow. Methods of detection are: RQ-PCR and flow cytometry using a standard MRD assay. RQ-PCR has a detection range of 1 in 1000 to 1 in 100,000, while flow cytometry has sensitivity between 10⁻⁴ to 10⁻⁵. A negative MRD result after induction predicts a lower incidence of relapse, but is not a proof of relapse. However, a persistently positive MRD result after induction is associated with an increased risk of relapse. The timing of MRD assessments will vary, and depend on the regimen used, but may occur after completion of initial induction and before allogenic transplantation. See https://www.nccn.org/professionals/physician_gls/pdf/aml.pdf. ASCO's guidelines state that emerging evidence supports molecular studies as principle tests for monitoring MRD of AML. The key molecular markers included in monitoring MRD are PML-RARA, RUNX1-RUNXT1, CBFBMYH11, NPM1, CEBPA, RUNX1, and KIT. However, it is unclear whether a large screening panel should be applied for MRD detection. Also, a recent consensus from the ELN MRD working group proposed that for detection of molecular MRD, the real-time quantitative PCR platform is preferred to NGS and digital PCR platforms. The latter must be further validated. See https://ascopubs.org/doi/pdf/10.1200/JCO.18.01468. ESMO's guidelines state that MRD negativity is defined as negativity for a genetic marker by a validated molecular method, or negativity by MFC. An increase of the MRD level of ≥1 log 10 between two positive samples in a patient who previously tested negative is considered molecular relapse. See https://www.esmo.org/guidelines/haematological-malignancies/acute-myeloid-leukaemia.

In some embodiments, a subject having AML is MRD-negative or MRD-positive according to NCCN guidelines. In some embodiments, a subject having AML is MRD-negative or MRD-positive according to ASCO guidelines. In some embodiments, a subject having AML is MRD-negative or MRD-positive according to ESMO guidelines.

For example, NCCN, ASCO and ESMO have put forth guidelines for classifying patients having ALL as MRD positive or MRD negative. NCCN's guidelines state that MRD in ALL refers to the presence of leukemic cells below the threshold of detection by conventional morphologic methods. MRD is a component of patient evaluation over the course of sequential therapy. Patients who achieved a CR by morphologic assessment alone can still harbour a large number of leukemic cells in the bone marrow, up to 10¹⁰ malignant cells. Studies in both children and adults with ALL have demonstrated the strong correlation between MRD and risk of relapse as well as prognostic significance of MRD measurements during and after induction therapy. MRD is also defined by the appearance of BCR-ABL transcripts, as assessed by RT-PCR. Methods of detection are: RQ-PCR and flow cytometry. Current 6-color flow cytometry can detect leukemic cells at a sensitivity threshold of <1×10⁻⁴ (<0.01%) bone marrow MNCs. RQ-PCR methods can detect leukemic cells at a sensitivity threshold of <1×10⁻⁶ (<0.0001%) bone MNCs. Timings for MRD assessment are:
1. Upon completion of initial induction
2. Additional time points should be guided by type of regimen used
3. Serial monitoring frequency may be increased in pateints with molecular relapse or persistent low-level disease burden
4. For some techniques, a baseline sample may be needed or helpful for the MRD assessment to be valid.

See https://www.nccn.org/professionals/physician_gls/pdf/all.pdf. ASCO's guidelines state that emerging evidence supports molecular studies as principle tests for monitoring MRD of AML. The key molecular markers included in monitoring MRD are PML-RARA, RUNX1-RUNXT1, CBFBMYH11, NPM1, CEBPA, RUNX1, and KIT. However, it is unclear whether a large screening panel should be applied for MRD detection. Also, a recent consensus from the ELN MRD working group proposed that for detection of molecular MRD, the real-time quantitative PCR platform is preferred to NGS and digital PCR platforms. The latter must be further validated. See https://ascopubs.org/doi/pdf/10.1200/JCO.18.01468. EMSO's guidelines state that molecular response can be evaluated only for patients in complete cytologic remission with one marker or more for MRD analysis and samples available at the respective time points. If MRD is measured by flow cytometry, a good MRD response is often defined as less than 10⁻³, although MRD levels less than 10⁻⁴ can be achieved with the 8-12-colour flow cytometers. See https://www.esmo.org/guidelines/haematological-malignancies/acute-lymphoblastic-leukaemia.

In some embodiments, a subject having ALL is MRD-negative or MRD-positive according to NCCN guidelines. In some embodiments, a subject having ALL is MRD-negative or MRD-positive according to ASCO guidelines. In some embodiments, a subject having ALL is MRD-negative or MRD-positive according to ESMO guidelines.

For example, NCCN and ESMO have put forth guidelines for classifying patients having CLL as MRD positive or MRD negative. NCCN's guidelines state that MRD evaluation with <10⁻⁴ detectable leukemic cells in peripheral blood and bone marrow after the end of treatment is associated with long term survival. Allele specific oligonucleotide PCR and six color flow cytometry are two validated methods for detection of MRD at the levels of 10⁻⁴ to 10⁻⁵. Next generation DNA sequencing based assay is reported to be more sensitive at the level of 10⁻⁶. See https://www.nccn.org/professionals/physician_gls/pdf/cll.pdf. EMSO's guidelines state that patients who are MRD-negative after therapy show a longer response duration and survival. Additional clinical consequences of MRD positivity post-therapy in CLL remain unclear except for patients after an allogeneic transplantation, where a positive MRD signal may trigger the reduction of immunosuppressive therapies or the start of anti-leukaemic maintenance therapy. Therefore, MRD assessment is not generally recommended for monitoring post-therapy outside clinical studies. See https://www.esmo.org/guidelines/haematological-malignancies/chronic-lymphocytic-leukaemia.

In some embodiments, a subject having CLL is MRD-negative or MRD-positive according to NCCN guidelines. In some embodiments, a subject having CLL is MRD-negative or MRD-positive according to ESMO guidelines.

For example, NCCN, ASCO, IMWG and ESMO have put forth guidelines for classifying patients having MM as MRD positive or MRD negative. NCCN guidelines state that criteria for MRD are:
1. Sustained MRD-negative: MRD negativity in the marrow, confirmed minimum of 1 year apart. Subsequent evaluations can be used to further specify the duration of negativity (e.g., MRD-negative at 5 years)
2. Flow MRD-negative: Absence of phenotypically aberrant clonal plasma cells by NGF on bone marrow aspirates using EuroFlow standard operation procedure for MRD detection in multiple myeloma with a minimum sensitivity of 1 in 10⁵ nucleated cells or higher
3. Sequencing MRD-negative: Absence of clonal plasma cells by NGS on bone marrow aspirate in which presence of a clone is defined as less than two identical sequencing reads obtained after DNA sequencing of bone marrow aspirates using a validated equivalent method with a minimum sensitivity of 1 in 10⁵ nucleated cells or higher
4. Imaging plus MRD-negative: MRD negativity as defined by NGF or NGSplus disappearance of every area of increased tracer uptake found at baseline or a preceding FDG PET/CT or decrease to less mediastinal blood pool SUV or decrease to less than that of surrounding normal tissue.

See https://www.nccn.org/professionals/physician_gls/pdf/myeloma.pdf. ASCO's guidelines state that MRD can be detected using several techniques. Next generation flow cytometry relies on two eight-color antibody panels targeting cell surface antigens to identify phenotypically aberrant clonal plasma cells and includes detection of cytoplasmic [symbol] and [symbol] , light-chain expression to confirm clonality. It has a sensitivity of 1 in 10⁵ cells or higher. NGS uses sets of multiple polymerase chain reaction primers for the amplification and sequencing of immunoglobulin gene segments. DNA sequencing of bone marrow aspirates using the Lympho-SIGHT platform (or validated equivalent method) has a minimum sensitivity of 1 in 10⁵ nucleated cells or higher. However, there is no universal agreement as to which method is preferred, when the testing should be performed, and at what interval. MRD testing by sequencing requires a baseline sample, whereas NGF does not. See https://ascopubs.org/doi/pdf/10.1200/JCO.18.02096. IMWG's and ESMO's guidelines track NCCN's guidelines. See https://www.thelancet.com/journals/lanonc/article/PIIS1470-2045(16)30206-6/fulltext and https://www.esmo.org/guidelines/haematological-malignancies/multiple-myeloma, respectively.

In some embodiments, a subject having MM is MRD-negative or MRD-positive according to NCCN guidelines. In some embodiments, a subject having MM is MRD-negative or MRD-positive according to ASCO guidelines. In some embodiments, a subject having MM is MRD-negative or MRD-positive according to IMWG guidelines. In some embodiments, a subject having MM is MRD-negative or MRD-positive according to ESMO guidelines.

Some embodiments of the methods described herein include administering the therapeutic agents described herein to the subject based on MRD status. Thus, in some embodiments, administration of alvocidib, or a prodrug thereof, or a pharmaceutically acceptable salt of the foregoing, is continued at least until the subject is MRD-negative. In some embodiments, administration of the maintenance therapy is continued at least until the subject is MRD-negative. In some embodiments, the subject continues to receive at least one of one or more therapeutic agents from the prior therapy (e.g., in combination with alvocidib, or a prodrug thereof, or a pharmaceutically acceptable salt of the foregoing) at least until the subject is MRD-negative.

Typically, however, administration of alvocidib, or a prodrug thereof, or a pharmaceutically acceptable salt of the foregoing, or a maintenance therapy, or continuation of one or more therapeutic agents from a prior therapy will continue for a period of time after the subject becomes and/or is appreciated to be (e.g., by detection of MRD status as described herein) MRD-negative. More typically, administration of alvocidib, or a prodrug thereof, or a pharmaceutically acceptable salt of the foregoing, or a maintenance therapy, or continuation of one or more therapeutic agents from a prior therapy (e.g., in combination with alvocidib, or a prodrug thereof, or a pharmaceutically acceptable salt of the foregoing) will continue for a period of time after the subject becomes and/or is appreciated to be MRD-negative as long as the subject continues to be MRD-negative for the period of time. Representative periods of time include at least or about 30 days, at least or about 60 days, at least or about 90 days, at least or about 180 days, at least or about one year, at least or about two years, at least or about three years, at least or about four years, or at least or about five years. Thus, for example, administration of alvocidib, or a prodrug thereof, or a pharmaceutically acceptable salt of the foregoing, and/or a maintenance therapy and/or one or more therapeutic agents from a prior therapy (e.g., in combination with alvocidib, or a prodrug thereof, or a pharmaceutically acceptable salt of the foregoing) can be continued for about two years after a subject is determined to be MRD-negative, if the subject continues to be MRD-negative for those two years.

Some embodiments further comprise detecting the MRD status of the subject (e.g., prior to administering alvocidib, or a prodrug thereof, or a pharmaceutically acceptable salt of the foregoing and/or after administering alvocidib, or a prodrug thereof, or a pharmaceutically acceptable salt of the foregoing). In some embodiments, the MRD status of the subject is detected prior to administering alvocidib, or a prodrug thereof, or a pharmaceutically acceptable salt of the foregoing. Detection of the subject's MRD status prior to treatment can be used to determine whether administration of alvocidib, or a prodrug thereof, or a pharmaceutically acceptable salt of the foregoing, is indicated, or whether a subsequent treatment is efficacious (e.g., as a baseline measurement). For example, detection of a positive MRD status in a subject may indicate that treatment according to a method disclosed herein is indicated, despite the subject being in complete remission. Alternatively, detection of a negative MRD status may indicate that the subject is not a suitable candidate for treatment according to a method disclosed herein.

In some embodiments, the MRD status of the subject is detected after administering alvocidib, or a prodrug thereof, or a pharmaceutically acceptable salt of the foregoing, to the subject. Detection of the subject's MRD status after administration of alvocidib, or a prodrug thereof, or a pharmaceutically acceptable salt of the foregoing, can be used, for example, to determine whether to terminate a therapy or to track the progress of a subject on a therapy. For example, detection of a positive MRD status in a subject may indicate that continued therapy (e.g., another cycle of therapy) according to a method disclosed herein is indicated, despite the subject having completed a cycle of therapy. Alternatively, detection of a negative MRD status may indicate that therapy may be terminated.

In some embodiments, the MRD status of the subject is detected prior to and after administering alvocidib, or a prodrug thereof, or a pharmaceutically acceptable salt of the foregoing, to the subject.

Because MRD is believed to be a useful clinical endpoint, detection of MRD negative status in a subject is a useful clinical finding. Accordingly, in some embodiments of the methods described herein, the method further comprises terminating administration of alvocidib, or a prodrug thereof, or a pharmaceutically acceptable salt of the foregoing and/or a maintenance therapy and/or one or more therapeutic agents of a prior therapy, if the subject is determined to be MRD-negative. In some embodiments, the method further comprises terminating administration of alvocidib, or a prodrug thereof, or a pharmaceutically acceptable salt of the foregoing and/or a maintenance therapy and/or one or more therapeutic agents of a prior therapy, after a period of time after the subject is determined to be MRD-negative (e.g., if the subject continues to be MRD-negative for the period of time). Representative periods of time include at least or about 90 days, at least or about 180 days, at least or about one year, at least or about two years, at least or about three years, at least or about four years, or at least or about five years. Thus, for example, administration of alvocidib, or a prodrug thereof, or a pharmaceutically acceptable salt of the foregoing and/or a maintenance therapy and/or one or more therapeutic agents of a prior therapy can be terminated about two years after a subject is determined to be MRD-negative, if the subject continues to be MRD-negative for those two years.

It is anticipated that there will be reticence by treating physicians to remove subjects in complete remission from a therapy (e.g., prior therapy, induction therapy) before administering alvocidib, or a prodrug thereof, or a pharmaceutically acceptable salt of the foregoing. Accordingly, the methods described herein contemplate embodiments wherein a subject continues to receive at least one (e.g., one, two, three or all) of the therapeutic agents from a prior therapy (e.g., lenalidomide, or a pharmaceutically acceptable salt thereof) while being administered alvocidib, or a prodrug thereof, or a pharmaceutically acceptable salt of the foregoing, for example, for at least a portion of the time (e.g., the entire time) the subject is being administered alvocidib, or a prodrug thereof, or a pharmaceutically acceptable salt of the foregoing. In some embodiments, a subject continues to receive the prior therapy (e.g., lenalidomide, or a pharmaceutically acceptable salt thereof) while being administered alvocidib, or a prodrug thereof, or a pharmaceutically acceptable salt of the foregoing, for example, for at least a portion of the time (e.g., the entire time) the subject is being administered alvocidib, or a prodrug thereof, or a pharmaceutically acceptable salt of the foregoing, as, for example, when alvocidib, or a prodrug thereof, or a pharmaceutically acceptable salt of the foregoing, is added to an ongoing maintenance therapy. Typically, when alvocidib, or a prodrug thereof, or a pharmaceutically acceptable salt of the foregoing, is added to a maintenance therapy or a continuous therapy intended to bring about complete remission, the subject continues to receive the prior therapy or one or more therapeutic agents thereof without alteration. Thus, for example, the subject continues to receive the prior therapy at the same doses, via the same route of administration and on the same schedule as the subject was receiving the prior therapy or one or more therapeutic agents thereof prior to the addition of alvocidib, or a prodrug thereof, or a pharmaceutically acceptable salt of the foregoing. Adjustments to the doses and the schedule can be made, however, for example, if clinically indicated.

In some embodiments, however, the subject is not receiving a therapeutic agent from the prior therapy while being administered alvocidib, or a prodrug thereof, or a pharmaceutically acceptable salt of the foregoing, as, for example, when alvocidib, or a prodrug thereof, or a pharmaceutically acceptable salt of the foregoing, is administered as an independent therapy following completion of a prior and/or induction therapy.

Administration of the therapeutic agents can be in pure form or in an appropriate pharmaceutical composition, via any of the accepted modes of administration of therapeutic agents for serving similar utilities. For example, a therapeutic agent can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the therapeutic agent and the particular disease to be treated. Administration can be local or systemic as indicated. The preferred mode of administration can vary depending on the particular therapeutic agent chosen. In some embodiments, the therapeutic agent (e.g., compound of Structural Formula I or Ia, or a pharmaceutically acceptable salt thereof) is administered orally. In some embodiments, the therapeutic agent (e.g., alvocidib) is administered intravenously.

### Compositions, Combinations and Kits

The therapeutic agents described herein (e.g., alvocidib, or a prodrug thereof, or a pharmaceutically acceptable salt of the foregoing; lenalidomide, or a pharmaceutically acceptable salt thereof, etc.) can be administered in pure form or in an appropriate pharmaceutical composition comprising one or more therapeutic agents (e.g., a pharmaceutical combination), and one or more pharmaceutically acceptable carriers.

A “pharmaceutically acceptable carrier” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012)).

Typically, pharmaceutically acceptable carriers are sterile. The pharmaceutical composition can be formulated for particular routes of administration such as oral administration, parenteral administration (e.g., intravenous administration) and rectal administration, etc. In addition, the pharmaceutical composition can be made up in a solid form (including, without limitation, capsules, tablets, pills, granules, powders or suppositories), or in a liquid form (including, without limitation, solutions, suspensions or emulsions). The pharmaceutical compositions can be subjected to conventional pharmaceutical operations, such as sterilization, and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.
   Tablets may be either film-coated or enteric-coated according to methods known in the art.

Suitable compositions for oral administration include a therapeutic agent described herein (e.g., a compound of Structural Formula I or Ia, or a pharmaceutically acceptable salt of the foregoing) in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

A pharmaceutical composition for use in the present methods may be in the form of a liquid, for example, an elixir, syrup, solution, emulsion or suspension. The liquid may be for oral administration, such as for compositions comprising a prodrug of alvocidib, or a pharmaceutically acceptable salt thereof, and/or lenalidomide, or a pharmaceutically acceptable salt thereof, or for delivery by injection, such as form compositions comprising alvocidib, or a pharmaceutically acceptable salt thereof, for example. When intended for oral administration, pharmaceutical compositions contain, for example in addition to the therapeutic compound(s), one or more of a sweetening agent, preservative, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably, physiological saline, Ringer's solution, isotonic sodium chloride, fixed oils such as synthetic mono- and diglycerides which may serve as the solvent or suspending medium, polyethylene glycols, glycerin, propylene glycol and other solvents; antibacterial agents such as benzyl alcohol and methyl paraben; antioxidants such as ascorbic acid and sodium bisulfite; chelating agents such as ethylenediaminetetraacetic acid; buffers such as acetates, citrates and phosphates; and agents for the adjustment of tonicity, such as sodium chloride and dextrose. A parenteral preparation can be enclosed in ampoules, disposable syringes or multiple dose vials made of glass or plastic. Physiological saline is a preferred adjuvant. An injectable pharmaceutical composition is preferably sterile. In embodiments, the pharmaceutical composition is formulated for injection. In some embodiments, the pharmaceutical composition is formulated for bolus injection. In embodiments, the pharmaceutical composition is formulation for infusion.

Certain injectable compositions comprise a therapeutic agent described herein (e.g., alvocidib, or a pharmaceutically acceptable salt thereof; lenalidomide, or a pharmaceutically acceptable salt thereof) in the form of an aqueous isotonic solution or suspension, and certain suppositories comprising a therapeutic agent described herein are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include a therapeutic agent described herein with a suitable carrier. Carriers suitable for transdermal delivery include absorbable pharmacologically acceptable solvents to assist passage through the skin of the host. For example, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

Suitable compositions comprising a therapeutic agent described herein for topical application, e.g., to the skin and eyes, include aqueous solutions, suspensions, ointments, creams, gels or sprayable formulations, e.g., for delivery by aerosol or the like. Such topical delivery systems will, in particular, be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein, a topical application may also pertain to an inhalation or to an intranasal application. A composition suitable for inhalation or intranasal administration may be conveniently delivered in the form of a dry powder (either alone, as a mixture, for example a dry blend with lactose, or a mixed component particle, for example, with phospholipids) from a dry powder inhaler, or an aerosol spray presentation from a pressurised container, pump, spray, atomizer or nebuliser, with or without the use of a suitable propellant.

A therapeutic agent described herein can also be provided in anhydrous pharmaceutical compositions and dosage forms, since water may facilitate the degradation of certain compounds. Anhydrous pharmaceutical compositions and dosage forms can be prepared using anhydrous or low moisture-containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Pharmaceutical compositions and dosage forms can also comprise one or more agents that reduce the rate by which a therapeutic agent will decompose. Such agents, which are referred to herein as “stabilizers,” include, but are not limited to, antioxidants such as ascorbic acid, pH buffers, or salt buffers, etc.

A pharmaceutical composition used in certain embodiments of the disclosure may be prepared by methodology well known in the pharmaceutical art. For example, a pharmaceutical composition intended to be administered by injection can be prepared by combining one or more of the therapeutic agents with sterile, distilled water so as to form a solution. In some embodiments, pharmaceutical composition(s) for administration according to methods of the disclosure take the form of a liquid where the therapeutic agents are present in solution, in suspension, or both. In some embodiments, when a therapeutic agent is administered as a solution or suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of the therapeutic agents. The term “solubilizing agent” generally includes agents that result in formation of a micellar solution or a true solution of the agent. Certain acceptable nonionic surfactants, for example polysorbate 80, are useful as solubilizing agents, as are ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

A surfactant may be added to facilitate the formation of a homogeneous solution or suspension. Surfactants are compounds that non-covalently interact with a therapeutic agent so as to facilitate dissolution or homogeneous suspension. In embodiments, a pharmaceutical composition includes one or more surfactants to enhance physical stability. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

A pharmaceutical composition for use in embodiments of the disclosure may include various materials that modify the physical form of a solid or liquid dosage unit. For example, the composition may include materials that form a coating shell around one or more of the therapeutic agents. The materials that form the coating shell are typically inert, and may be selected from, for example, sugar, shellac, and other enteric coating agents. Alternatively, the active ingredients may be encased in a gelatin capsule.

A pharmaceutical composition used in certain embodiments may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of the therapeutic agents may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

A therapeutic agent described herein is typically formulated into pharmaceutical dosage forms to provide an easily controllable dosage of the drug and to give the patient an elegant and easily handleable product. The dosage regimen will, of course, vary depending upon known factors, such as the pharmacodynamic characteristics of the particular therapeutic agent and its mode and route of administration; the species, age, sex, health, medical condition, and weight of the recipient; the nature and extent of the symptoms; the kind of concurrent treatment; the frequency of treatment; the route of administration; the renal and hepatic function of the patient; and the effect desired. Therapeutic agents described herein may be administered in a single daily dose, or the total daily dosage may be administered in divided doses, e.g., two, three, or four times daily.

Compositions for use in combination therapies will either be formulated together as a pharmaceutical combination, or provided for separate administration (e.g., associated in a kit). Accordingly, a further embodiment is a pharmaceutical combination comprising two or more therapeutic agents described herein. A pharmaceutical combination can further comprise one or more pharmaceutically acceptable carriers, such as one or more of the pharmaceutically acceptable carriers described herein.

A pharmaceutical composition can be in a unit dosage containing from about 1 to about 1000 mg of active ingredient(s) for a subject of from about 50 to about 70 kg, or from about 1 to about 500 mg, from about 1 to about 250 mg, from about 1 to about 150 mg, from about 0.5 to about 100 mg, or from about 1 to about 50 mg of active ingredient(s) for a subject of from about 50 to about 70 kg. The effective and/or therapeutically effective dosage of a therapeutic agent/pharmaceutical composition is dependent on the species of the subject, the body weight, age and individual condition of the subject, and the disorder or disease or the severity thereof being treated. A physician, clinician or veterinarian of ordinary skill can readily determine the effective and/or therapeutically effective amount of each of the active ingredients necessary to prevent or treat the progress of the disorder or disease.

In some embodiments, the concentration of one or more therapeutic agents provided in a pharmaceutical composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more therapeutic agents provided in a pharmaceutical composition is greater than 90%, 80%, 70%, 60%, 50%, 40%, 30%, 20%, 19.75%, 19.50%, 19.25%, 19%, 18.75%, 18.50%, 18.25%, 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in a pharmaceutical composition is in the range from about 0.0001% to about 50%, about 0.001% to about 40%, about 0.01% to about 30%, about 0.02%, to about 29%, about 0.03%, to about 28%, about 0.04%, to about 27%, about 0.05% to about 26%, about 0.06%, to about 25%, about 0.07%, to about 24%, about 0.08%, to about 23%, about 0.09%, to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8%, to about 14%, about 0.9%, to about 12%, about 1% to about 10% w/w, w/v or v/v.

In some embodiments, the concentration of one or more therapeutic agents provided in a pharmaceutical composition is in the range from about 0.001%, to about 10%, about 0.01%, to about 5%, about 0.02%, to about 4.5%, about 0.03%, to about 4%, about 0.04% to about 3.5%, about 0.05% to about 3%, about 0.06%, to about 2.5%, about 0.07% to about 2%, about 0.08% to about 1.5%, about 0.09% to about 1%, about 0.1% to about 0.9% w/w, w/v or v/v.

Another embodiment is a kit comprising two or more, separate therapeutic agents (e.g., two or more, separate pharmaceutical compositions). In one embodiment, the kit comprises an effective and/or therapeutically effective amount of each therapeutic agent (e.g., each pharmaceutical composition). For example, in some embodiments, a kit comprises alvocidib, or prodrug thereof (e.g., a compound of Structural Formula Ia), or a pharmaceutically acceptable salt of the foregoing, and an additional therapeutic agent (e.g., lenalidomide, or a pharmaceutically acceptable salt thereof; lenalidomide, or a pharmaceutically acceptable salt thereof, and dexamethasone, or a pharmaceutically acceptable salt thereof; VYXEOS®).

The kit of the present disclosure may be used for administering different dosage forms, for example, oral and parenteral, for administering the separate compositions at different dosage intervals, or for titrating the separate compositions against one another.

To assist compliance, a kit typically comprises directions for administration. The written instructions may include instructions regarding dosage, method of administration, order and timing of administration, and the like. The written instructions can be in the form of printed instructions provided within the kit, or the written instructions can be printed on a portion of the container housing the kit. Written instructions may be in the form of a sheet, pamphlet, brochure, CD-ROM, or computer-readable device, or can provide directions to locate instructions at a remote location, such as a website. The written instructions may be in English and/or in a national or regional language.

Kits can further comprise one or more syringes, ampules, vials, tubes, tubing, facemask, a needleless fluid transfer device, an injection cap, sponges, sterile adhesive strips, Chloraprep, gloves, and the like. Variations in contents of any of the kits described herein can be made. In various embodiments, the contents of the kit are provided in a compact container.

In some embodiments, pharmaceutical compositions of the disclosure are presented in a pack or dispenser device that contains one or more unit dosage forms containing the active ingredient(s). The pack may, for example, comprise metal or plastic foil, such as a blister pack.

In embodiments, the kit (e.g., a pack or dispenser) may be accompanied by a notice associated with the container in a form prescribed by a governmental agency regulating the manufacture, use or sale of pharmaceuticals, which notice is reflective of approval by the agency of the form of the compositions or of human or veterinary administration, in addition to instructions for administration. Such notice, for example, may be of the labeling approved by the U.S. Food and Drug Administration for prescription drugs or of an approved product insert.

### EXEMPLIFICATION

### Example 1. MCL-1 Dependency in AML

CD34, CD38 and ALDH are leukemic stem cell (LSC) markers. CD123, TIM3 and CD96 are specific LSC markers that can be used to differentiate LSCs from hematopoietic stem cells (HSCs). Accordingly, CD123, TIM3 and CD96 will be used as markers to identify LSCs, as well as to perform MCL-1 dependency analyses.

First, bone marrow mononuclear cells (BMMC) from various stages of AML (e.g., newly diagnosed, complete remission, relapsed, refractory, on treatment) will be identified and acquired. Second, the acquired LSCs will be stained using CD34, CD38 and ALDH as LSC markers. Third, the acquired LSCs will be stained using CD34, CD38, CD123, TIM3 and CD96 as LSC markers, and the results will be compared with the results from CD34, CD38 and ALDH staining. Fourth, MCL-1 dependency assay will be performed on a population of LSCs based on cell-surface markers. Fifth, the LSCs will be sorted, and RNA isolated. Fifth, RNA_{seq} will be performed on RNA extracted from leukemic stem cells, and any remaining RNA will be stored for use in a qPCR assay to test MCL-1 dependency.

### Example 2. MCL-1 Dependency in Multiple Myeloma

CD38, CD45 and CD138 will be used as markers to identify MM stem cells, as well as to perform MCL-1 dependency analyses on stained samples to identify which populations respond to TMS-1. If CD38, CD45 and CD138 do not provide a clear population that responds to MCL-1 inhibition, then additional markers for MM stem cell identification will be identified and interrogated. Other potential markers include CD19, CD56, CD27, CD81 and CD117.

First, BMMC from MM samples will be identified and acquired. Second, the acquired MM samples will be stained using CD38, CD45 and CD138 as positive markers, and CD3 and CD20 as negative markers. Third, a MCL-1 dependency assay will be performed on the stained cells, and any response to TMS-1 will be identified in all of the above populations, individually or in combination. If the initial markers do not result in identification of an MCL-1-dependent population, then the first through third steps will be repeated with a new set of markers.

### Example 3. Alvocidib Followed by 7+3 Induction in Newly Diagnosed AML Achieves High Rates of MRD-Negative CR: Results of a Phase 1 Dose Escalation Study

Introduction. Overall outcomes remain poor in patients with acute myeloid leukemia (AML), which affects approximately 20,000 patients. MCL-1 represents a major regulator of apoptosis in AML, and inhibition of MCL-1 has been shown to lead to cell death and anti-leukemia activity in preclinical AML models (Leuk Res Rep. 2013; 2(1): 12-14). MCL-1 is up-regulated in AML and contributes to survival of leukemic stem cells, the population of cells responsible for minimal residual disease (MRD). The highly MCL-1 dependent leukemic stem cells that remain in MRD are responsible for relapse (Clin Invest. 2010; 120(6):2109-2118; Science. 2005 Feb. 18;307(5712):1101-4).

The cyclin-dependent kinase, CDK9, forms a complex with cyclin T1 (PTEF-b) which is recruited by bromodomain-containing protein-4 (BRD4) and mediator to superenhancer DNA complexes to regulate the activity of RNA Polymerase II, thereby leading to the transcription of genes regulating cell survival and proliferation, such as c-MYC and MCL-1.

Alvocidib inhibits CDK9, leading to suppression of RNA Polymerase II-mediated genes, including MCL-1 (J Exp Clin Cancer Res. 2018; 37(36)). Leukemic cells dependent on MCL-1 for survival have been shown to have heightened sensitivity to alvocidib-containing regimens (J Clin Oncol. 2015; 33 (suppl; abstr 7062), (Blood. 2015; 126:3799). In particular, alvocidib followed by cytarabine leads to enhanced leukemia cytotoxicity and has demonstrated clinical activity in a timed sequential therapy regimen (i.e., FLAM) (Leuk Res Rep. 2013; 2(1): 12-14), (Leuk Res. 2015 December; 39(12):1312-8).

Alvocidib has been investigated in a timed-sequential therapy approach in combination with cytarabine and mitoxantrone (ACM) in 405 patients with both newly diagnosed (n=256) and relapsed/refractory (n=149) AML with encouraging findings. However, alvocidib has not been studied in the context of conventional induction therapy with 7+3. Therefore, a phase 1 dose escalation study of alvocidib followed by 7+3 in newly diagnosed AML patients with non-favorable-risk cytogenetics was designed to assess the safety, maximal tolerated dose (MTD), and clinical activity of this regimen.

Methods: Study Population. This was a Phase 1, open-label, multicenter, dose-escalation study of alvocidib followed by cytarabine/daunorubicin (7+3) in patients with AML (NCT03298984). Patients aged 18-65 years with newly diagnosed, previously untreated AML and pathological confirmation of bone marrow (BM) blasts 20% were eligible. Key inclusion criteria included: Eastern Cooperative Oncology Group (ECOG) performance status (PS)≤2, serum creatinine level <1.8 mg/dL, alanine aminotransferase (ALT) and aspartate aminotransferase (AST)<5 times upper limit of normal (ULN), total bilirubin <2.0 mg/dL, and left ventricular ejection fraction (EF) >45%. Patients were excluded if they had previous treatment for AML (with the exception of hydroxyurea), and were diagnosed with acute promyelocytic leukemia (APL-M3) or core-binding factor AML ((CBF-AML: t(8;21); inv(16); t(16;16)). All patients provided written informed consent. This study was conducted as per the Declaration of Helsinki after approval by ethics committee of each participating center.

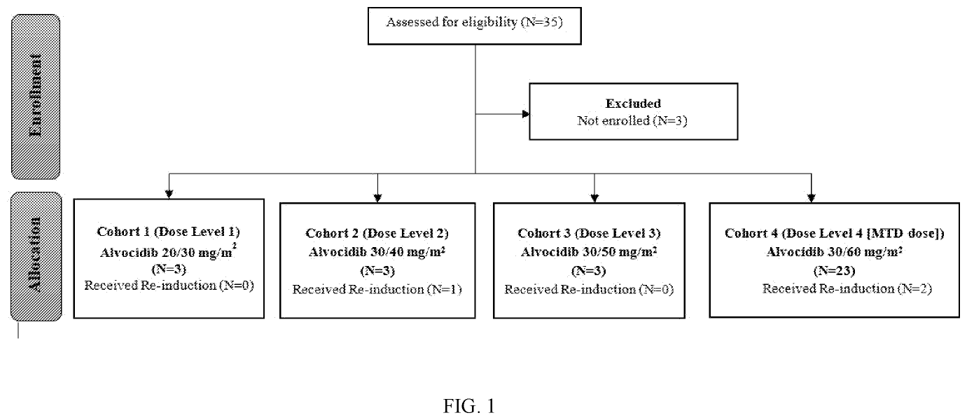

Treatment Plan: Induction. Alvocidib was dose-escalated starting at dose level 1: 20 mg/m² 30-minute IV bolus followed by 30 mg/m² IV infusion over 4 hours on Days 1-3 (FIG. 1). Daunorubicin 60 mg/m² IV bolus over 15 minutes was initiated on days 5-7, and cytarabine 100 mg/m²/day IV continuous 24-hour infusion was administered on Days 5-11. A bone marrow aspirate and biopsy was performed on day 14 (+/−3 days) and patients with residual leukemia (>5% bone marrow blasts and >10% cellularity) were recommended to receive a second induction cycle with alvocidib on days 1-3 (same dose level as induction) followed by daunorubicin 45 mg/m²/day IV over 15 minutes on days 5 and 6 and cytarabine 100 mg/m²/day IV continuous infusion over days 5-9 (“5+2”).

Consolidation. Patients who achieved a complete remission (CR) received 2 to 4 cycles of consolidation therapy with high dose cytarabine (HiDAC) 3 gm/m² IV every 12 hours on days 1, 3 and 5 upon full hematological recovery (absolute neutrophil count ≥1×10⁻³/L and platelets >1×10⁻¹/L). HiDAC was permitted to be dose reduced to 1.5 gm/m² IV every 12 hours on days 1, 3 and 5 in those >60 years. Allogeneic stem cell transplantation was permitted after induction per standard of care but was recommended after at least 1 cycle of HiDAC consolidation in those who achieved CR.

Response Assessments. Response assessment was performed by a bone marrow aspirate and biopsy at the time of full hematologic recovery or by day 50 and day 60 of 1 versus 2 induction cycles, respectively. Response to therapy was assessed by standardized ELN Guidelines. In those who achieved CR and received consolidation therapy, a bone marrow aspirate and biopsy was performed after cycle 2 of HiDAC, after completion of consolidation therapy, and at any suspicion of relapse.

Minimal Residual Disease (MRD) Analysis. MRD was assessed in bone marrow samples at the time of response by a uniform central assay (Hematologics, Inc.) in an exploratory cohort, as has been previously described. Samples were first incubated with antibodies for 20 minutes at room temperature. Red blood cells were then lysed at 37° C. using buffered NH₄Cl for 5 minutes, followed by centrifugation at 300 g. Cells were subsequently washed with PBS supplemented with 2% FCS and resuspended in 1% paraformaldehyde. Samples were analyzed on a FACSCalibur™ flow cytometer (Becton Dickinson Biosciences). 200,000 events were analyzed for each sample. Data analysis was performed with WinList™ software (Verity Software House). Two independent reviewers viewed each sample to arrive at a consensus on the data.

Mitochondrial Priming. Leukemia dependence on BH3 member proteins was assessed as previously described. Ryan J A, Brunelle J K, Letai A. Heightened mitochondrial priming is the basis for apoptotic hypersensitivity of CD4+CD8+ thymocytes. Proc Natl Acad Sci USA. 2010; 107(29):12895-12900. For evaluation of MCL-1 dependence, the MCL-1 binding protein, MS1, was used with modifications allowing for improved cell penetrance. The modified MCL-1 binding protein is referred to as T-MS1. Burrer C M, Foight G W, Keating A E, Chan G C. Selective peptide inhibitors of antiapoptotic cellular and viral Bcl-2 proteins lead to cytochrome c release during latent Kaposi's sarcoma-associated herpesvirus infection. Virus Res. 2016; 211:86-88. T-MS1 has higher potency and affinity for MCL-1 than NOXA. Foight G W, Ryan J A, Gulla S V, Letai A, Keating A E. Designed BH3 peptides with high affinity and specificity for targeting Mcl-1 in cells. ACS Chem Biol. 2014; 9(9): 1962-1968.

When added to cells, T-MS1 crosses the plasma membrane and antagonizes MCL-1, leading to mitochondrial outer membrane pore (MOMP) formation and depolarization of the mitochondria. Mitochondrial potential was assessed using the cationic dye, DiOC₂(3) (ThermoFisher, Waltham, Mass., USA). Fresh AML patient's BM cells were interrogated with T-MS1 treatment and the measurement of the resulting change in mitochondrial potential was compared to carbonyl cyanide m-chlorophenyl hydrazone (CCCP, Selleckchem, Houston, Tex., USA) and water treated controls. At least 2×10⁶ viable cells were assessed per patient. Priming was calculated as:

$$\% Priming = ( \frac{( Average \% Polarized Water - \% Polarized Peptide )}{Average \% Polarized Water} ) \times 100$$

For concordance with prior priming methodology and values, an additional calibration factor of 1.6 was applied to patient priming values.

Statistical Analysis. Alvocidib dose was escalated using a 3+3 design (FIG. 1). Successive cohorts of patients (3-6 per cohort) were treated with escalated doses until the maximal tolerated dose (MTD) was established. Intra-patient dose escalation was not permitted. Descriptive analysis was performed for the safety and efficacy endpoints. MTD for alvocidib was estimated by the highest alvocidib dose for which the incidence of DLT during Cycle 1 was less than 33% in combination with 7+3. Kaplan-Meier time-to-event analyses were performed on overall survival (OS), relapse-free survival and duration of remission. All statistical analyses were performed using SAS software version 9.4 or higher. Database lock was on Apr. 30, 2020.

Results: Patient Characteristics. Between December 2017 and September 2019, thirty-two patients were enrolled to this study. Patient characteristics are shown in Table 1. The median age was 58 years (range, 32-65 years), six (19%) had secondary AML (i.e., therapy-related AML or preexisting myelodysplasia, chronic myelomonocytic leukemia or myeloproliferative neoplasms), while 12 (32%) had AML with myelodysplasia-related changes (MRC) (defined by MDS-related cytogenetics or history of MDS/CMML). By ELN classification, 9 patients (28%) were favorable, 7 (22%) were intermediate, and 16 (50%) were adverse-risk. Similarly, 13 (41%) patients had unfavorable-risk cytogenetics by Southwest Oncology Group (SWOG) classification. The most common mutations seen in this cohort were NPM1 (31%), ASXL1 (19%) and RUNX1 (16%) mutations.

TABLE 1
Patient Characteristics

| | Alvocidib Dose | Alvocidib Dose | |
|---|---|---|---|
| | <MTD | at MTD | |
| | (<30/60 mg/m²) | (30/60 mg/m²) | Study Total |
| Patient Characteristics | N = 9 | N = 23 | N = 32 |
| Age, median (range), years | 60 (31, 65) | 51 (33, 65) | 58 (31, 65) |
| Age ≥60, years | 5 (55.6%) | 6 (26.1%) | 11 (34.4%) |
| Male, n (%) | 4 (44.4%) | 14 (60.9%) | 18 (56.3%) |
| Female, n (%) | 5 (55.6%) | 9 (39.1%) | 14 (43.8%) |
| ECOG performance status | | | |
| 0 | 5 (55.6%) | 8 (34.8%) | 13 (40.6%) |
| 1 | 3 (33.3%) | 13 (56.5%) | 16 (50.0%) |
| 2 | 1 (11.1%) | 2 (8.7%) | 3 (9.4%) |
| Bone Marrow Blasts (%) | 49 (23, 92) | 47 (12, 98) | 48 (12, 98) |
| median (range) | | | |
| Baseline WBC (×10⁹/L)-median | 3.1 (1.60, 15.54) | 3.99 (0.50, 16.00) | 3.87 (0.50, 16.00) |
| (range) | | | |
| Secondary AML, n (%) | 2 (22.2%) | 4 (17.4%) | 6 (18.8%) |
| t-AML | 0 (0.0%) | 3 (13.0%) | 3 (9.4%) |
| Prior MDS, n (%) | 1 (11.1%) | 2 (8.7%) | 3 (9.4%) |
| Prior CMML | 0 (0.0%) | 1 (4.3%) | 1 (3.1%) |
| Prior MPN | 1 (11.1%) | 0 (0.0%) | 1 (3.1%) |
| AML with MRC, n (%) | 4 (44.4%) | 8 (34.8%) | 12 (38.0%) |
| ELN classification, n (%) | | | |
| Favorable | 3 (33.3%) | 6 (26.1%) | 9 (28.1%) |
| Intermediate | 3 (33.3%) | 4 (17.4%) | 7 (21.9%) |
| Adverse-risk | 3 (33.3%) | 13 (56.5%) | 16 (50.0%) |
| SWOG cytogenetics | | | |
| classification, | | | |
| n (%) | | | |
| Favorable | 0 (0.0%) | 0 (0.0%) | 0 (0.0%) |
| Intermediate | 4 (44.4%) | 15 (65.2%) | 19 (59.4%) |
| Unfavorable | 5 (55.6%) | 8 (34.8%) | 13 (41.0%) |
| Unknown | 0 (0.0%) | 0 (0.0%) | 0 (0.0%) |
| Genetic mutations, n (%) | | | |
| NPM1 | 3 (33.3%) | 7 (30.4%) | 10 (31.3%) |
| FLT3 | 1 (11.1%) | 3 (13.0%) | 4 (12.5%) |
| CEBPA | 0 (0.00%) | 2 (8.7%) | 2 (6.3%) |
| ASXL1 | 0 (0.00%) | 6 (26.1%) | 6 (18.8%) |
| RUNX1 | 0 (0.00%) | 5 (21.7%) | 5 (15.6%) |
| EZH2 | 0 (0.00%) | 2 (8.7%) | 2 (6.3%) |
| IDH1 | 0 (0.00%) | 2 (8.7%) | 2 (6.3%) |
| IDH2 | 0 (0.00%) | 4 (17.4%) | 4 (12.5%) |
| TET2 | 0 (0.00%) | 4 (17.4%) | 4 (12.5%) |
| TP53 | 1 (11.1%) | 1 (4.3%) | 2 (6.3%) |
| U2AF1 | 1 (11.1%) | 2 (8.7%) | 3 (9.4%) |

Efficacy. Among all enrolled patients, overall response rate (ORR: CR+partial remission (PR)) and CR rates were 75% and 69%, respectively (Table 2). All patients who achieved a CR achieved full hematologic recovery. Among response-evaluable patients, the ORR and CR rates were 77% and 71%, respectively (one patient died on day 26 of re-induction without a response assessment). The CR rate was 100% (3 of 3) for dose level 1, but all 3 patients in that cohort had ELN favorable-risk disease. The CR rate was not significantly different across the other dose levels. Twenty-nine (91%) patients had no evidence of residual leukemia on day 14 assessment, whereas 3 (9%) received re-induction with alvocidib and 5+2 chemotherapy for residual leukemia on day 14. None of the patients who received re-induction therapy achieved CR. Overall CR rates were 89% (8 of 9), 71% (5 of 7), and 56% (9 of 16) for favorable-, intermediate-, and adverse-risk patients by ELN classification, respectively. Fifty percent of patients with secondary AML and AML, with MRC achieved CR. Of the 22 patients who achieved CR, 11 (50%) were MRD-negative by standardized flow cytometry and/or institutional standard assessment including 4/9 (44%) with ELN adverse-risk disease. At the recommended phase 2 dose level, 15/23 (65%) achieved CR.

TABLE 2
Clinical Activity - Response Assessments

| | l |
|---|---|
| | Response Evaluable |
| | Population |
| Response Characteristics | (N = 31) * |
| CR, n (%) | 22 (71.0%) |
| CRi (%) | 0 |
| Overall CR/CRi, n (%) | 22 (71.0%) |
| PR, n (%) | 2 (6.5%) |
| Overall Response Rate (CR + CRi + PR) | 24 (77.4%) |
| Overall CR subgroups, n (%) | |
| At MTD, (n = 23) | 15 (65.2%) |
| Age <60 years, (n = 21) | 16 (76.2%) |
| Age ≥60 years, (n = 10) | 6 (60.0%) |
| Secondary AML, (n = 5) | 3 (60.0%) |
| AML with MRC, (n = 11) | 6 (54.5%) |
| ELN-risk, n (%) | |
| Favorable (n = 9) | 8 (88.9%) |
| Intermediate (n = 7) | 5 (71.4%) |
| Adverse (n = 15) | 9 (60.0%) |
| SWOG cytogenetics risk, n (%) | |
| Favorable (n = 0) | 0 (0.0%) |
| Intermediate (n = 19) | 15 (78.9%) |
| Unfavorable (n = 12) | 7 (58.3%) |
| Unknown (n = 0) | 0 (0.0%) |

* One patient died on Day 26, prior to first planned response assessment, and is not included in the Response Evaluable Population
CR = complete remission,
MRD positive or unknown,
CRi = complete remission with incomplete recovery,
ELN = European LeukemiaNet,
MRC = myelodysplasia-related changes,
MTD = maximum tolerated dose,
PR = partial remission,
SWOG = Southwestern Oncology Group

Of the 22 patients who achieved CR, 19 (86%) received consolidation therapy with intermediate- or high-dose cytarabine consolidation (median number of cycles: 2; range: 1-4). Two patients proceeded directly to alloSCT after induction therapy without consolidation while 1 patient received 2 cycles of infusional cytarabine and daunorubicin (5+2) consolidation. Eleven (34%) proceeded to alloSCT (ELN favorable-risk: n=3, intermediate-risk: n=3, adverse-risk: n=5), all of whom achieved CR with induction therapy. Of the 11 patients who achieved CR and did not receive an alloSCT, 5 (45%), 2 (18%), and 4 (36%) were ELN favorable-risk, intermediate-risk and adverse-risk, respectively.

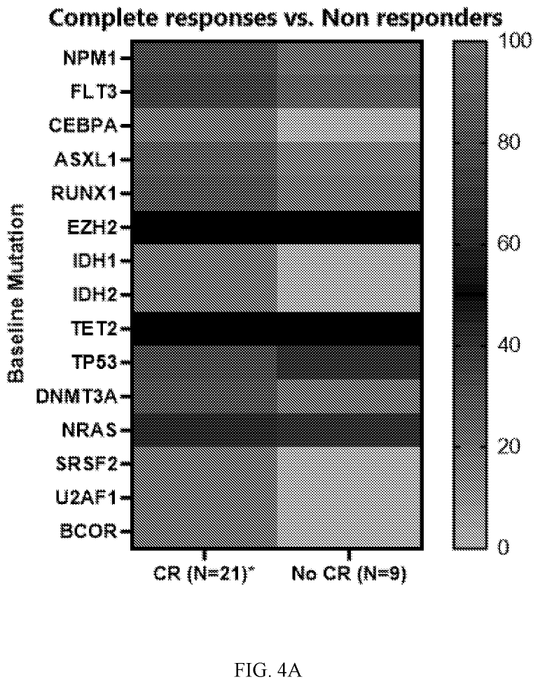

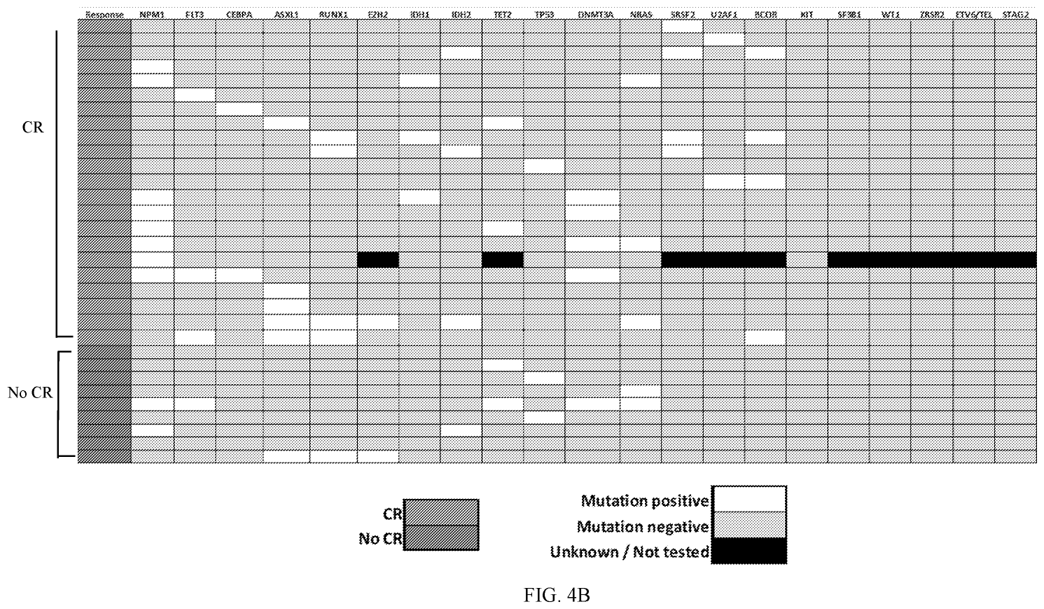

Genomic Signatures Predictive of Response. A heatmap of genomic signatures obtained at diagnosis by institutional standard NGS panel is shown in FIG. 4A. The majority of patients with NPM1 mutations achieved a CR (8/10=80% CR). One patient with NPM1 and IDH2 mutations achieved a PR (5-10% blasts by morphologic assessment despite no evidence of flow cytometric MRD), but eventually achieved CR after 1 cycle of HiDAC consolidation. Interestingly, overall CR rate was 83% (5/6) and 80% (4/5) among patients with ASXL1 and RUNX1 mutations, respectively. Only 1/3 (33%) patients with a TP53 mutation achieved CR. Notably, among patients with a previously classified genomic signature specific for secondary AML (i.e., ASXL1, BCOR, EZH2, SF3B1, SRSF2, STAG2, U2AF1, or ZRSR2), 11/12 (92%) achieved CR. Lindsley R C, Mar B G, Mazzola E, et al. Acute myeloid leukemia ontogeny is defined by distinct somatic mutations. Blood. 2015; 125(9):1367-1376.

Overall response among patients subdivided into the proposed genomic classification by Pappaemmanuil et al. was also analyzed (FIG. 4B). Papaemmanuil E, Gerstung M, Bullinger L, et al. Genomic Classification and Prognosis in Acute Myeloid Leukemia. New England Journal of Medicine. 2016; 374(23):2209-2221. The most common genomically defined subgroups in this cohort were AML with NPM1 mutation (n=10), and AML with mutated chromatin, RNA-splicing genes, or both (n=10). CR rates were 80% (8/10) and 90% (9/10) in patients classified as AML with NPM1 mutation and AML with mutated chromatin, RNA-splicing genes, or both, respectively.

MRD Exploratory Cohort. Twelve (38%) patients on the expansion cohort were included in a centralized MRD flow cytometry assessment. Nine (75%) achieved CR and 8/9 (89%) were determined to be MRD-negative (Table 3). One (11%) patient with adverse-risk disease achieved CR with evidence of MRD in this cohort.

TABLE 3
Central Analysis of Minimal Residual Disease

| | Central MRD |
|---|---|
| | Analysis Cohort |
| CR Characteristics | (N = 9) |
| MRD-Negative CR | 8/9 (89%) |
| Age | |
| <60 years | 6/7 (85.7%) |
| ≥60 years | 2/2 (100.0) |
| Secondary AML | 0/0 (0.0%) |
| AML with MRC | 1/1 (100.0%) |
| ELN risk | |
| Favorable | 4/4 (100.0%) |
| Intermediate | 1/1 (100.0%) |
| Adverse | 3/4 (75.0%) |
| SWOG cytogenetics risk | |
| Favorable | 0/0 (0.0%) |
| Intermediate | 7/7 (100.0%) |
| Unfavorable | 1/2 (50.0%) |
| Unknown | 0/0 (0.0%) |

ELN = European LeukemiaNet,
MRC = myelodysplasia-related changes,
MRD = minimal residual disease,
SWOG = Southwestern Oncology Group

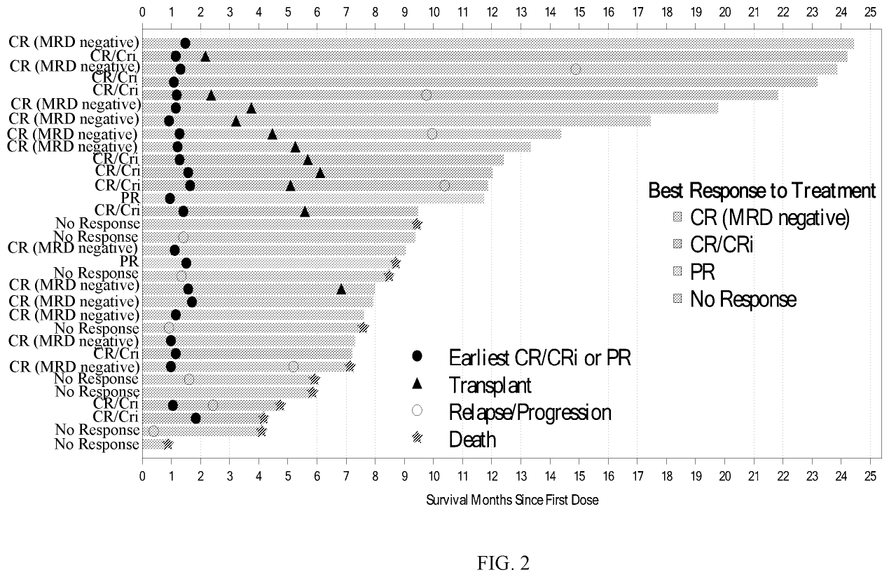

Clinical Outcomes. FIG. 2 displays a Swimmer's Plot of all patients enrolled (n=32). All of the patients (n=10) who have survived >1 year to date achieved CR while 6 had no evidence of MRD. Of the 22 CR patients, 7 (32%) relapsed to date (median duration of CR: 8.6 months; range: 1.4-13.6 months), four of whom received an alloSCT. Two (18%) patients who achieved CR without evidence of MRD relapsed (including one who underwent an alloSCT) while 9 (82%) who achieved CR without MRD remain in CR. Eleven (34%) patients died due to leukemia-related complications (n=9), septic shock during re-induction therapy (n=1), and disseminated mucormycosis after consolidation therapy while in CR (n=1).

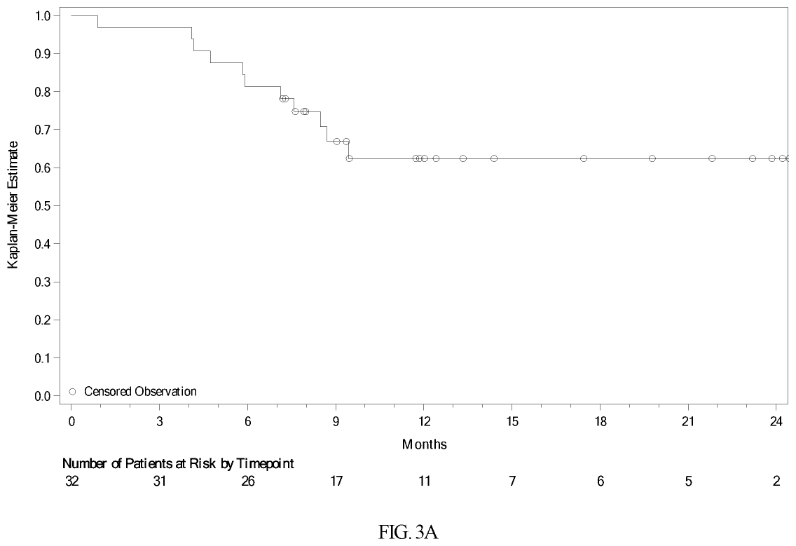

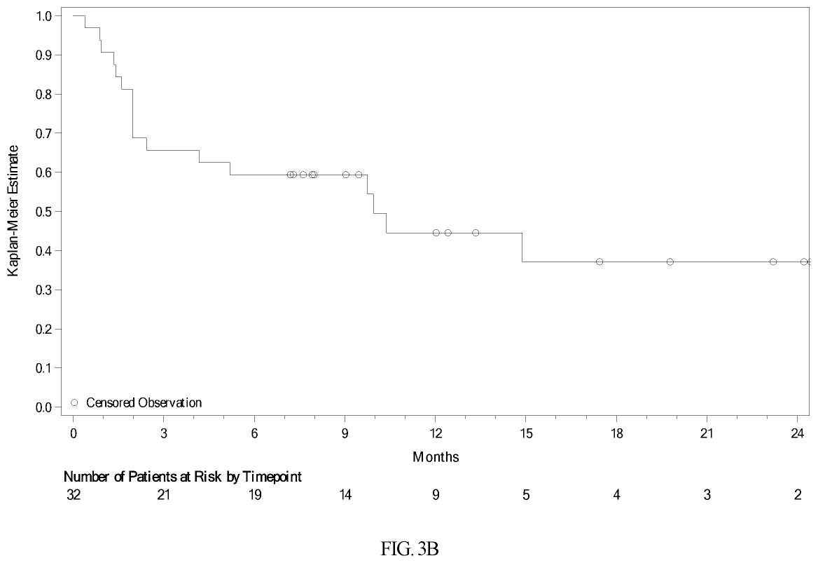

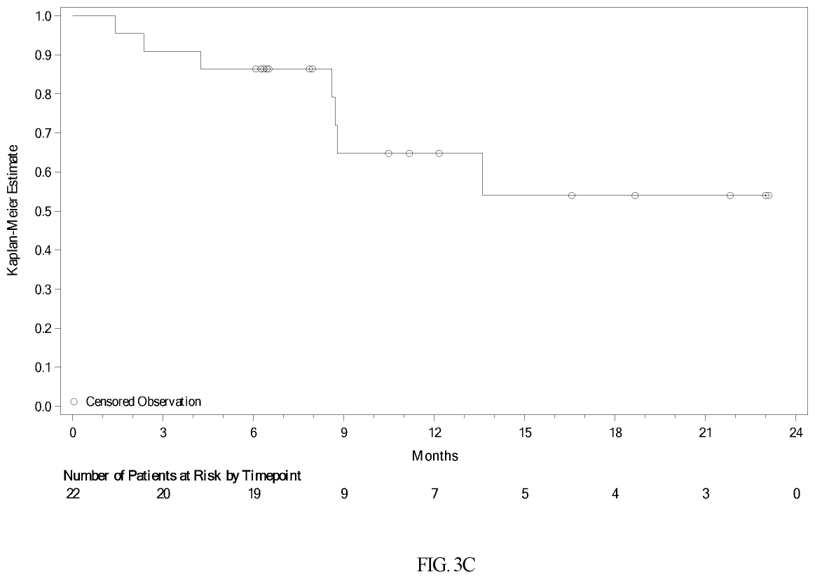

FIGS. 3A-3C depict the OS, EFS and RFS of the 32 patients enrolled on this study. Mean and median duration of follow-up was 11.4 and 9.2 months, respectively. The median OS was not reached due to relatively short duration of follow-up. Landmark 1-year OS was 62.4% (95% CI: 41.9, 77.4%). Median EFS was 10.0 months (95% CI: 2.0, NA) while median RFS was not reached. Landmark 1-year and 2-year EFS was 40.9% (95% CI: 21.9, 59.1%) and 34.1% (95% CI: 15.4, 53.8%), respectively. Landmark 1-year RFS was 59.5% (95% CI: 31.7, 79.1%).

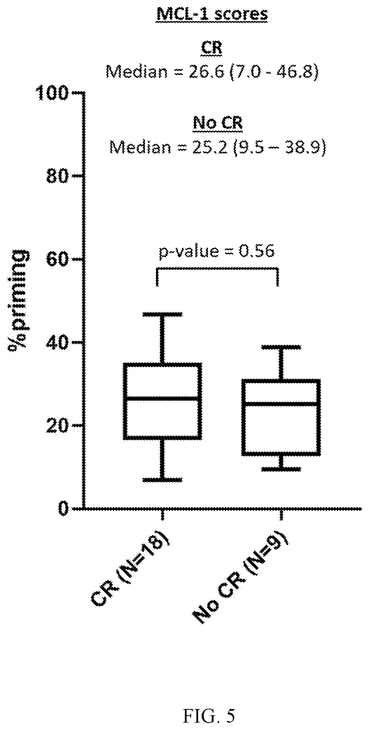

Mitochondrial Priming Correlates. MCL-1 dependence was assessed by mitochondrial profiling from pre-treatment diagnostic BM samples in 27/31 (87%) response-evaluable patients. In 3 patients, diagnostic BM correlates were not received, and 1 patient had insufficient quantity for analysis. Median MCL-1 score was 25.2% (range: 7.0-46.8%). There was no significant correlation of MCL-1 score among CR versus no CR (FIG. 5). Twelve (44%) and 2 (7%) patients had MCL-1 priming scores >30% and >40%, respectively. CR rate was 83% (10/12) and 67% (10/15) among those with MCL-1 scores greater than and less than 30%, respectively. Eleven out of 12 patients with genomically defined secondary AML had MCL-1 analysis performed (median MCL-1: 31.5%; Range: 7-38.9%). Of those analyzed, 7 (64%) patients had MCL-1 priming >30%, while 4 (36%) were <30%.

Discussion. The findings demonstrated that alvocidib combined with standard 7+3 induction chemotherapy is feasible and effective for younger patients (<65 years) with newly diagnosed AML.

The clinical activity of alvocidib followed by 7+3 seen in this study was encouraging and compares favorably to conventional chemotherapy regimens in this high-risk cohort. Achieving a CR is associated with longer relapse-free and OS when compared to non-responders and those achieving CR without full recovery (e.g., CRi). All of the patients who achieved CR on this study obtained full neutrophil and platelet recovery.

High CR rates were maintained across ELN genetic risk groups and were particularly notable in patients with secondary AML or AML with MRC. Patients with secondary AML and/or AML with MRC have dismal outcomes with conventional chemotherapy. In a randomized phase 3 trial of cytarabine plus amonafide versus 7+3 in newly diagnosed secondary AML, induction therapy with 7+3 yielded CR rates of 45% and median OS of 7 months. Further, a randomized phase 3 study of CPX-351, liposomal cytarabine and daunorubicin, versus 7+3 (daunorubicin 60 mg/m²) in newly diagnosed AML with MRC in patients 60-75 years revealed CR rates of 37% versus 26%, respectively, and approximately 33% of patients in both arms required re-induction therapy. In comparison, 50% of the patients with secondary AML or AML with MRC achieved CR on this study though 50% and 42% were <60 years, respectively.

Lindsley and colleagues previously reported that mutations in one of 8 genes (ASXL1, BCOR, EZH2, SF3B1, SRSF2, STAG2, U2AF1, or ZRSR2) is highly specific for secondary AML irrespective of a previously documented history of MDS or myeloid malignancies. Although ASXL1 mutations are defined as adverse-risk by ELN criteria, the other 7 mutations specific for secondary AML are not specifically listed as adverse-risk by standardized criteria. Recent data suggests that among older patients with intermediate-risk AML, mutations associated with secondary AML had significantly worse outcomes. A 2-class risk assessment from this analysis defined patients with adverse-risk and those with intermediate-risk with secondary AML mutations as “high-risk” disease.

Here, an encouraging CR rate of 92% (11/12) was noted in patients with a genomic profile consistent with secondary AML. Moreover, 64% of patients with this secondary AML genomic signature were found to be MCL-1 dependent (e.g., MCL-1 priming >30%), providing further rationale for alvocidib in this unique genomic subgroup with poor clinical outcomes.

MRD determined by multicolor flow cytometry, quantitative PCR, or next-generation sequencing is an important prognostic factor impacting the likelihood for relapse and survival after induction and prior to allogeneic stem cell transplant. San Miguel JsF, Vidriales MaB, Lopez-Berges C, et al. Early immunophenotypical evaluation of minimal residual disease in acute myeloid leukemia identifies different patient risk groups and may contribute to postinduction treatment stratification. Blood. 2001; 98(6):1746-1751; Kern W, Voskova D, Schoch C, Hiddemann W, Schnittger S, Haferlach T. Determination of relapse risk based on assessment of minimal residual disease during complete remission by multiparameter flow cytometry in unselected patients with acute myeloid leukemia. Blood. 2004; 104(10):3078-3085; Araki D, Wood B L, Othus M, et al. Allogeneic Hematopoietic Cell Transplantation for Acute Myeloid Leukemia: Time to Move Toward a Minimal Residual Disease-Based Definition of Complete Remission? Journal of Clinical Oncology. 2015; 34(4):329-336; Ivey A, Hills R K, Simpson M A, et al. Assessment of Minimal Residual Disease in Standard-Risk AML. New England Journal of Medicine. 2016; 374(5):422-433; Jongen-Lavrencic M, Grob T, Hanekamp D, et al. Molecular Minimal Residual Disease in Acute Myeloid Leukemia. New England Journal of Medicine. 2018; 378(13):1189-1199).

Multicolor flow cytometric evidence of MRD in CR1 leads to significantly worse outcomes compared with MRD-negative CR after induction therapy. In one study, patients were randomized to G-CSF priming or no priming during idarubicin and cytarabine induction. After one cycle of therapy, 164 patients in remission were assessed for MRD status and were considered MRD-negative if flow cytometric MRD was 0.1%. Of the 164 patients, 109 (66%) were MRD-negative. In the other study, 427 patients in remission after one or two cycles of daunorubicin/cytarabine or daunorubicin/clofarabine with or without gemtuzumab ozogamicin had samples analyzed for MRD. Of the 286 patients in CR after cycle 1, 145 (51%) had no detectable MRD with 0.1% as the sensitivity threshold. In both studies, MRD-positivity was associated with higher early relapse and lower relapse-free and overall survivals. Terwijn M, van Putten W L, Kelder A, et al. High prognostic impact of flow cytometric minimal residual disease detection in acute myeloid leukemia: data from the HOVON/SAKK AML 42A study. J Clin Oncol. 2013; 31(31):3889-3897; Freeman S D, Virgo P, Couzens S, et al. Prognostic relevance of treatment response measured by flow cytometric residual disease detection in older patients with acute myeloid leukemia. J Clin Oncol. 2013; 31(32):4123-4131.

In a large prospective MRD analysis from the National Cancer Research Institute (NCRI) AML17 trial, only 40% of younger patients treated with diverse “7+3” induction therapy backbones achieved CR without MRD after 1 cycle. Further, 5-year OS was 63% versus 44% among patients with CR and MRD-negative versus MRD-positive after 1 cycle of induction therapy. Freeman S, Hills R, Virgo P, et al. Measurable Residual Disease at Induction Redefines Partial Response in Acute Myeloid Leukemia and Stratifies Outcomes in Patients at Standard Risk Without NPM1 Mutations. Journal of clinical oncology: official journal of the American Society of Clinical Oncology. 2018;36(15).

In the study described herein, an exploratory cohort of 12 patients were treated at the recommended phase 2 dose to prospectively assess MRD status after one cycle of induction by centralized flow cytometry. Of these 12 patients, 9 (75%) achieved CR, and 8 (89% of CR patients, and 67% overall) achieved an MRD-negative CR, including 3 out of 4 (75%) ELN adverse-risk patients.

Mitochondrial profiling assesses the relative dependence of anti-apoptotic BCL-2 peptides in mediating cell survival within a tumor. NOXA is a BH3 sensitizer that selectively binds to and antagonizes MCL-1 leading to apoptosis in cells dependent on MCL-1 for survival. A NOXA mimetic peptide (T-MS1) “primes” cells for apoptosis, and high priming scores reflect cells that are considered to be MCL-1 dependent. An exploratory prospective analysis of MCL-1 dependence on response to alvocidib followed by 7+3 was conducted. The criteria of MCL-1 dependence was set at a priming threshold of >30%. There is no uniform criteria for defining MCL-1 dependence. No significant differences in response to alvocidib in patients with or without MCL-1 dependence was observed here, though this exploratory analysis was limited by small numbers of patients in each cohort. Nonetheless, 83% (10/12) of patients with MCL-1 dependence achieved CR. Given alvocidib's multi-CDK inhibitory activity and subsequent inhibition of RNA polymerase II, it is likely that alvocidib exerts anti-leukemia activity more broadly than direct MCL-1 inhibition. Thus, alvocidib may provide a therapeutic advantage over selective CDK9 and MCL-1 inhibitors even in patients considered to be MCL-1 dependent.

Hypomethylating agents in combination with venetoclax, a BCL-2 antagonist, has become the new standard-of-care for newly diagnosed older (>75 years) or unfit AML patients based on promising results from a phase 1 trial. Up-regulation of MCL-1 has been shown to be a dominant resistance mechanism to venetoclax-based strategies. Exploiting MCL-1 resistance and up-regulation during and after venetoclax treatment represents an attractive therapeutic approach in AML. Studies are ongoing exploring the role of alvocidib with or without low dose cytarabine in patients with relapsed/refractory AML after first-line venetoclax combination (NCT03969420) and the combination of alvocidib and venetoclax in relapsed/refractory AML (NCT03441555).

In conclusion, alvocidib administration prior to 7+3 induction is safe, feasible, and leads to encouraging clinical activity in newly diagnosed AML with non-favorable risk cytogenetics. Notably, alvocidib followed by 7+3 led to an impressive 92% CR rate in a genomically-defined signature of secondary AML that has traditionally poor clinical outcomes. In an exploratory cohort, high rates of MRD-negative CR rates were obtained with alvocidib followed by 7+3.

### Example 4. MCL-1 Dependency in Multiple Myeloma

CD38, CD45 and CD138 were used as markers to perform MCL-1 dependency analyses on stained samples to identify which populations respond to T-MS1 in accordance with Example 2.

The BH3 profiling assay was originally developed to detect MCL-1-dependent AML. In the BH3 assay, cells grown in culture or frozen archival samples are stained with antibody cocktail after recovery at 37° C. for 60 minutes in media containing RPMI+10% FBS+pen/strep. 250,000 cells per condition are treated for 30 minutes at 37° C. with T-MS1 in duplicate or vehicle (water) as a negative control. After T-MS1 treatment, cells are washed and stained with cationic dye, Dioc6, at 37° C. for 90 minutes. Mitochondrial depolarization is assessed via flow cytometric analysis. Priming percentage (%) is calculated using the following formula:

Priming %=(Control ˜Treatment)/Control*100.

A calibration factor of 1.6 is applied to T-MS1 priming results for consistency. T-MS1 is reconstituted in H₂O.

Alternatively, T-MS1-treated cells can be tested for MCL-1 dependency via cytochrome C release, which is an intracellular staining assay.

Several changes were made to the BH3 profiling assay to adapt it to MM samples. These changes involved changing out the antibody panel to identify plasma cells in patient BMMCs, in addition to acquiring the entire contents of each sample as opposed to only 25,000 events. The primary antibody panel for BH3 profiling of MM samples utilized the following markers: CD138-PE, CD38-ECD, CD45-PC7. Due to the differences in surface markers of AML blasts and malignant MM cells, a comprehensive analysis of the different stained markers on myeloma cells, individually and in combination, was performed. Aside from the necessary changes to the antibody profile and gating to differentiate MM cells, analysis followed that of the T-MS1-based MCL-1 dependency assay developed for AML. Upon implementing these changes, it was possible to process the various populations in MM samples and to profile their MCL-1 dependency.

The assay was performed using a total of 10 MM samples. Of these, 8 samples passed the quality control (QC) criterion of at least 40% polarized cells in the water-treated tube.

The priming percentages associated with the T-MS1 treated MM samples that passed the QC criterion are reported in Table 4. The results demonstrated an overall resistance to T-MS1 induced apoptosis. Only one patient sample (MM052) had uniform response in most cell populations. Of particular note, CD45^{dim} populations were highly sensitive to T-MS1 treatment-induced apoptosis (6/8 samples). However, due to the fact plasma cells in the bone marrow can be both CD45⁺ and CD45⁻, this marker alone is thought to be insufficient for identification of myeloma cells. When paired with other plasma cell markers, like CD38 and CD138, the response diminished significantly, and was almost completely eliminated when utilizing both other markers.

Nonetheless, two T-MS1-sensitive populations in MM were identified, corresponding to CD45^{dim} and CD45^{dim}CD138⁺. CD45^{dim} cells are traditionally considered progenitors, and are known to be apoptosis-resistant in other models. Without wishing to be bound by any particular theory, the major viewpoint is that clonotypic CD138⁻ cells represent MM stem cells (MMSC). For example, CD138⁻ cells are able to differentiate into CD138⁺ plasma cells, hinting that CD138⁻b cells contain properties of MMSC. Gao, M., et al. “Multiple Myeloma Cancer Stem Cells.” Oncotarget 7.23 (2016): 35466-77. However, clonotypic CD138⁺ plasma cells have stem properties (e.g., self-renewal, drug resistance, tumor-initiating potential). It may be that CD19⁻CD138⁺ plasma cells and CD19⁻CD138⁻ cells (pre-plasma cells) represent reversible, bidirectional phenotypic and functional states that share MMSC activity. Gao et al., 2016.

TABLE 4
Priming percentages of TMS-1-treated MM samples.

| | Dioc6 - Mitochondrial Membrane | | | | | | Cytochrome C | | |
|---|---|---|---|---|---|---|---|---|---|
| | Polarization | | | | | | Release | | |
| Population Priming | MM | MM | MM | MM | MM | MM | MM | MM | MM |
| % | 039 | 052 | 059 | 074 | 115 | 066 | 066 | 034 | 016 |
| Live | 37.8 | 78.9 | 46.3 | 33.3 | 48.5 | 27.5 | 36.1 | 53.3 | 0.0 |
| CD45+ | 28.3 | 83.2 | 28.9 | 18.4 | 34.8 | 19.6 | 26.8 | 29.4 | 0.0 |
| DC45 dim | 14.3 | 92.4 | 90.4 | 70.3 | 90.1 | 51.5 | 91.2 | 56.2 | 0.0 |
| CD38+ | 28.7 | 70.4 | 0.0 | 0.5 | 15.6 | 15.9 | 16.1 | 8.6 | 22.8 |
| CD45 dim CD38+ | 25.7 | 61.3 | 11.8 | 9.1 | 21.3 | 29.1 | 63.5 | 15.2 | 18.7 |
| CD138+ | 44.7 | 96.4 | 6.0 | 4.2 | 27.6 | 29.2 | 28.6 | 4.4 | 30.0 |
| CD45 dim CD138+ | 93.3 | 93.7 | 48.0 | 28.4 | 34.3 | 42.7 | 100.0 | | 27.1 |
| CD138+ | 8.8 | 43.1 | 14.0 | 9.9 | 8.9 | 15.5 | 1.8 | 10.1 | 9.9 |
| CD38+ | | | | | | | | | |
| CD45 dim CD38+ | 24.7 | 74.0 | 18.8 | 26.2 | 9.8 | 33.0 | 26.7 | | 19.2 |
| CD138+ | | | | | | | | | |

### Example 5. MCL-1 Dependency in MDS

CD13, CD33, CD34, CD45, CD3 and CD20 were used as markers to identify MDS blasts, and to perform MCL-1 dependency analyses. MDS blasts were positive for CD13, CD33 and CD34, dim for CD45 and negative for CD3 and CD20.

On day 1 of the MCL-1 dependency assay, 5×10⁴-1×10⁵ HS-5 feeder cells were plated into a 6-well plate, and allowed to attached overnight. HS-5 feeder cells improve viability of frozen MDS bone marrow mononuclear cells (BMMCs) after thawing. On day 2 of the assay, MDS BMMCs were thawed, and divided into three treatment groups corresponding to treatment with DMSO, treatment with 1 μM azacitidine or treatment with 2.5 μM azacitidine. On day 4, after a 48-hour treatment period, MCL-1 dependency of MDS blasts, lymphocytes and monocytes were separately assessed.

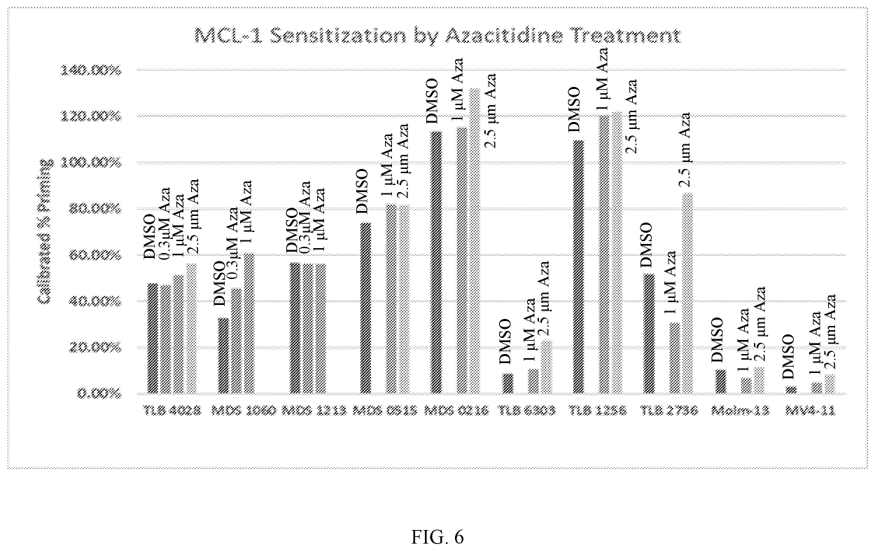

The results of the MCL-1 dependency assay of MDS blasts are shown in FIG. 6. Using an MCL-1 dependency cut-off of 40%, MCL-1 dependency was observed in 8/12 MDS bone marrow patient samples (DMSO control), and an increase in MCL-1 dependency for 10/12 patient samples was observed upon pre-treatment with azacitidine. Azacitidine pre-treatment in one patient sample, MDS1060, rendered MCL-1-independent blasts MCL-1 dependent.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of treating a hematologic cancer in a subject in need thereof, comprising administering to the subject an effective amount of alvocidib, or a prodrug thereof, or a pharmaceutically acceptable salt of the foregoing,
wherein the subject is in complete remission from the hematologic cancer and measurable residual disease (MRD)-positive following administration of a prior therapy that does not include alvocidib, or a prodrug thereof, or a pharmaceutically acceptable salt of the foregoing; and is MRD-negative following administration of alvocidib, or a prodrug thereof, or a pharmaceutically acceptable salt of the foregoing.

2. A method of treating a hematologic cancer in a subject in need thereof, comprising administering to the subject a maintenance therapy comprising an effective amount of alvocidib, or a prodrug thereof, or a pharmaceutically acceptable salt of the foregoing,
wherein the subject is in complete remission from the hematologic cancer following administration of an induction therapy for the hematologic cancer that does not include alvocidib, or a prodrug thereof, or a pharmaceutically acceptable salt of the foregoing; and is MRD-negative following administration of alvocidib, or a prodrug thereof, or a pharmaceutically acceptable salt of the foregoing.

3. A method of treating a hematologic cancer in a subject in need thereof, comprising administering to the subject a maintenance therapy comprising an effective amount of alvocidib, or a prodrug thereof, or a pharmaceutically acceptable salt of the foregoing, and one or more additional chemotherapeutic agents,
wherein the subject is in complete remission from the hematologic cancer following administration of an induction therapy for the hematologic cancer that does not include alvocidib, or a prodrug thereof, or a pharmaceutically acceptable salt of the foregoing.

4. The method of claim 3, wherein the subject is measurable residual disease (MRD)-negative following administration of alvocidib, or a prodrug thereof, or a pharmaceutically acceptable salt of the foregoing.

5. The method of any one of claims 1-4, wherein the hematologic cancer is multiple myeloma (MM), myelodysplastic syndrome (MDS), acute myeloid leukemia (AML), acute lymphoblastic leukemia (ALL), acute lymphocytic leukemia, chronic lymphogenous leukemia, chronic lymphocytic leukemia (CLL), chronic myeloid leukemia (CML), acute promyelocytic leukemia (APL), mantle cell lymphoma, diffuse large B-cell lymphoma (DLBCL), follicular lymphoma, or non-Hodgkin's lymphoma (NHL).

6. The method of claim 5, wherein the hematologic cancer is MM, AML, MDS, CLL or ALL.

7. The method of claim 6, wherein the hematologic cancer is MM.

8. The method of claim 6, wherein the hematologic cancer is AML.

9. The method of any one of claims 1-8, wherein the hematologic cancer is MCL-1 dependent.

10. The method of any one of claims 1-9, wherein the subject is elderly.

11. The method of any one of claims 1-9, wherein the subject is young.

12. The method of any one of claims 1-11, wherein the subject is unfit.

13. The method of any one of claims 1-11, wherein the subject is fit.

14. The method of any one of claims 1-13, wherein the subject is transplant-ineligible.

15. The method of any one of claims 1-13, wherein the subject is transplant-eligible.

16. The method of any one of claims 1-15, wherein alvocidib, or a prodrug thereof, or a pharmaceutically acceptable salt of the foregoing, is administered to the subject on a 28-day cycle.

17. The method of claim 16, wherein the prior therapy or induction therapy is administered on a cycle, and day 1 of the cycle of the prior therapy or induction therapy corresponds to day 1 of the 28-day cycle of alvocidib, or a prodrug thereof, or a pharmaceutically acceptable salt of the foregoing.

18. The method of any one of claims 1-17, wherein the prior therapy or induction therapy is administered on a cycle, and the subject was administered at least one cycle of the prior therapy or induction therapy prior to being administered alvocidib, or a prodrug thereof, or a pharmaceutically acceptable salt of the foregoing.

19. The method of any one of claims 1-18, wherein the subject continues to receive at least one of one or more therapeutic agents from the prior therapy or induction therapy for at least a portion of the time the subject is being administered alvocidib, or a prodrug thereof, or a pharmaceutically acceptable salt of the foregoing.

20. The method of any one of claims 1-19, wherein the subject continues to receive the prior therapy or induction therapy for at least a portion of the time the subject is being administered alvocidib, or a prodrug thereof, or a pharmaceutically acceptable salt of the foregoing.

21. The method of any one of claims 1-20, wherein administering alvocidib, or a prodrug thereof, or a pharmaceutically acceptable salt of the foregoing, to the subject comprises adding the alvocidib, or a prodrug thereof, or a pharmaceutically acceptable salt of the foregoing, to the prior therapy or induction therapy.

22. The method of any one of claims 1-20, wherein the subject is not receiving a therapeutic agent from the prior therapy or induction therapy while being administered alvocidib, or a prodrug thereof, or a pharmaceutically acceptable salt of the foregoing.

23. The method of any one of claims 1-22, further comprising detecting the measurable residual disease (MRD) status of the subject.

24. The method of claim 23, wherein the MRD status of the subject is detected prior to administering alvocidib, or a prodrug thereof, or a pharmaceutically acceptable salt of the foregoing, to the subject.

25. The method of claim 23, wherein the MRD status of the subject is detected after administering alvocidib, or a prodrug thereof, or a pharmaceutically acceptable salt of the foregoing, to the subject.

26. The method of any one of claims 23-25, wherein the MRD status of the subject is detected prior to and after administering alvocidib, or a prodrug thereof, or a pharmaceutically acceptable salt of the foregoing, to the subject.

27. The method of any one of claims 1-26, further comprising terminating administration of alvocidib, or a prodrug thereof, or a pharmaceutically acceptable salt of the foregoing, to the subject if the subject is determined to be measurable residual disease (MRD)-negative.

28. The method of any one of claims 1-27, wherein administration of alvocidib, or a prodrug thereof, or a pharmaceutically acceptable salt of the foregoing, is continued at least until the subject is measurable residual disease (MRD)-negative.

29. The method of any one of claims 2-28, further comprising terminating administration of the maintenance therapy to the subject if the subject is determined to be measurable residual disease (MRD)-negative.

30. The method of any one of claims 2-29, wherein administration of the maintenance therapy is continued at least until the subject is measurable residual disease (MRD)-negative.

31. A method of treating multiple myeloma in a subject in need thereof, comprising administering to the subject an effective amount of alvocidib, or a prodrug thereof, or a pharmaceutically acceptable salt of the foregoing, wherein:
the subject is in complete remission from the multiple myeloma and measurable residual disease (MRD)-positive following a prior maintenance therapy that includes lenalidomide, or a pharmaceutically acceptable salt thereof, and does not include alvocidib, or a prodrug thereof, or a pharmaceutically acceptable salt of the foregoing.

32. The method of claim 31, wherein the multiple myeloma is relapsed or refractory multiple myeloma.

33. The method of claim 31 or 32, wherein the subject is transplant-ineligible.

34. The method of any one of claims 31-33, wherein the subject continues to receive lenalidomide, or a pharmaceutically acceptable salt thereof, for at least a portion of the time the subject is being administered the compound of structural formula Ia, or a pharmaceutically acceptable salt thereof.

35. The method of any one of claims 31-34, wherein the subject continues to receive the prior maintenance therapy for at least a portion of the time the subject is being administered the compound of structural formula Ia, or a pharmaceutically acceptable salt thereof.

36. The method of any one of claims 31-35, wherein the prior maintenance therapy further comprises dexamethasone, or a pharmaceutically acceptable salt thereof.

37. The method of any one of claims 31-36, wherein the prior maintenance therapy includes from about 2.5 mg to about 25 mg of lenalidomide, or a pharmaceutically acceptable salt thereof, administered orally once daily on days 1-21 of a 28-day cycle.

38. The method of any one of claims 31-37, wherein the prior maintenance therapy includes about 25 mg of lenalidomide, or a pharmaceutically acceptable salt thereof, administered orally once daily on days 1-21 of a 28-day cycle.

39. The method of any one of claims 36-38, wherein the prior maintenance therapy includes about 40 mg of dexamethasone, or a pharmaceutically acceptable salt thereof, administered orally once daily on days 1-4, 9-12 and 17-20 of the 28-day cycle.

40. The method of any one of claims 36-38, wherein the prior maintenance therapy includes about 40 mg of dexamethasone, or a pharmaceutically acceptable salt thereof, administered orally once daily on days 1˜4 of the 28-day cycle.

41. The method of any one of claims 31-40, wherein the subject was administered the prior maintenance therapy for at least nine months prior to being administered alvocidib, or a prodrug thereof, or a pharmaceutically acceptable salt of the foregoing.

42. The method of any one of claims 31-41, further comprising administering to the subject lenalidomide, or a pharmaceutically acceptable salt thereof.

43. The method of any one of claims 31-42, further comprising administering to the subject the prior maintenance therapy.

44. A method of treating acute myeloid leukemia (AML) in a subject in need thereof, comprising administering to the subject an effective amount of alvocidib, or a prodrug thereof, or a pharmaceutically acceptable salt of the foregoing, wherein:
the subject is in complete remission from the AML and measurable residual disease (MRD)-positive following a prior induction therapy that includes cytarabine, or a pharmaceutically acceptable salt thereof, and daunorubicin, or a pharmaceutically acceptable salt thereof, and does not include alvocidib, or a prodrug thereof, or a pharmaceutically acceptable salt of the foregoing.

45. The method of claim 44, wherein the subject is unfit.

46. The method of claim 44 or 45, wherein the subject is elderly.

47. The method of any one of claims 44-46, wherein the cytarabine, or a pharmaceutically acceptable salt thereof, and daunorubicin, or a pharmaceutically acceptable salt thereof, are provided in a liposomal combination.

48. The method of any one of claims 44-47, wherein the prior induction therapy includes about 44 mg/m² daunorubicin and about 100 mg/m² cytarabine, administered via intravenous infusion over about 90 minutes on days 1, 3 and 5 of a cycle, or on days 1 and 3 of a cycle.

49. The method of any one of claims 44-48, wherein the alvocidib, or a prodrug thereof, or a pharmaceutically acceptable salt of the foregoing, is administered in the absence of an additional chemotherapeutic agent.

50. The method of any one of claims 44-49, further comprising administering to the subject a hypomethylating agent.

51. The method of claim 50, wherein the hypomethylating agent is azacitidine, or a prodrug thereof, or a pharmaceutically acceptable salt of the foregoing.

52. The method of claim 51, comprising administering to the subject from about 50 mg/m² to about 125 mg/m² azacitidine, or a pharmaceutically acceptable salt thereof, intravenously once daily.

53. The method of claim 52, comprising administering to the subject about 75 mg/m² azacitidine, or a pharmaceutically acceptable salt thereof, intravenously once daily.

54. The method of any one of claims 51-53, wherein the azacitidine, or a pharmaceutically acceptable salt thereof, is administered to the subject on days 1-7 of a 28-day cycle.

55. The method of any one of claims 51-53, wherein the azacitidine, or a pharmaceutically acceptable salt thereof, is administered to the subject on days 1-5, 8 and 9 of a 28-day cycle.

56. The method of claim 51, comprising administering to the subject from about 150 mg/m² to about 300 mg/m² azacitidine, or a pharmaceutically acceptable salt thereof, orally.

57. The method of claim 51 or 56, comprising administering to the subject CC-486 orally.

58. The method of claim 51, wherein the hypomethylating agent is a prodrug of azacitidine, or a pharmaceutically acceptable salt thereof.

59. The method of claim 58, wherein the prodrug of azacitidine is 2′,3′,5′-triacetyl-5-azacitidine, or a pharmaceutically acceptable salt thereof.

60. The method of claim 58, wherein the prodrug of azacitidine has the formula: 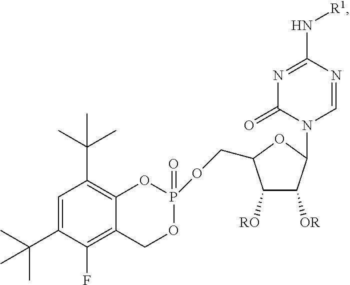
or a pharmaceutically acceptable salt thereof, wherein R and R¹ are each independently selected from H or CO₂(C₁-C₆)alkyl.

61. The method of claim 60, wherein each R is H and R¹ is CO₂(C₅-alkyl).

62. The method of claim 50, wherein the hypomethylating agent is decitabine, or a pharmaceutically acceptable salt thereof.

63. The method of claim 62, comprising administering to the subject from about 15 mg/m² to about 50 mg/m² decitabine, or a pharmaceutically acceptable salt thereof, intravenously or orally, once daily.

64. The method of claim 63, comprising administering to the subject about 20 mg/m² decitabine, or a pharmaceutically acceptable salt thereof, intravenously once daily for five days.

65. The method of claim 63, comprising administering to the subject about 35 mg/m² decitabine, or a pharmaceutically acceptable salt thereof, orally once daily for five days.

66. The method of claim 62, 63 or 65, comprising administering to the subject ASTX727 orally once daily for five days.

67. The method of any one of claims 31-66, wherein alvocidib, or a prodrug thereof, or a pharmaceutically acceptable salt of the foregoing, is administered to the subject on a 28-day cycle.

68. The method of claim 67, wherein the prior therapy or induction therapy is administered on a cycle, and day 1 of the cycle of the prior therapy or induction therapy corresponds to day 1 of the 28-day cycle of alvocidib, or a prodrug thereof, or a pharmaceutically acceptable salt of the foregoing.

69. The method of any one of claims 1-68, wherein the subject has one or more mutations in one or more of RUNX1, SRSF2, SF3B1, U2AF1, ZRSR2, ASXL1, EZH2, BCOR and STAG2.

70. A method of treating a cancer in a subject in need thereof, comprising administering to the subject a therapeutically effective amount of a therapy comprising alvocidib, or a prodrug thereof, or a pharmaceutically acceptable salt of the foregoing, wherein the subject has one or more mutations in one or more of RUNX1, SRSF2, SF3B1, U2AF1, ZRSR2, ASXL1, EZH2, BCOR and STAG2.

71. The method of claim 69 or 70, wherein the subject has one or more mutations in RUNX1.

72. The method of any one of claims 69-71, wherein the subject has one or more mutations in ASXL1.

73. The method of any one of claims 69-72, wherein the subject has one or more mutations in one, two, three, four or five of RUNX1, SRSF2, SF3B1, U2AF1, ZRSR2, ASXL1, EZH2, BCOR and STAG2.

74. The method of any one of claims 69-73, wherein the subject has one or more mutations in NPM1.

75. The method of any one of claims 70-74, wherein the cancer is a solid cancer.

76. The method of any one of claims 70-75, wherein the cancer is prostate cancer.

77. The method of claim 76, wherein the prostate cancer is castration-resistant prostate cancer.

78. The method of any one of claims 70-74, wherein the cancer is a hematologic cancer.

79. The method of claim 78, wherein the cancer is a leukemia.

80. The method of claim 79, wherein the leukemia is an acute leukemia.

81. The method of claim 80, wherein the acute leukemia is acute myeloid leukemia (AML).

82. The method of claim 81, wherein the AML is secondary AML.

83. The method of claim 81, wherein the AML is therapy-related AML.

84. The method of any one of claims 81-83, wherein the AML is relapsed or refractory.

85. The method of any one of claims 81-84, wherein the AML is resistant to venetoclax, or a pharmaceutically acceptable salt thereof, or venetoclax, or a pharmaceutically acceptable salt thereof, in combination with a hypomethylating agent.

86. The method of claim 79, wherein the hematologic cancer is a chronic leukemia.

87. The method of claim 78, wherein the cancer is a lymphoma.

88. The method of claim 78, wherein the cancer is multiple myeloma.

89. The method of claim 78, wherein the hematologic cancer is multiple myeloma, myelodysplastic syndrome (MDS), acute myeloid leukemia (AML), acute lymphoblastic leukemia (ALL), acute lymphocytic leukemia, lymphocytic lymphoma, mycosis fungoides, chronic lymphogenous leukemia, chronic lymphocytic leukemia (CLL), mantle cell lymphoma, diffuse large B-cell lymphoma, follicular lymphoma, or non-Hodgkin's lymphoma.

90. The method of claim 78, wherein the hematologic cancer is MDS.

91. The method of any one of claims 70-90, wherein the cancer is MCL-1 dependent.

92. The method of any one of claims 70-91, wherein the cancer is previously untreated.

93. The method of any one of claims 70-91, wherein the cancer is previously treated.

94. The method of claim 93, wherein the subject previously received venetoclax, or a pharmaceutically acceptable salt thereof.

95. The method of any one of claims 70-94, wherein the subject is elderly.

96. The method of any one of claims 1-95, wherein alvocidib, or a pharmaceutically acceptable salt thereof, is administered to the subject.

97. The method of claim 96, wherein from about 10 mg/m² to about 100 mg/m² alvocidib, or a pharmaceutically acceptable salt thereof, is administered to the subject per day.

98. The method of claim 97, wherein about 50 mg/m² alvocidib, or a pharmaceutically acceptable salt thereof, is administered to the subject per day.

99. The method of claim 97, wherein about 90 mg/m² alvocidib, or a pharmaceutically acceptable salt thereof, is administered to the subject per day.

100. The method of claim 97, wherein about 30 mg/m² alvocidib, or a pharmaceutically acceptable salt thereof, is administered to the subject by intravenous bolus of about 30 minutes in duration, and about 60 mg/m² alvocidib, or a pharmaceutically acceptable salt thereof, is administered to the subject by intravenous infusion of about 4 hours in duration.

101. The method of any one of claims 96-100, wherein alvocidib, or a pharmaceutically acceptable salt thereof, is administered to the subject once weekly for three consecutive weeks.

102. The method of any one of claims 96-100, wherein alvocidib, or a pharmaceutically acceptable salt thereof, is administered to the subject once every other week.

103. The method of any one of claims 96-100, wherein alvocidib, or a pharmaceutically acceptable salt thereof, is administered to the subject once daily for three consecutive days.

104. The method of any one of claims 96-103, wherein alvocidib, or a pharmaceutically acceptable salt thereof, is administered to the subject intravenously.

105. The method of any one of claims 96-99 and 101-104, wherein alvocidib, or a pharmaceutically acceptable salt thereof, is administered to the subject by intravenous bolus of from about 30 minutes to about 60 minutes in duration.

106. The method of any one of claims 96-99 and 101-104, wherein alvocidib, or a pharmaceutically acceptable salt thereof, is administered to the subject by intravenous infusion of about 60 minutes in duration.

107. The method of any one of claims 96-104, wherein alvocidib, or a pharmaceutically acceptable salt thereof, is administered to the subject by intravenous bolus of about 30 minutes in duration, followed by intravenous infusion of about 4 hours in duration.

108. The method of any one of claims 1-95, wherein a prodrug of alvocidib, or a pharmaceutically acceptable salt thereof, is administered to the subject.

109. The method of claim 108, wherein the prodrug of alvocidib is represented by the following structural formula: 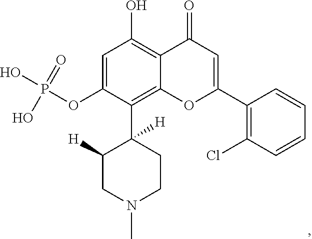
or a pharmaceutically acceptable salt thereof.

110. The method of claim 108 or 109, wherein the prodrug of alvocidib, or a pharmaceutically acceptable salt thereof, is administered to the subject orally.

111. The method of any one of claims 108-110, comprising administering from about 10 mg to about 50 mg per day of the prodrug of alvocidib, or a pharmaceutically acceptable salt thereof.

112. The method of claim 111, wherein about 8 mg of the prodrug of alvocidib, or a pharmaceutically acceptable salt thereof, is administered to the subject twice per day.

113. The method of claim 111, wherein about 16 mg of the prodrug of alvocidib, or a pharmaceutically acceptable salt thereof, is administered to the subject once per day.

114. The method of claim 111, wherein about 11 mg of the prodrug of alvocidib, or a pharmaceutically acceptable salt thereof, is administered to the subject twice per day.

115. The method of claim 111, wherein about 22 mg of the prodrug of alvocidib, or a pharmaceutically acceptable salt thereof, or a pharmaceutically acceptable salt thereof, is administered to the subject once per day.

116. The method of any one of claims 108-115, wherein the prodrug of alvocidib, or a pharmaceutically acceptable salt thereof, is administered on the first 14 days of a 21-day treatment cycle, and is not administered on days 15 to 21 of the 21-day treatment cycle.

117. The method of any one of claims 108-115, wherein the prodrug of alvocidib, or a pharmaceutically acceptable salt thereof, is administered on the first 21 days of a 28-day treatment cycle, and is not administered on days 22 to 28 of the 28-day treatment cycle.

118. The method of any one of claims 70-117, wherein the alvocidib, or a prodrug thereof, or a pharmaceutically acceptable salt of the foregoing, is administered in the absence of an additional chemotherapeutic agent.

119. The method of any one of claims 70-117, further comprising administering to the subject one or more additional chemotherapeutic agents.

120. The method of any one of claim 119, further comprising administering to the subject cytarabine, or a pharmaceutically acceptable salt thereof.

121. The method of claim 120, wherein the alvocidib, or a prodrug thereof, or a pharmaceutically acceptable salt of the foregoing, and the cytarabine, or a pharmaceutically acceptable salt thereof, are administered in the absence of an additional chemotherapeutic agent.

122. The method of claim 120 or 121, wherein the alvocidib, or a prodrug thereof, or a pharmaceutically acceptable salt of the foregoing, is administered on days 1 and 15 of a 28-day treatment cycle, and cytarabine, or a pharmaceutically acceptable salt thereof, is administered for ten consecutive days on days 3, 4, 5, 6, 7, 8, 9, 10, 11 and 12 of the 28-day treatment cycle.

123. The method of claim 122, wherein from about 15 mg/m² to about 40 mg/m² alvocidib, or a pharmaceutically acceptable salt thereof, is administered by intravenous bolus on day 1 of a 28-day treatment cycle; from about 40 mg/m² to about 80 mg/m² alvocidib, or a pharmaceutically acceptable salt thereof, is administered by intravenous bolus on day 15 of the 28-day treatment cycle; and from about 10 mg/m² to about 100 mg/m² cytarabine, or a pharmaceutically acceptable salt thereof, is administered per day by injection on days 3, 4, 5, 6, 7, 8, 9, 10, 11 and 12 of the 28-day treatment cycle.

124. The method of claim 120, further comprising administering to the subject daunorubicin or idarubicin, or a pharmaceutically acceptable salt of either of the foregoing.

125. The method of claim 124, wherein:
alvocidib, or a pharmaceutically acceptable salt thereof, or a prodrug of the foregoing, is administered to the subject on the first, second and third days of a treatment;
daunorubicin or idarubicin, or a pharmaceutically acceptable salt of the foregoing, is administered to the subject on the fifth, sixth and seventh days of the treatment; and
cytarabine, or a pharmaceutically acceptable salt thereof, is administered to the subject on the fifth, sixth, seventh, eighth, ninth, tenth, and eleventh days of the treatment.

126. The method of claim 125, wherein:
from about 5 mg/m² to about 50 mg/m² alvocidib, or a pharmaceutically acceptable salt thereof, per day, is administered by an intravenous bolus of from about 10 minutes to about 60 minutes in duration, and from about 10 mg/m² to about 65 mg/m² alvocidib, or a pharmaceutically acceptable salt thereof, per day, is administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;
from about 45 mg/m² to about 110 mg/m² daunorubicin, or a pharmaceutically acceptable salt thereof, per day, is administered by intravenous bolus of from about 5 minutes to about 30 minutes in duration on the fifth, sixth and seventh days of the treatment; and
from about 90 mg/m² to about 110 mg/m² cytarabine, or a pharmaceutically acceptable salt thereof, per day, is administered by intravenous infusion of from about 20 hours to about 28 hours in duration on the fifth, sixth, seventh, eighth, ninth, tenth, and eleventh days of the treatment.

127. The method of claim 126, wherein:
from about 5 mg/m² to about 50 mg/m² alvocidib, or a pharmaceutically acceptable salt thereof, per day, is administered by an intravenous bolus of from about 10 minutes to about 60 minutes in duration, and from about 10 mg/m² to about 65 mg/m² alvocidib, or a pharmaceutically acceptable salt thereof, per day, is administered by intravenous infusion of about 4 hours in duration, wherein the intravenous bolus and the intravenous infusion of alvocidib, or a pharmaceutically acceptable salt thereof, are administered to the subject on the first, second and third days of the treatment, and the intravenous infusion is initiated about 30 minutes after completion of the intravenous bolus;
about 60 mg/m² daunorubicin, or a pharmaceutically acceptable salt thereof, per day, is administered by intravenous bolus of from about 5 minutes to about 30 minutes in duration on the fifth, sixth and seventh days of the treatment; and
about 100 mg/m² cytarabine, or a pharmaceutically acceptable salt thereof, per day, is administered by intravenous infusion of from about 20 hours to about 28 hours in duration on the fifth, sixth, seventh, eighth, ninth, tenth, and eleventh days of the treatment.

128. The method of claim 119, further comprising administering to the subject a hypomethylating agent.

129. The method of claim 128, wherein the hypomethylating agent is azacitidine, or a pharmaceutically acceptable salt thereof.

130. The method of claim 129, wherein the azacitidine, or a pharmaceutically acceptable salt thereof, is administered to the subject once per day for from five to ten days.

131. The method of claim 130, wherein the azacitidine, or a pharmaceutically acceptable salt thereof, is administered to the subject once per day on days 1, 2, 3, 4, 5, 6 and 7 of a treatment schedule; and alvocidib, or a pharmaceutically acceptable salt thereof, is administered to the subject once on day 10 of the treatment schedule.

132. The method of claim 130, wherein the azacitidine, or a pharmaceutically acceptable salt thereof, is administered to the subject once per day on days 1, 2, 3, 4, 5, 8 and 9 of a treatment schedule; and alvocidib, or a pharmaceutically acceptable salt thereof, is administered to the subject once on day 10 of the treatment schedule.

133. The method of any one of claims 129-132, wherein from about 50 mg/m² to about 125 mg/m² azacitidine, or a pharmaceutically acceptable salt thereof, is administered to the subject per day.

134. The method of claim 128, wherein the hypomethylating agent is decitabine, or a pharmaceutically acceptable salt thereof.

135. The method of claim 134, wherein the decitabine, or a pharmaceutically acceptable salt thereof, is administered to the subject once per day for from three to ten consecutive days.

136. The method of claim 134 or 135, wherein from about 15 mg/m² to about 50 mg/m² decitabine, or a pharmaceutically acceptable salt thereof, is administered to the subject once per day.

137. The method of any one of claims 134-136, wherein from about 15 mg/m² to about 50 mg/m² decitabine, or a pharmaceutically acceptable salt thereof, is administered to the subject once per day on days 1, 2, 3, 4 and 5 of a treatment schedule; and alvocidib, or a pharmaceutically acceptable salt thereof, is administered to the subject once on day 8 of the treatment schedule.

138. The method of claim 120, further comprising administering to the subject mitoxantrone, or a pharmaceutically acceptable salt thereof.

139. The method of claim 138, wherein alvocidib, or a pharmaceutically acceptable salt thereof, is administered once daily on days 1-3 of a treatment schedule; cytarabine, or a pharmaceutically acceptable salt thereof, is administered on days 6-8 of the treatment schedule; and mitoxantrone, or a pharmaceutically acceptable salt thereof, is administered on day 9 of the treatment schedule.

140. The method of claim 138 or 139, wherein about 667 mg/m² cytarabine, or a pharmaceutically acceptable salt thereof, is administered per day; and about 40 mg/m² mitoxantrone, or a pharmaceutically acceptable salt thereof, is administered per day.
